(19) United States
(12) Patent Application Publication
(10) Pub. No.: US 2024/0156986 A1
(43) Pub. Date: May 16, 2024

(54) Gene therapy DNA vector based on gene therapy DNA vector VTvaf17 carrying the therapeutic gene selected from the group of COL1A1, COL1A2, P4HA1, P4HA2, COL7A1, CLCA2, ELN, and PLOD1 genes for increasing the expression level of these therapeutic genes, method of its production and use, Escherichia coli strain SCS110-AF/VTvaf17-COL1A1, or Escherichia coli strain SCS110-AF/VTvaf17-COL1A2, or Escherichia coli strain SCS110-AF/VTvaf17-P4HA1, or Escherichia coli strain SCS110-AF/VTvaf17-P4HA2, or Esch

(71) Applicants: CELL AND GENE THERAPY LTD, London (GB); OBSCHESTVO S OGRANICHENNOI OTVETSTVENNOSTJU "PRORYVNYE INNOVATSIONNYE TEKHNOLOGII", Moscow (RU)

(72) Inventor: Natalia SAVELIEVA, Wienna (AT)

(21) Appl. No.: 18/269,793

(22) PCT Filed: Dec. 20, 2019
(86) PCT No.: PCT/RU2019/000991

§ 371 Date: Jun. 27, 2023

(30) Foreign Application Priority Data
Dec. 27, 2018 (RU) 2018147082

(51) Int. Cl. A61K 48/00; A61K 38/39; A61K 38/44; C12N 15/85

(52) U.S. Cl. CPC A61K 48/005; A61K 38/39; A61K 38/44; C12N 15/85; C12Y 114/11002

(57) ABSTRACT

A gene therapy DNA vector based on the VTvaf17 gene therapy DNA vector carrying a target gene selected from the group of genes COL1A1, COL1A2, P4HA1, P4HA2, COL7A1, CLCA2, ELN, PLOD1 to increase the expression level of this target gene in humans and animals. Moreover, the gene therapy DNA vector VTvaf17-COL1A1 or VTvaf17-COL1A2 or VTvaf17-P4HA1 or VTvaf17-P4HA2 or VTvaf17-COL7A1 or VTvaf17-CLCA2 or VTvaf17-ELN or VTvaf17-PLOD1 has the nucleotide sequence SEQ ID NO. SEQ ID No. 3 or SEQ ID No. 4 or SEQ ID No. 5 or SEQ ID No. 6 or SEQ ID No. 7 or SEQ ID No. 8, respectively. The DNA vector contains no nucleotide sequences of viral origin and no antibiotic resistance genes, providing the possibility of its safe use for genetic therapy in humans and animals.

Gene therapy DNA vector based on gene therapy DNA vector VTvaf17 carrying the therapeutic gene selected from the group of COL1A1, COL1A2, P4HA1, P4HA2, COL7A1, CLCA2, ELN, and PLOD1 genes for increasing the expression level of these therapeutic genes, method of its production and use, Escherichia coli strain SCS110-AF/VTvaf17-COL1A1, or Escherichia coli strain SCS110-AF/VTvaf17-COL1A2, or Escherichia coli strain SCS110-AF/VTvaf17-P4HA1, or Escherichia coli strain SCS110-AF/VTvaf17-P4HA2, or Escherichia coli strain SCS110-AF/VTvaf17-COL7A1, or Escherichia coli strain SCS110-AF/VTvaf17-CLCA2, or Escherichia coli SCS110-AF/VTvaf17-ELN, or Escherichia coli strain SCS110-AF/VTvaf17-PLOD1 carrying the gene therapy DNA vector, method of production thereof, method of gene therapy DNA vector production on an industrial scale.

### FIELD OF THE INVENTION

The invention refers to genetic engineering and can be used in biotechnology, medicine, and agriculture for the manufacture of gene therapy products.

### BACKGROUND OF THE INVENTION

Gene therapy is an innovative approach in medicine aimed at treating inherited and acquired diseases by means of delivery of new genetic material into a patient's cells to compensate for or suppress the function of a mutant gene and/or treat a genetic disorder. The final product of gene expression may be an RNA molecule or a protein molecule. However, most physiological processes in the body are associated with the functional activity of protein molecules, while RNA molecules are either an intermediate product in the synthesis of proteins or perform regulatory functions. Thus, the objective of gene therapy in most cases is to inject the organism with genes that provide transcription and further translation of protein molecules encoded by these genes. Within the description of the invention, gene expression refers to the production of a protein molecule with amino acid sequence encoded by this gene.

COL1A1, COL1A2, P4HA1, P4HA2, COL7A1, CLCA2, ELN, and PLOD1 genes included in the group of genes play a key role in several processes in human and animal organisms. These genes are involved in the formation of extracellular matrix of the skin and other organs, as well as in a number of other processes.

The major components of extracellular matrix of the dermis are elastic fibres, collagen, and proteoglycans (27). Elastic fibres consist of fibrillin-rich microfibrils, glycoproteins, elastins, and some other proteins (13). The components of extracellular matrix of the dermis are interconnected by hyaluronic acid, thus forming the dermal network (27). The long collagen fibres formed by types I and III collagen intertwine, forming an intradermal network anchored at the border of the dermis and epidermis with type VII collagen (12).

During skin aging which is mainly due to internal mechanisms, collagen and elastic fibres in the dermis form cell structure (22). During aging caused by external factors, such as, for instance, under ultraviolet radiation, type I, III and, most significantly, type VII collagens are being lost (3; 30). In addition, long collagenic fibrils, elastic fibres, glycoproteins and glycosaminoglycans lose their ability to form a functional network of the extracellular matrix of dermis, instead they form unstructured fragments in the dermis (22). These changes in ECM are exacerbated by elastase activity produced by neutrophils migrating into the dermis as a result of inflammation or exposure to ultraviolet radiation (14), as well as activation of matrix metalloproteinases.

In addition to skin aging that is in most cases a natural process, the extracellular matrix of the dermis may be involved in the pathogenesis of various diseases, and disorders may be directly or indirectly associated with the expression of different genes involved in its formation. Moreover, since most of extracellular matrix molecules are involved in various biological processes that are not limited to skin, pathological and adverse conditions for an organism caused by insufficient expression of a number of genes may manifest as a skin structure damage that, nevertheless, are not limited to this tissue. For example, the collagen family is involved in the structural organisation and metabolism of many tissues in the body, including cartilage, bones, tendons, skin, and white of the eye (sclera). It was found that individual mutations in genes encoding collagens (for example, type I, III, or V) or collagen modifying enzymes (for example, lysyl hydroxylase, collagenase) cause different forms of Ehlers-Danlos syndrome that affect connective tissues (systemic dysplasia of the connective tissue) supporting the skin, bones, blood vessels, and other organs. Symptoms and signs of this disease vary within a wide range. The prevailing symptoms include hyper mobility of joints, pathological scarring and impaired wound healing, angiasthenia, and velvety hyperextensible skin.

Type I collagen is the most common form of collagen in humans. Type I collagen consists of two pro-α1 (I) chains and one pro-α2 (I) chain. The COL1A1 gene encodes the pro-α1 (I) chain, the COL1A2 gene-pro-α2 (I).

A mutation in the COL1A1 gene that causes infantile cortical hyperostosis or Caffey disease is described. This condition features soft tissues oedema (for example, muscles), pain, and excessive formation of new bone tissue (hyperostosis). Bone abnormalities mainly affect the jawbone, clavicles, (collarbones) and diaphysis of long limb bones.

Another hereditary disease due to mutations in the collagen genes and causing diffuse abnormal brittleness of bones, sometimes accompanied by sensorineural hearing loss, blue scleros, imperfect dentinogenesis, and hypermobility of joints is a brittle bone disease. 90% of people with one of the main types of disease have mutations in COL1A1 or COL1A2 genes. The gene therapy approach is being discussed as one of the promising directions in therapy for this syndrome. A clinical case of experimental treatment was also described, in which bone marrow mesenchymal stem cells expressing normal collagen genes were injected into a patient with brittle bone disease, resulting in a noticeable therapeutic effect (18).

Type VII collagen is the main structural component in the skin included in the anchoring fibrils. These fibrils are located in the region that constitutes a bilayer membrane located between epidermis and dermis. Collagen fibrils hold two layers of skin together, connecting epidermal base membrane with dermis.

The COL7A1 gene encodes type VII collagen. Three pro-α1(VII) chains twist together to form a triplex procollagen molecule. Procollagen molecules are secreted by the cell and processed by enzymes to remove extra protein segments from the ends. Once these molecules are processed, they arrange themselves into long, thin bundles of mature type VII collagen.

Mutations in the COL7A1 gene cause dystrophic epidermolysis bullosa. Blisters most commonly occur in areas of minor injuries, such as the extensor surfaces of elbows and back of the hands and feet. Healing results in scarring, superficial epidermal cysts and hyperpigmentation. Some patients have nail dystrophy. Extracutaneous manifestations, including injuries to the urinary and gastrointestinal tracts, outer eye membranes, chronic anemia, osteoporosis, and growth retardation frequently occur. Patients with epidermolysis bullosa are at a high risk of cancer, in particular, formation of aggressive squamous cell carcinomas. According to DEBRA International, one patient per 50-100 thousand people is born in the world.

Gene therapy approaches to the treatment of epidermolysis bullosa include different experimental treatments. In several studies for the correction of COL7A1 gene mutations, ex vivo genome editing technologies (20), microinjections of linear DNA molecules encoding the COL7A1 gene (19), cDNA integration using integrase enzymes (23), intradermal injections of lentiviral vectors (34), mutation repair technology based on TALEN nucleases (24), as well as injection of autologous cells, i.e. modified fibroblasts or keratinocytes using various retroviral vectors (11, 6, 7) were successfully used. Currently, clinical trials of such approaches are in different phases of study (NCT01263379, NCT02810951).

An important step in the generation and stabilization of collagen molecules are posttranslational modifications, i.e. proline hydroxylation necessary for stabilization of the collagen triple helix, and lysine hydroxylation for the subsequent covalent bonding between collagen molecules during collagen fibril assembly. The enzymes that are involved in these modification processes are prolyl 4-hydroxylase and lysyl 5-hydroxylase, respectively.

Prolyl 4-hydroxylase consists of 2 alpha and 2 beta subunits. Alpha subunits refer to several types and are encoded by P4HA1 and P4HA2 genes. Mutations in the P4HA1 gene can cause one of the forms of Ehlers-Danlos syndrome described above. A mutation in the human P4HA1 gene is also described that causes a unique phenotype of pathology featuring early joint hypermobility, articular contractures, muscle weakness, and bone dysplasia, as well as myopia (35). It is reported that smoking causes suppression of P4HA1 gene expression. The authors of this study associate this phenomenon with the induction of collagen metabolic disorders in the vessel walls of smokers and, as a result, atherosclerosis frequency rise and aneurysms (25). Mutations in the P4HA2 gene cause myopia (21). Suppression of P4HA2 gene transcription also occurs in lymphoid cells, which may be associated with the pathogenesis of oncological disease (9). The change in P4HA1 expression is proposed as one of the methods for screening the effectiveness of anti-aging cosmetic preparations derived from plant materials (28).

A large quantity of elastin, i.e. protein encoded by ELN gene contains in the extracellular matrix of connective tissue together with collagen. Elastin performs important functions in organs subjected to constant elongation and compression, for example, in arteries, lungs, skin, tendons, and various sphincters (39). Elastin and collagen fibres help the organs to restore their original size after elongation, for example, in case of skin pinching or after bladder emptying (38). Cross links between the fibres are formed in insufficient quantities or not formed at all with reduction in normal elastin form formation. As a result, the tensile strength of elastic tissues decreases and such disorders as thinning, flaccidity, elongation are manifested, i.e. their elastic properties are lost. Such disorders are clinically presented as cardiovascular changes (aneurysms and aortic ruptures, heart valve defects), frequent pneumonia, and pulmonary emphysema (36). In case of disturbance of elastin synthesis in the body due to the mutation of ELN gene supra-aortic stenosis is developed (5). Furthermore, the genetic engineering approach using viral vectors expressing the ELN gene was successfully applied to modify cells for cell therapy aimed at healing of soft tissue injuries in laboratory animals (16).

The PLOD1 gene encodes the lysyl hydroxylase 1. This enzyme modifies lysine producing hydroxylysine. Hydroxylysine in collagen molecules is necessary for the formation of cross links between collagen fibres. Like most previous genes associated with the synthesis and formation of extracellular matrix, mutations in the PLOD1 gene are associated with the development of Ehlers-Danlos syndrome (33). There is also evidence that the polymorphism of this gene may be associated with bone density and risks of osteoporosis (31).

The CLCA2 gene encodes a regulator of Ca channels and is expressed in various epithelial tissues (skin, corneal epithelium, esophagus, larynx, and vaginal epithelium) (2). Experiments on rats showed that the expression of this gene in skin keratinocytes is considerably reduced under ultraviolet radiation (1). While studying the mechanisms of pathogenesis of atopic dermatitis, researchers found that the expression of this gene is necessary to protect caratinocyte cells from apoptosis under hyperosmotic shock (caused by an insufficient quantity of water) with adhesion deficiency in the epidermis when CLCA2 is suppressed. Thus, the CLCA2 gene is necessary for the adaptation and survival of epithelial cells under insufficient moisture conditions (29).

Thus, the background of the invention suggests that mutations in COL1A1, COL1A2, P4HA1, P4HA2, COL7A1, CLCA2, ELN, and PLOD1 genes or insufficient expression of proteins encoded by these genes are associated with the development of a spectrum of diseases, including, but not limited to, hereditary and acquired pathological conditions associated with disorders in the organisation of extracellular matrix of the skin and other organs, resulting in both pathological processes and adverse conditions that fall within the generally accepted standard limits, but can be improved, as well as processes not directly related to the extracellular matrix. This is why COL1A1, COL1A2, P4HA1, P4HA2, COL7A1, CLCA2, ELN, and PLOD1 genes are grouped within this patent. Genetic constructs that provide expression of proteins encoded by COL1A1, COL1A2, P4HA1, P4HA2, COL7A1, CLCA2, ELN, and PLOD1 genes can be used to develop drugs for the prevention and treatment of different diseases, as well as pathological and adverse conditions.

Moreover, these data suggest that insufficient expression of proteins encoded by COL1A1, COL1A2, P4HA1, P4HA2, COL7A1, CLCA2, ELN, and PLOD1 genes included in the group of genes is associated not only with pathological conditions, but also with a predisposition to their development. Also, these data indicate that insufficient expression of these proteins may not appear explicitly in the form of a pathology that can be unambiguously described within the framework of existing clinical practice standards (for example, using the ICD code), but at the same time cause conditions that are unfavourable for humans and animals and associated with deterioration in the quality of life.

Analysis of approaches to increase the expression of therapeutic genes implies the practicability of use of different gene therapy vectors.

Gene therapy vectors are divided into viral, cell and DNA vectors (8). Recently, gene therapy has paid increasingly more attention to the development of non-viral gene delivery systems with plasmid vectors topping the list. Plasmid vectors are free of limitations inherent in cell and viral vectors. In the target cell, they exist as an episome without being integrated into the genome, while producing them is quite cheap, and there is no immune response or side effects caused by the administration of plasmid vectors, which makes them a convenient tool for gene therapy and prevention of the genetic diseases (DNA vaccination) (15).

However, limitations of plasmid vectors use in gene therapy are: 1) presence of antibiotic resistance genes for the production of constructs in bacterial strains; 2) the presence of various regulatory elements represented by sequences of viral genomes; 3) length of therapeutic plasmid vector that determines the efficiency of vector delivery to the target cell.

It is known that the European Medicines Agency deems it necessary to refrain from adding antibiotic resistance marker genes to newly engineered plasmid vectors for gene therapy (26) (Reflection paper on design modifications of gene therapy medicinal products during development/14 Dec. 2011 EMA/CAT/GTWP/44236/2009 Committee for advanced therapies). This recommendation is primarily related to the potential danger of the DNA vector penetration or horizontal antibiotic resistance gene transfer into the cells of bacteria found in the body as part of normal or opportunistic microflora. Furthermore, the presence of antibiotic resistance genes significantly increases the length of DNA vector, which reduces the efficiency of its penetration into eukaryotic cells.

It is important to note that antibiotic resistance genes also make a fundamental contribution to the method of production of DNA vectors. If antibiotic resistance genes are present, strains for the production of DNA vectors are usually cultured in medium containing a selective antibiotic, which poses risk of antibiotic traces in insufficiently purified DNA vector preparations. Thus, production of DNA vectors for gene therapy without antibiotic resistance genes is associated with the production of strains with such distinctive feature as the ability for stable amplification of therapeutic DNA vectors in the antibiotic-free medium.

In addition, the European Medicines Agency recommends avoiding the presence of regulatory elements in therapeutic plasmid vectors to increase the expression of therapeutic genes (promoters, enhancers, post-translational regulatory elements) that constitute nucleotide sequences of genomes of various viruses (Draft Guideline on the quality, non-clinical and clinical aspects of gene therapy medicinal products, http://www.ema.europa.eu/docs/en_GB/document_library/Scientific_guideline/2015/05/WC500187020.pdf). Although these sequences can increase the expression level of the therapeutic transgene, however, they pose risk of recombination with the genetic material of wild-type viruses and integration into the eukaryotic genome. Moreover, the relevance of overexpression of the particular gene for therapy remains an unresolved issue.

The size of the therapy vector is also essential. It is known that modern plasmid vectors often have unnecessary, non-functional sites that increase their length substantially (17) (Mairhofer J, Grabherr R. //Mol Biotechnol. 2008.39(2): 97-104). For example, ampicillin resistance gene in pBR322 series vectors, as a rule, consists of at least 1000 bp, which is more than 20% of the length of the vector itself. A reverse relationship between the vector length and its ability to penetrate into eukaryotic cells is observed; DNA vectors with a small length effectively penetrate into human and animal cells. For example, in a series of experiments on transfection of HELA cells with 383-4548 bp DNA vectors it was shown that the difference in penetration efficiency can be up to two orders of magnitude (100 times different) (10).

Thus, when selecting a DNA vector, for reasons of safety and maximum effectiveness, preference should be given to those constructs that do not contain antibiotic resistance genes, the sequences of viral origin and length of which allows for the effective penetration into eukaryotic cells. A strain for production of such DNA vector in quantities sufficient for the purposes of gene therapy should ensure the possibility of stable DNA vector amplification using antibiotic-free nutrient media.

Example of usage of the recombinant DNA vectors for gene therapy is the method of producing a recombinant vector for genetic immunisation (U.S. Pat. No. 9,550,998 B2. The plasmid vector is a supercoiled plasmid DNA vector that is used for the expression of cloned genes in human and animal cells. The vector contains an origin of replication, regulatory elements comprising human cytomegalovirus promoter and enhancer, and regulatory sequences from the human T-cell lymphotropic virus.

The vector is accumulated in a dedicated E. coli strain free of antibiotics through antisense complementation of sacB gene inserted into the strain by means of bacteriophage. The disadvantage of this invention is the presence of regulatory elements in the composition of DNA vector that constitute sequences of viral genomes.

Patents and applications described below may be considered as prototypes of the present invention.

Application No. WO2001042285A2 describes a method for restoring extracellular matrix and preventing its degradation, including gene therapy approach and vectors expressing genes containing sequences selected from the group of sequences (SEQ1-SEQ21) that are expressed during formation and maintenance of the extracellular matrix. The disadvantage of this invention is the approach to the selection of sequences that in this invention is not based on the physiological function of proteins encoded by these genes, but on the transcription analysis of different sequences. Also this invention does not provide justification for the effectiveness and safety in use of a particular vector for gene therapy.

Application No. JPH0823979A describes a gene therapy approach to improve the formation of extracellular matrix, including by expressing collagen and/or prolyl hydroxylase enzymes that provide biochemical reactions during the formation of collagen fibres. The disadvantage of this invention is the limited way of modulating the formation of extracellular matrix only through the hydroxylation reaction of proline in collagen molecules, another disadvantage of this invention is the use of baculovirus vectors.

Application No. WO2002094876A2 describes ways to control the expression of mucin in the lung tissues using gene therapy constructs that provide CLCA2 expression. The disadvantage of this invention is the limited use and the vague safety requirements applied to the vectors.

Thus, at the present background of invention, there is a need for an invention of effective and safe gene therapy approach that allows to increase the expression of genes involved in the formation of the extracellular matrix, taking into account both direct structural molecules (such as collagens and elastin) and enzymes that provide their post-translational modifications.

### DISCLOSURE OF THE INVENTION

The purpose of this invention is to construct gene therapy DNA vectors in order to increase the expression level of genes selected from the group of genes: COL1A1, COL1A2, P4HA1, P4HA2, COL7A1, CLCA2, ELN, and PLOD1 in humans and animals, combining the following properties:

I) Efficiency of gene therapy DNA vector in order to increase the expression level of therapeutic genes in eukaryotic cells.

II) Possibility of safe use in gene therapy of human beings and animals due to the absence of regulatory elements representing the nucleotide sequences of viral genomes in the gene therapy DNA vector.

III) Possibility of safe use in the gene therapy of human beings and animals due to the absence of antibiotic resistance genes in the gene therapy DNA vector.

IV) Producibility and constructability of gene therapy DNA vector on an industrial scale.

Item II and III are provided for herein in line with the recommendations of the state regulators for gene therapy medicines and, specifically, the requirement of the European Medicines Agency to refrain from adding antibiotic resistance marker genes to newly engineered plasmid vectors for gene therapy (Reflection paper on design modifications of gene therapy medicinal products during development/14 Dec. 2011 EMA/CAT/GTWP/44236/2009 Committee for advanced therapies) and refrain from adding viral genomes to newly engineered plasmid vectors for gene therapy (Guideline on the quality, non-clinical and clinical aspects of gene therapy medicinal products/23 Mar. 2015, EMA/CAT/80183/2014, Committee for Advanced Therapies).

The purpose of the invention also includes the construction of strains carrying these gene therapy DNA vectors for the development and production of these gene therapy DNA vectors on an industrial scale.

The specified purpose is achieved by using the produced gene therapy DNA vector based on the gene therapy DNA vector VTvaf17 for treatment of diseases associated with disorders of formation of the extracellular matrix of the skin and other organs, skin structure, wound healing disorders, for prevention of skin ageing caused by internal and external factors, for treatment of hereditary and acquired connective tissue diseases, including Ehlers-Danlos syndrome, epidermolysis bullosa, Caffey disease, osteogenesis imperfecta, pathological scarring, formation of scars, superficial epidermal cysts, and hyperpigmentation while the gene therapy DNA vector VTvaf17-COL1A1 contains the coding region of COL1A1 therapeutic gene cloned to gene therapy DNA vector VTvaf17 with the nucleotide sequence SEQ ID No. 1, the gene therapy DNA vector VTvaf17-COL1A2 contains the coding region of COL1A2 therapeutic gene cloned to gene therapy DNA vector VTvaf17 with the nucleotide sequence SEQ ID No. 2, the gene therapy DNA vector VTvaf17-P4HA1 contains the coding region of P4HA1 therapeutic gene cloned to gene therapy DNA vector VTvaf17 with the nucleotide sequence SEQ ID No. 3, the gene therapy DNA vector VTvaf17-P4HA2 contains the coding region of P4HA2 therapeutic gene cloned to gene therapy DNA vector VTvaf17 with the nucleotide sequence SEQ ID No. 4, the gene therapy DNA vector VTvaf17-COL7A1 contains the coding region of COL7A1 therapeutic gene cloned to gene therapy DNA vector VTvaf17 with the nucleotide sequence SEQ ID No. 5, the gene therapy DNA vector VTvaf17-CLCA2 contains the coding region of CLCA2 therapeutic gene cloned to gene therapy DNA vector VTvaf17 with the nucleotide sequence SEQ ID No. 6, the gene therapy DNA vector VTvaf17-ELN contains the coding region of ELN therapeutic gene cloned to gene therapy DNA vector VTvaf17 with the nucleotide sequence SEQ ID No. 7, the gene therapy DNA vector VTvaf17-PLOD1 contains the coding region of PLOD1 therapeutic gene cloned to gene therapy DNA vector VTvaf17 with the nucleotide sequence SEQ ID No. 8.

Each of the constructed gene therapy DNA vectors, namely VTvaf17-COL1A1 or VTvaf17-COL1A2 or VTvaf17-P4HA1 or VTvaf17-P4HA2 or VTvaf17-COL7A1 or VTvaf17-CLCA2 or VTvaf17-ELN or VTvaf17-PLOD1 due to the limited size of VTvaf17 vector part not exceeding 3200 bp has the ability to efficiently penetrate into human and animal cells and express the COL1A1, or COL1A2, or P4HA1, or P4HA2, or COL7A1, or CLCA2, or ELN, or PLOD1 therapeutic gene cloned to it.

Each of the constructed gene therapy DNA vectors, namely VTvaf17-COL1A1, or VTvaf17-COL1A2, or VTvaf17-P4HA1, or VTvaf17-P4HA2, or VTvaf17-COL7A1, or VTvaf17-CLCA2, or VTvaf17-ELN, or VTvaf17-PLOD1 uses nucleotide sequences that are not antibiotic resistance genes, virus genes, or regulatory elements of viral genomes as the structure elements, which ensures its safe use for gene therapy in humans and animals.

A method of gene therapy DNA vector production based on gene therapy DNA vector VTvaf17 carrying the COL1A1, COL1A2, P4HA1, P4HA2, COL7A1, CLCA2,

ELN, PLOD1 therapeutic gene was also developed that involves obtaining each of gene therapy DNA vectors: VTvaf17-COL1A1 or VTvaf17-COL1A2 or VTvaf17-P4HA1 or VTvaf17-P4HA2 or VTvaf17-COL7A1 or VTvaf17-CLCA2 or VTvaf17-ELN or VTvaf17-PLOD1 as follows: the coding region of the COL1A1, or COL1A2, or P4HA1, or P4HA2, or COL7A1, or CLCA2, or ELN, or PLOD1 therapeutic gene is cloned to gene therapy DNA vector VTvaf17, and gene therapy DNA vector VTvaf17-COL1A1, SEQ ID No. 1, or VTvaf17-COL1A2, SEQ ID No. 2 or VTvaf17-P4HA1, SEQ ID No. 3, or VTvaf17-P4HA2, SEQ ID No. 4, or VTvaf17-COL7A1, SEQ ID No. 5, or VTvaf17-CLCA2, SEQ ID No. 6, or VTvaf17-ELN, SEQ ID No. 7, or VTvaf17-PLOD1, SEQ ID No. 8, respectively, is obtained, while the coding region of the COL1A1, or COL1A2, or P4HA1, or P4HA2, or COL7A1, or CLCA2, or ELN, or PLOD1 therapeutic gene is obtained by isolating total RNA from the human biological tissue sample followed by the reverse transcription reaction and PCR amplification using the obtained oligonucleotides and cleaving the amplification product by corresponding restriction endonucleases, while cloning to the gene therapy DNA vector VTvaf17 is performed by NheI and HindIII, or BamHI-KpnI, or BamHI-SalI, or SalI-EcoRI, or BamHI-EcoRI restriction sites, while the selection is performed without antibiotics,

at the same time, the following oligonucleotides produced for this purpose are used during gene therapy DNA vector VTvaf17-COL1A1, SEQ ID No. 1 production for the reverse transcription reaction and PCR amplification:

Col1A1_F
CCAGCTAGCGTCTAGGGTCTAGACATGTTC,
Col1A1_R
TATAAGCTTCTACAGGAAGCAGACAGGGCCAAC,

and the cleaving of amplification product and cloning of the coding region of COL1A1 gene to gene therapy DNA vector VTvaf17 is performed by NheI and HindIII restriction endonucleases, at the same time, the following oligonucleotides produced for this purpose are used during gene therapy DNA vector VTvaf17-COL1A2, SEQ ID No. 2 production for the reverse transcription reaction and PCR amplification:

Col1A2_F
CCAGCTAGCGTCTAAGTGCTAGACATGCTC,
Col1A2_R
CGAAGCTTTTATTTGAAACAGACTGGGCCA,

and the cleaving of amplification product and cloning of the coding region of COL1A2 gene to gene therapy DNA vector VTvaf17 is performed by NheI and HindIII restriction endonucleases,

at the same time, the following oligonucleotides produced for this purpose are used during gene therapy DNA vector VTvaf17-P4HA1, SEQ ID No. 3 production for the reverse transcription reaction and PCR amplification:

P4HA1_F
AGGATCCACCATGATCTGGTATATATTAATTATAGG,
P4HA1_R
TTCGGTACCTATTCCAATTCTGACAACGTACAAG,

and the cleaving of amplification product and cloning of the coding region of P4HA1 gene to gene therapy DNA vector VTvaf17 is performed by BamHI and KpnI restriction endonucleases,

at the same time, the following oligonucleotides produced for this purpose are used during gene therapy DNA vector VTvaf17-P4HA2, SEQ ID No. 4 production for the reverse transcription reaction and PCR amplification:

P4HA2_F
AGGATCCACCATGAAACTCTGGGTGTCTGCA,
P4HA2_R
CTTGTCGACTTAGTCAACTTCTGTTGATCCACA,

and the cleaving of amplification product and cloning of the coding region of P4HA2 gene to gene therapy DNA vector VTvaf17 is performed by BamHII and SalI restriction endonucleases,

at the same time, the following oligonucleotides produced for this purpose are used during gene therapy DNA vector VTvaf17-COL7A1, SEQ ID No. 5 production for the reverse transcription reaction and PCR amplification:

COL7A1_F
ATCGTCGACCACCATGACGCTGCGGCTTCTGGT,
COL7A1_R
ATAGAATTCAGTCCTGGGCAGTACCTGTC,

and the cleaving of amplification product and cloning of the coding region of COL7A1 gene to gene therapy DNA vector VTvaf17 is performed by SalI and EcoRI restriction endonucleases,

at the same time, the following oligonucleotides produced for this purpose are used during gene therapy DNA vector VTvaf17-CLCA2, SEQ ID No. 6 production for the reverse transcription reaction and PCR amplification:

CLCA2_F
AGGATCCACCATGACCCAAAGGAGCATTGC,
CLCA2_R
ATAGAATTCATAATAATTTTGTTCCATTCTCTTTC,

and the cleaving of amplification product and cloning of the coding region of CLCA2 gene to gene therapy DNA vector VTvaf17 is performed by BamHI and EcoRI restriction endonucleases,

at the same time, the following oligonucleotides produced for this purpose are used during gene therapy DNA vector VTvaf17-ELN, SEQ ID No. 7 production for the reverse transcription reaction and PCR amplification:

ELN_F
TTTGTCGACCACCATGGCGGGTCTGACGGCGG,
ELN_R
TTTTTGAATTCTCATTTTCTCTTCCGGCCACAAGCTT

and the cleaving of amplification product and cloning of the coding region of ELN gene to gene therapy DNA vector VTvaf17 is performed by SalI and EcoRI restriction endonucleases,

at the same time, the following oligonucleotides produced for this purpose are used during gene therapy DNA vector VTvaf17-PLOD1, SEQ ID No. 8 production for the reverse transcription reaction and PCR amplification:

PLOD1_F
GGATCCACCATGCGGCCCCTGCTGCTACT,
PLOD1_R
ATAGAATTCAGGGATCGACGAAGGAGACT,

and the cleaving of amplification product and cloning of the coding region of PLOD1 gene to gene therapy DNA vector VTvaf17 is performed by BamHII and EcoRI restriction endonucleases.

A method of use of the gene therapy DNA vector based on gene therapy DNA vector VTvaf17 carrying COL1A1, COL1A2, P4HA1, P4HA2, COL7A1, CLCA2, ELN, and PLOD1 therapeutic gene for treatment of diseases associated with disorders of formation of the extracellular matrix of the skin and other organs, skin structure, wound healing disorders, for prevention of skin ageing caused by internal and external factors, for treatment of hereditary and acquired connective tissue diseases, including Ehlers-Danlos syndrome, epidermolysis bullosa, Caffey disease, osteogenesis imperfecta, pathological scarring, formation of scars, superficial epidermal cysts, and hyperpigmentation was developed that involves transfection of the cells of patient or animal organs and tissues with the selected gene therapy DNA vector carrying the therapeutic gene based on gene therapy DNA vector VTvaf17, or several selected gene therapy DNA vectors carrying therapeutic genes based on gene therapy DNA vector VTvaf17, from the group of constructed gene therapy DNA vectors carrying therapeutic genes based on gene therapy DNA vector VTvaf17 and/or injection of autologous cells of said patient or animal transfected by the selected gene therapy DNA vector carrying therapeutic gene based on gene therapy DNA vector VTvaf17 or several selected gene therapy DNA vectors carrying the therapeutic genes based on gene therapy DNA vector VTvaf17 from the constructed gene therapy DNA vectors carrying therapeutic genes based on gene therapy DNA vector VTvaf17 into the organs and tissues of the same patient or animal and/or the injection of the selected gene therapy DNA vector carrying therapeutic gene based on gene therapy DNA vector VTvaf17 or several selected gene therapy DNA vectors carrying therapeutic genes based on gene therapy DNA vector VTvaf17 from the group of constructed gene therapy DNA vectors carrying therapeutic genes based on gene therapy DNA vector VTvaf17 into the organs and tissues of the same patient or animal, or the combination of the indicated methods.

A method of production of strain for construction of a gene therapy DNA vector for treatment of diseases associated with disorders of formation of the extracellular matrix of the skin and other organs, skin structure, wound healing disorders, for prevention of skin ageing caused by internal and external factors, for treatment of hereditary and acquired connective tissue diseases, including Ehlers-Danlos syndrome, epidermolysis bullosa, Caffey disease, osteogenesis imperfecta, pathological scarring, formation of scars, superficial epidermal cysts, and hyperpigmentation was developed that involves making electrocompetent cells of Escherichia coli strain SCS110-AF and subjecting these cells to electroporation with gene therapy DNA vector VTvaf17-COL1A1, or gene therapy DNA vector VTvaf17-COL1A2, or gene therapy DNA vector VTvaf17-P4HA1, or gene therapy DNA vector VTvaf17-P4HA2, or gene therapy DNA vector VTvaf17-COL7A1, or gene therapy DNA vector VTvaf17-CLCA2, or gene therapy DNA vector VTvaf17-ELN, or gene therapy DNA vector VTvaf17-PLOD1. After that, the cells are poured into agar plates (Petri dishes) with a selective medium containing yeastrel, peptone, 6% sucrose, and 10μg/ml of chloramphenicol, and as a result, Escherichia coli strain SCS110-AF/VTvaf17-COL1A1, or Escherichia coli strain SCS110-AF/VTvaf17-COL1A2, or Escherichia coli strain SCS110-AFNTvaf17-P4HA1, or Escherichia coli strain SCS110-AF/VTvaf17-P4HA2, or Escherichia coli strain SCS110-AF/VTvaf17-COL7A1, or Escherichia coli strain SCS110-AF/VTvaf17-CLCA2, or Escherichia coli strain SCS110-AF/VTvaf17-ELN, or Escherichia coli strain SCS110-AFNTvaf17-PLOD1 is obtained.

Escherichia coli strain SCS110-AF/VTvaf17-COL1A1 carrying the gene therapy DNA vector VTvaf17-COL1A1 for production thereof allowing for antibiotic-free selection during gene therapy DNA vector production, or Escherichia coli strain SCS110-AF/VTvaf17-COL1A2 carrying the gene therapy DNA vector VTvaf17-COL1A2 for production thereof allowing for antibiotic-free selection during gene therapy DNA vector production, or Escherichia coli strain SCS110-AFNTvaf17-P4HA1 carrying the gene therapy DNA vector VTvaf17-P4HA1 for production thereof allowing for antibiotic-free selection during gene therapy DNA vector production, or Escherichia coli strain SCS110-AF/VTvaf17-P4HA2 carrying the gene therapy DNA vector VTvaf17-P4HA2 for production thereof allowing for antibiotic-free selection during gene therapy DNA vector production, or Escherichia coli strain SCS110-AF/VTvaf17-COL7A1 carrying the gene therapy DNA vector VTvaf17-COL7A1 for production thereof allowing for antibiotic-free selection during gene therapy DNA vector production, or Escherichia coli strain SCS110-AF/VTvaf17-CLCA2 carrying the gene therapy DNA vector VTvaf17-CLCA2 for production thereof allowing for antibiotic-free selection during gene therapy DNA vector production, or Escherichia coli strain SCS110-AF/VTvaf17-ELN carrying the gene therapy DNA vector VTvaf17-ELN for production thereof allowing for antibiotic-free selection during gene therapy DNA vector production, or Escherichia coli strain SCS110-AF/VTvaf17-PLOD1 carrying the gene therapy DNA vector VTvaf17-PLOD1, for production thereof allowing for antibiotic-free selection during gene therapy DNA vector production is claimed for treatment of diseases associated with disorders of formation of the extracellular matrix of the skin and other organs, skin structure, wound healing disorders, for prevention of skin ageing caused by internal and external factors, for treatment of hereditary and acquired connective tissue diseases, including Ehlers-Danlos syndrome, epidermolysis bullosa, Caffey disease, osteogenesis imperfecta, pathological scarring, formation of scars, superficial epidermal cysts, and hyperpigmentation.

A method of production on an industrial scale of gene therapy DNA vector based on gene therapy DNA vector VTvaf17 carrying the COL1A1, or COL1A2, or P4HA1, or P4HA2, or COL7A1, or CLCA2, or ELN, or PLOD1 therapeutic gene for treatment of diseases associated with disorders of formation of the extracellular matrix of the skin and other organs, skin structure, wound healing disorders, for prevention of skin ageing caused by internal and external factors, for treatment of hereditary and acquired connective tissue diseases, including Ehlers-Danlos syndrome, epidermolysis bullosa, Caffey disease, osteogenesis imperfecta, pathological scarring, formation of scars, superficial epidermal cysts, and hyperpigmentation was developed that involves production of gene therapy DNA vector VTvaf17-COL1A1, or gene therapy DNA vector VTvaf17-COL1A2, or gene therapy DNA vector VTvaf17-P4HA1, or gene therapy DNA vector HK-BekTop VTvaf17-P4HA2, or gene therapy DNA vector VTvaf17-COL7A1, or gene therapy DNA vector VTvaf17-CLCA2, or gene therapy DNA vector VTvaf17-ELN, or gene therapy DNA vector VTvaf17-PLOD1 by inoculating a culture flask containing the prepared medium with seed culture selected from Escherichia coli strain SCS110-AFNTvaf17-COL 1A1, or Escherichia coli strain SCS110-AFNTvaf17-COL1A2, or Escherichia coli strain SCS110-AFNTvaf17-P4HA1, or Escherichia coli strain SCS110-AF/VTvaf17-P4HA2, or Escherichia coli strain SCS110-AFNTvaf17-COL7A1, or Escherichia coli strain SCS110-AFNTvaf17-CLCA2, or Escherichia coli strain SCS110-AFNTvaf17-ELN, or Escherichia coli strain SCS110-AF/VTvaf17-PLOD1, then the cell culture is incubated in an incubator shaker and transferred to an industrial fermenter, then grown to a stationary phase, then the fraction containing the target DNA product is extracted, multi-stage filtered, and purified by chromatographic methods.

The essence of the invention is explained in the drawings, where:

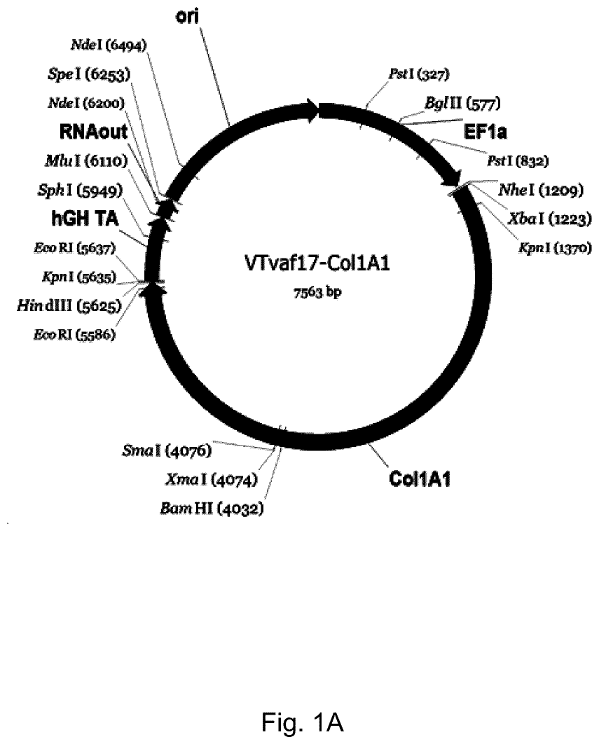

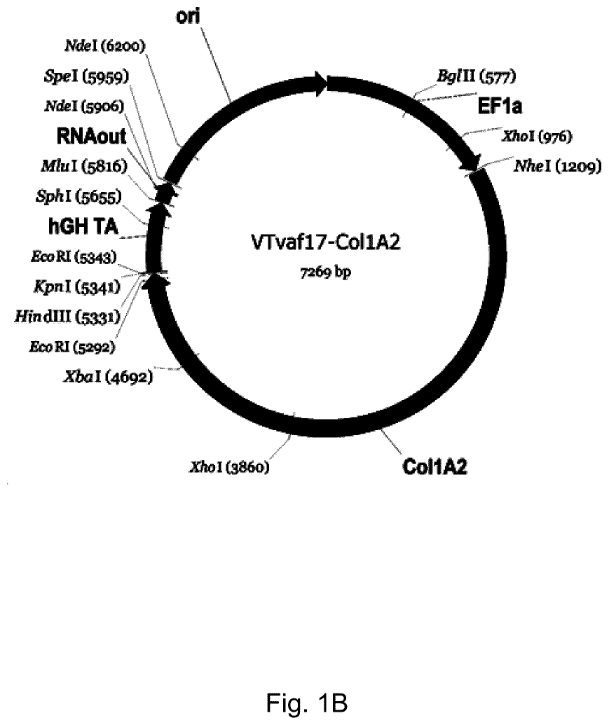

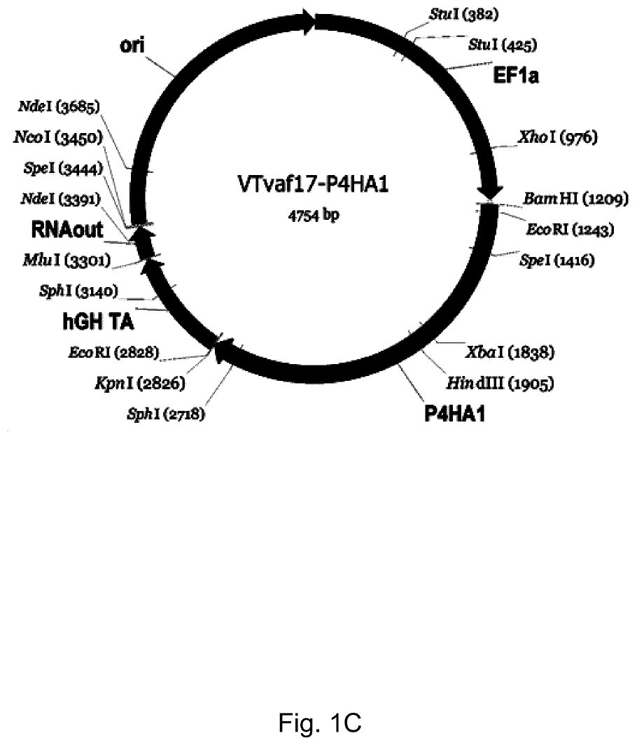

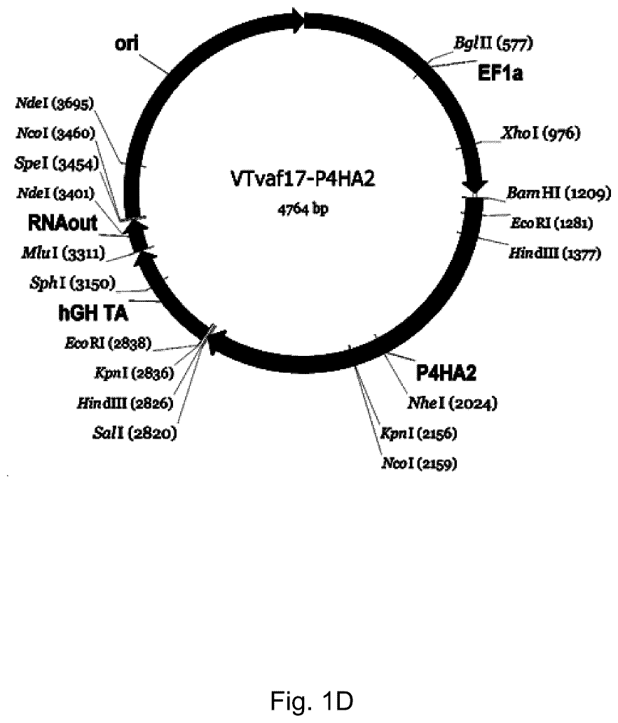

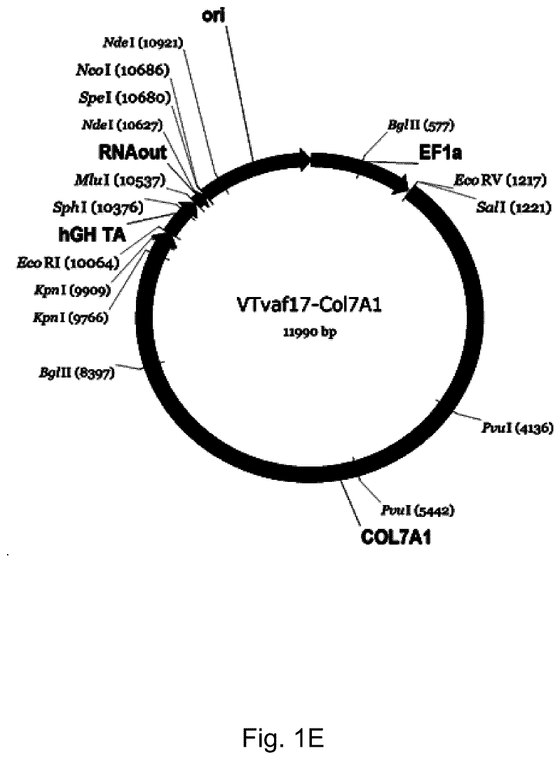

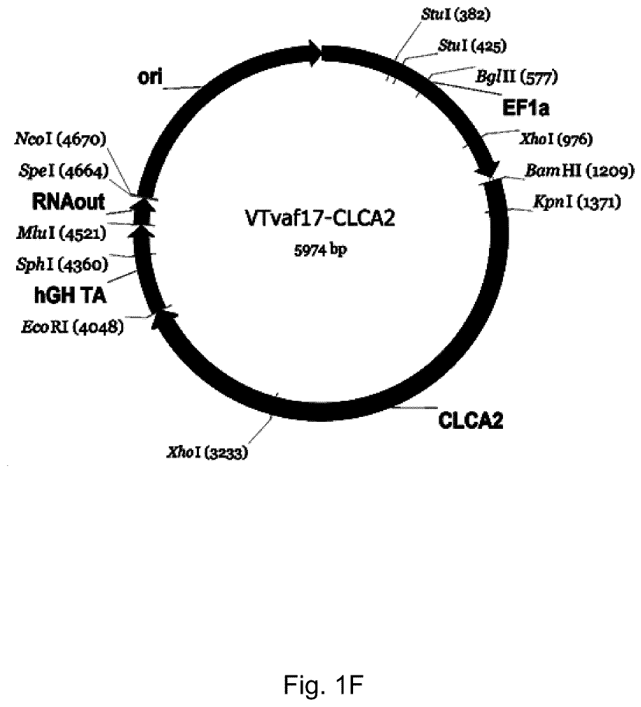

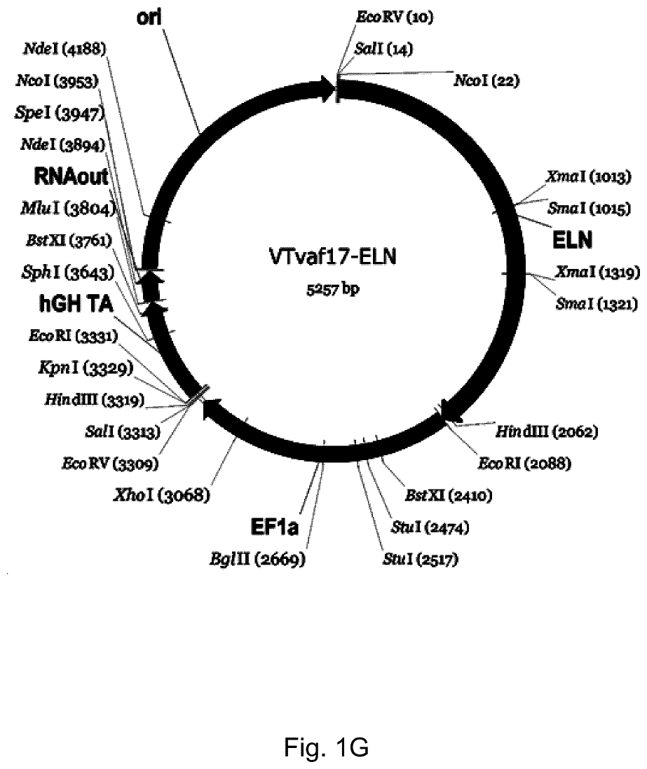

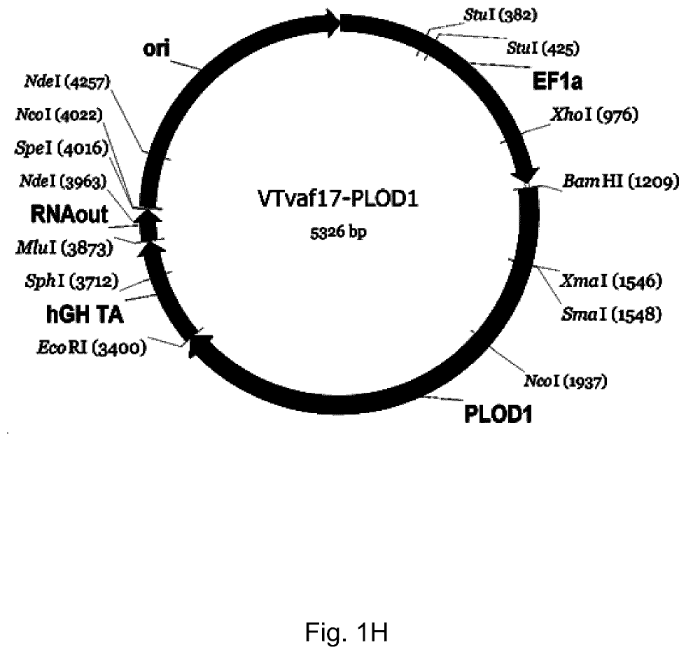

FIG. 1

shows the structure of gene therapy DNA vector VTvaf17 carrying the therapeutic gene selected from the group of COL1A1, COL1A2, P4HA1, P4HA2, COL7A1, CLCA2, ELN, and PLOD1 genes that constitutes a circular double-stranded DNA molecule capable of autonomous replication in Escherichia coli cells.

FIG. 1 shows the structures corresponding to:

A—gene therapy DNA vector VTvaf17-COL1A1,

B—gene therapy DNA vector VTvaf17-COL1A2,

C—gene therapy DNA vector VTvaf17-P4HA1,

D—gene therapy DNA vector VTvaf17-P4HA2,

E—gene therapy DNA vector VTvaf17-COL7A1,

F—gene therapy DNA vector VTvaf17-CLCA2,

G—gene therapy DNA vector VTvaf17-ELN,

H—gene therapy DNA vector VTvaf17-PLOD1.

The following structural elements of the vector are indicated in the structures:

EF1a—the promoter region of human elongation factor EF1A with an intrinsic enhancer contained in the first intron of the gene. It ensures efficient transcription of the recombinant gene in most human tissues,

The reading frame of the therapeutic gene corresponding to the coding region of the COL1A1 gene (FIG. 1A), COL1A2 gene (FIG. 1B), P4HA1 gene (FIG. 1C), P4HA2 gene (FIG. 1D), COL7A1 gene (FIG. 1E), CLCA2 gene (FIG. 1F), ELN gene (FIG. 1G), PLOD1 gene (FIG. 1H) respectively;

hGH TA—the transcription terminator and the polyadenylation site of the human growth factor gene,

ori—the origin of replication for autonomous replication with a single nucleotide substitution to increase plasmid production in the cells of most Escherichia coli strains,

RNA out—the regulatory element RNA-out of transposon Tn 10 allowing for antibiotic-free positive selection in case of the use of Escherichia coli strain SCS 110-AF.

Unique restriction sites are marked.

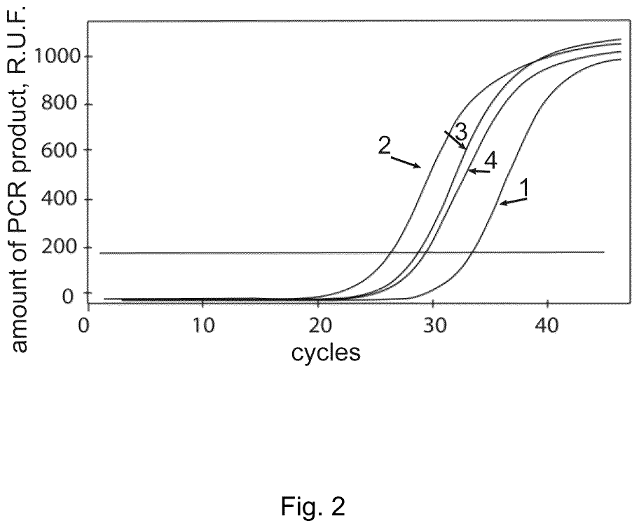

FIG. 2

shows diagrams of cDNA amplicon accumulation of the therapeutic gene, namely the COL1A1 gene, in HDFa primary human dermal fibroblast cell culture (ATCC PCS-201-01) before its transfection and 48 hours after transfection of these cells with gene therapy DNA vector VTvaf17-COL1A1 in order to assess the ability to penetrate into eukaryotic cells and functional activity, i.e. expression of the therapeutic gene at the mRNA level.

Curves of accumulation of amplicons during the reaction are shown in FIG. 2 corresponding to:

1—cDNA of COL1A1 gene in HDFa primary human dermal fibroblast cell culture before transfection with DNA vector VTvaf17-COL1A1,

2—cDNA of COL1A1 gene in HDFa primary human dermal fibroblast cell culture after transfection with DNA vector VTvaf17-COL1A1,

3—cDNA of B2M gene in HDFa primary human dermal fibroblast cell culture before transfection with DNA vector VTvaf17-COL1A1,

4—cDNA of B2M gene in HDFa primary human dermal fibroblast cell culture after transfection with DNA vector VTvaf17-COL1A1.

B2M (beta-2-microglobuline) gene listed in the GenBank database under number NM 004048.2 was used as a reference gene.

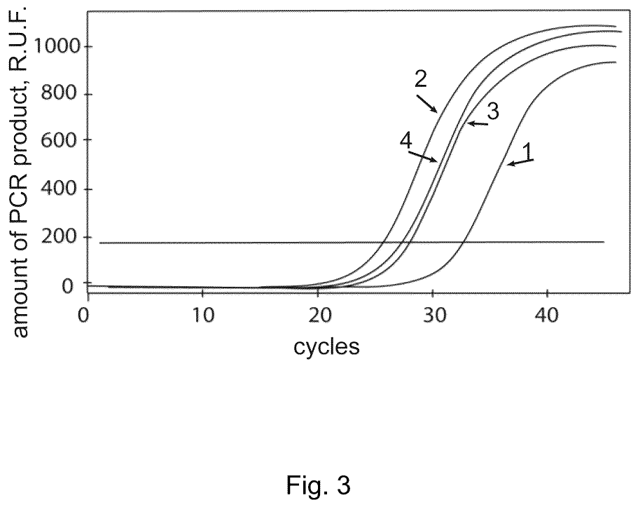

FIG. 3

shows diagrams of cDNA amplicon accumulation of the therapeutic gene, namely the COL1A2 gene, in HDFa primary human dermal fibroblast cell culture (ATCC PCS-201-01) before its transfection and 48 hours after transfection of these cells with gene therapy DNA vector VTvaf17-COL1A2 in order to assess the ability to penetrate into eukaryotic cells and functional activity, i.e. expression of the therapeutic gene at the mRNA level.

Curves of accumulation of amplicons during the reaction are shown in FIG. 3 corresponding to:

1—cDNA of COL1A2 gene in HDFa primary human dermal fibroblast cell culture before transfection with DNA vector VTvaf17-COL1A2,

2—cDNA of COL1A2 gene in HDFa primary human dermal fibroblast cell culture after transfection with DNA vector VTvaf17-COL1A2,

3—cDNA of B2M gene in HDFa primary human dermal fibroblast cell culture before transfection with DNA vector VTvaf17-COL1A2,

4—cDNA of B2M gene in HDFa primary human dermal fibroblast cell culture after transfection with DNA vector VTvaf17-COL1A2.

B2M (beta-2-microglobuline) gene listed in the GenBank database under number NM 004048.2 was used as a reference gene.

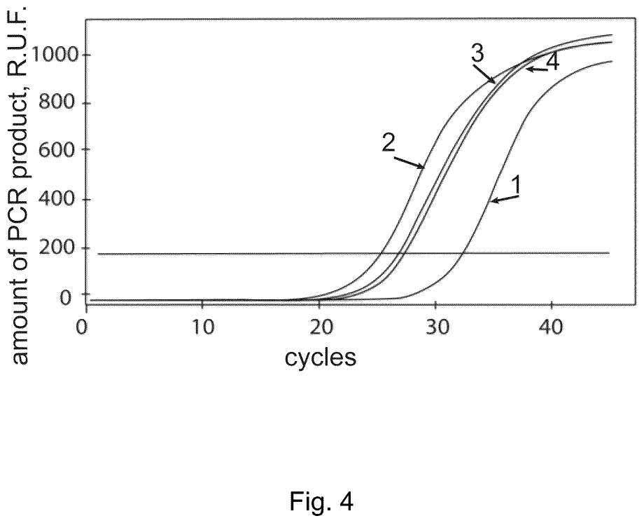

FIG. 4

shows diagrams of cDNA amplicon accumulation of the therapeutic gene, namely the P4HA1 gene, in Hs27 human primary foreskin fibroblast cell line (ATCC CRL-1634) before its transfection and 48 hours after transfection of these cells with gene therapy DNA vector VTvaf17-P4HA1 in order to assess the ability to penetrate into eukaryotic cells and functional activity, i.e. expression of the therapeutic gene at the mRNA level.

Curves of accumulation of amplicons during the reaction are shown in FIG. 4 corresponding to:

1—cDNA of P4HA1 gene in Hs27 human primary foreskin fibroblast cell line before transfection with DNA vector VTvaf17-P4HA1,

2—cDNA of P4HA1 gene in Hs27 human primary foreskin fibroblast cell line after transfection with DNA vector VTvaf17-P4HA1,

3—cDNA of B2M gene in Hs27 human primary foreskin fibroblast cell line before transfection with DNA vector VTvaf17-P4HA1,

4—cDNA of B2M gene in Hs27 human primary foreskin fibroblast cell line after transfection with DNA vector VTvaf17-P4HA1.

B2M (beta-2-microglobuline) gene listed in the GenBank database under number NM 004048.2 was used as a reference gene.

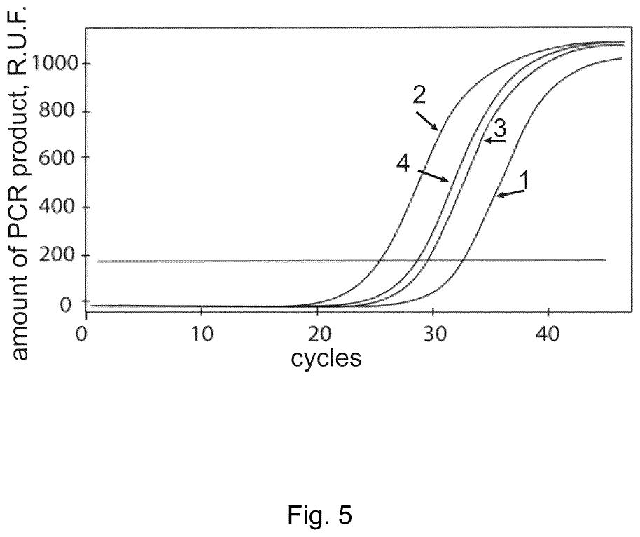

FIG. 5

shows diagrams of cDNA amplicon accumulation of the therapeutic gene, namely the P4HA2 gene, in Hs27 human primary foreskin fibroblast cell line (ATCC CRL-1634) before its transfection and 48 hours after transfection of these cells with gene therapy DNA vector VTvaf17-P4HA2 in order to assess the ability to penetrate into eukaryotic cells and functional activity, i.e. expression of the therapeutic gene at the mRNA level.

Curves of accumulation of amplicons during the reaction are shown in FIG. 5 corresponding to:

1—cDNA of P4HA2 gene in Hs27 human primary foreskin fibroblast cell line before transfection with DNA vector VTvaf17-P4HA2,

2—cDNA of P4HA2 gene in Hs27 human primary foreskin fibroblast cell line after transfection with DNA vector VTvaf17-P4HA2,

3—cDNA of B2M gene in Hs27 human primary foreskin fibroblast cell line before transfection with DNA vector VTvaf17-P4HA2,

4—cDNA of B2M gene in Hs27 human primary foreskin fibroblast cell line after transfection with DNA vector VTvaf17-P4HA2.

B2M (beta-2-microglobuline) gene listed in the GenBank database under number NM 004048.2 was used as a reference gene.

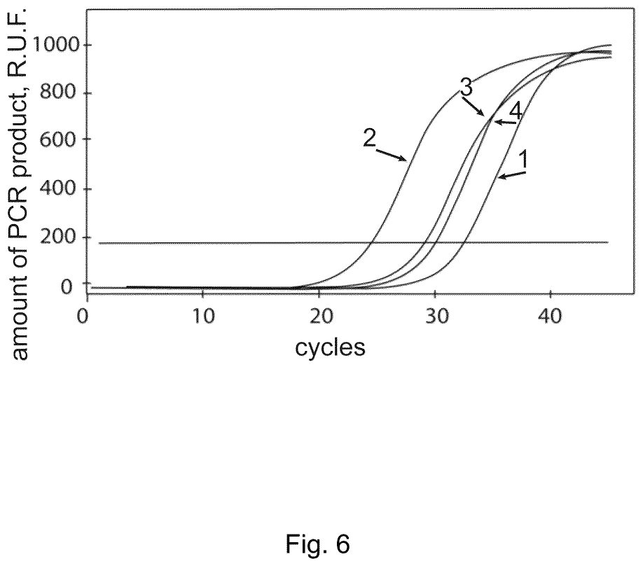

FIG. 6

shows diagrams of cDNA amplicon accumulation of the therapeutic gene, namely the COL7A1 gene, in HT 297.T fibroblast culture (ATCC® CRL-7782™) before its transfection and 48 hours after transfection of these cells with gene therapy DNA vector VTvaf17-COL7A1 in order to assess the ability to penetrate into eukaryotic cells and functional activity, i.e. expression of the therapeutic gene at the mRNA level.

Curves of accumulation of amplicons during the reaction are shown in FIG. 6 corresponding to:

1—cDNA of COL7A1 gene in HT 297.T primary fibroblast culture before transfection with DNA vector VTvaf17-COL7A1,

2—cDNA of COL7A1 gene in HT 297.T primary fibroblast culture after transfection with DNA vector VTvaf17-COL7A1,

3—cDNA of B2M gene in HT 297.T primary fibroblast culture before transfection with DNA vector VTvaf17-COL7A1,

4—cDNA of B2M gene in HT 297.T primary fibroblast culture after transfection with DNA vector VTvaf17-COL7A1.

B2M (beta-2-microglobuline) gene listed in the GenBank database under number NM 004048.2 was used as a reference gene.

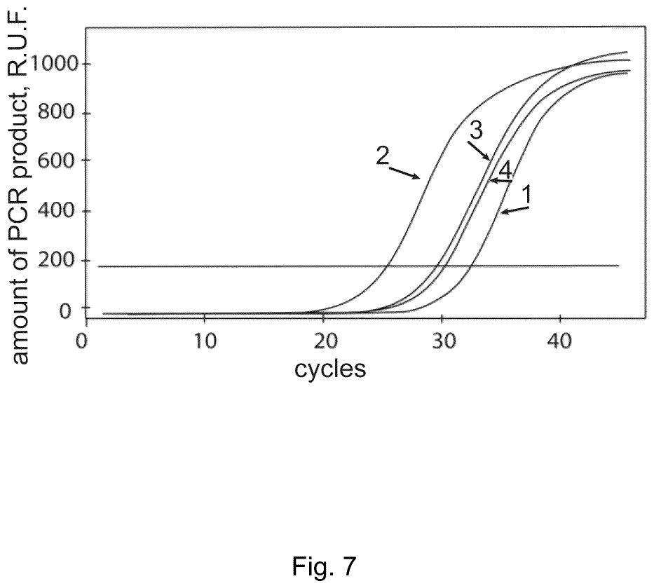

FIG. 7

shows diagrams of cDNA amplicon accumulation of the therapeutic gene, namely the CLCA2 gene, in HT 297.T fibroblast culture (ATCC® CRL-7782™) before its transfection and 48 hours after transfection of these cells with gene therapy DNA vector VTvaf17-CLCA2 in order to assess the ability to penetrate into eukaryotic cells and functional activity, i.e. expression of the therapeutic gene at the mRNA level.

Curves of accumulation of amplicons during the reaction are shown in FIG. 7 corresponding to:

1—cDNA of CLCA2 gene in HT 297.T primary fibroblast culture before transfection with DNA vector VTvaf17-CLCA2,

2—cDNA of CLCA2 gene in HT 297.T primary fibroblast culture after transfection with DNA vector VTvaf17-CLCA2,

3—cDNA of B2M gene in HT 297.T primary fibroblast culture before transfection with DNA vector VTvaf17-CLCA2,

4—cDNA of B2M gene in HT 297.T primary fibroblast culture after transfection with DNA vector VTvaf17-CLCA2.

B2M (beta-2-microglobuline) gene listed in the GenBank database under number NM 004048.2 was used as a reference gene.

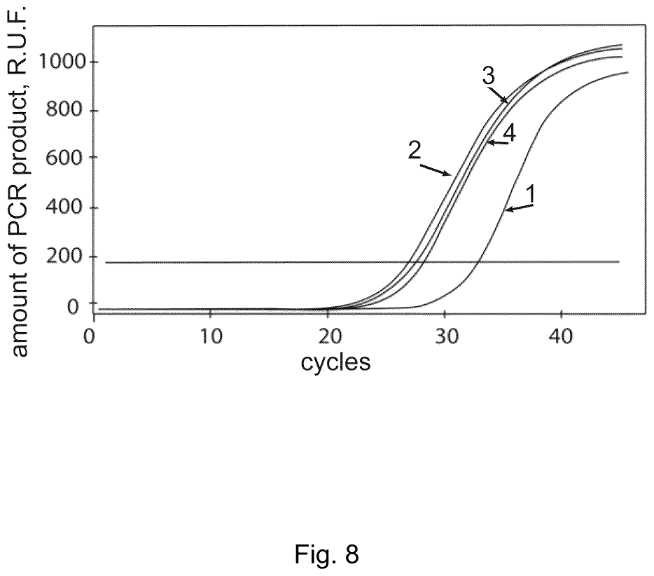

FIG. 8

shows diagrams of cDNA amplicon accumulation of the therapeutic gene, namely the ELN gene, in HEKa primary human epidermal keratinocyte cell culture (ATCC PCS-200-011) before its transfection and 48 hours after transfection of these cells with gene therapy DNA vector VTvaf17-ELN in order to assess the ability to penetrate into eukaryotic cells and functional activity, i.e. expression of the therapeutic gene at the mRNA level.

Curves of accumulation of amplicons during the reaction are shown in FIG. 8 corresponding to:

1—cDNA of ELN gene in HEKa primary human epidermal keratinocyte cell culture before transfection with DNA vector VTvaf17-ELN,

2—cDNA of ELN gene in HEKa primary human epidermal keratinocyte cell culture after transfection with DNA vector VTvaf17-ELN,

3—cDNA of B2M gene in HEKa primary human epidermal keratinocyte cell culture before transfection with DNA vector VTvaf17-ELN,

4—cDNA of B2M gene in HEKa primary human epidermal keratinocyte cell culture after transfection with DNA vector VTvaf17-ELN.

B2M (beta-2-microglobuline) gene listed in the GenBank database under number NM 004048.2 was used as a reference gene.

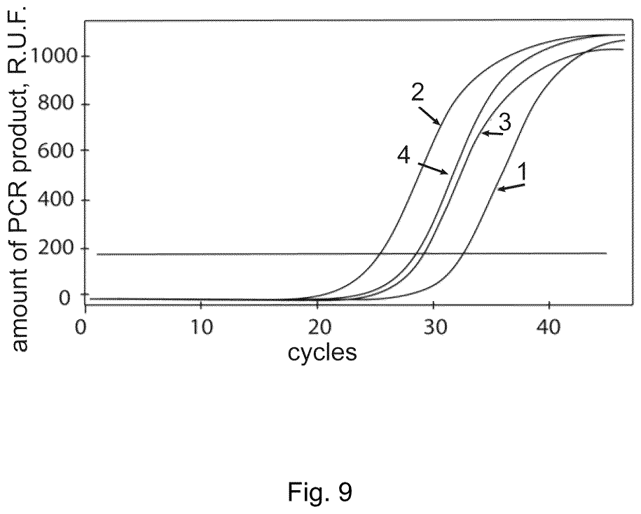

FIG. 9

shows diagrams of cDNA amplicon accumulation of the therapeutic gene, namely the PLOD1 gene, in HEMa primary human epidermal melanocyte cell culture (ATCC® PCS200013™) before its transfection and 48 hours after transfection of these cells with gene therapy DNA vector VTvaf17-PLOD1 in order to assess the ability to penetrate into eukaryotic cells and functional activity, i.e. expression of the therapeutic gene at the mRNA level.

Curves of accumulation of amplicons during the reaction are shown in FIG. 9

1—cDNA of PLOD1 gene in HEMa primary human epidermal melanocyte cell culture before transfection with DNA vector VTvaf17-PLOD1,

2—cDNA of PLOD1 gene in HEMa primary human epidermal melanocyte cell culture after transfection with DNA vector VTvaf17-PLOD1,

3—cDNA of B2M gene in HEMa primary human epidermal melanocyte cell culture before transfection with DNA vector VTvaf17-PLOD1,

4—cDNA of B2M gene in HEMa primary human epidermal melanocyte cell culture before transfection with DNA vector VTvaf17-PLOD1.

B2M (beta-2-microglobuline) gene listed in the GenBank database under number NM 004048.2 was used as a reference gene.

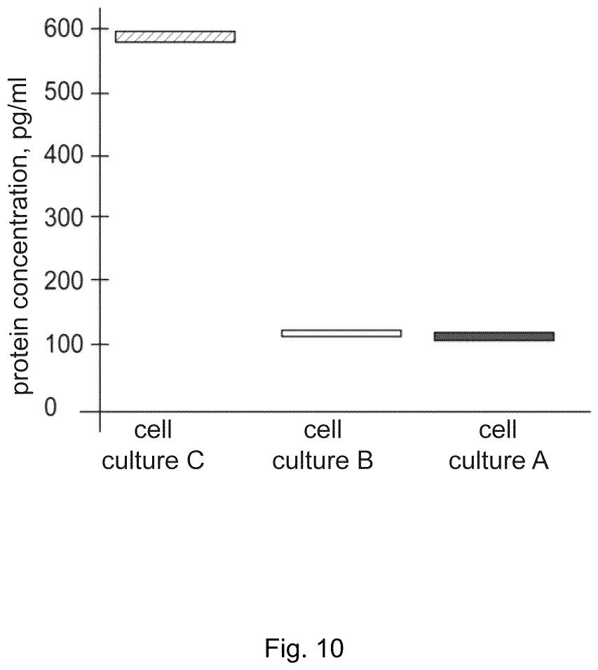

FIG. 10

shows the plot of COL1A1 protein concentration in the cell lysate of HDFa primary human dermal fibroblasts (ATCC PCS-201-01) after transfection of these cells with DNA vector VTvaf17-COL1A1 in order to assess the functional activity, i.e. expression at the protein level based on the COL1A1 protein concentration change in the cell lysate.

The following elements are indicated in FIG. 10:

culture A—HDFa human dermal fibroblast cell culture transfected with aqueous dendrimer solution without plasmid DNA (reference),

culture B—HDFa human dermal fibroblast cell culture transfected with DNA vector VTvaf17,

culture C—HDFa human dermal fibroblast cell culture transfected with DNA vector VTvaf17-COL1A1.

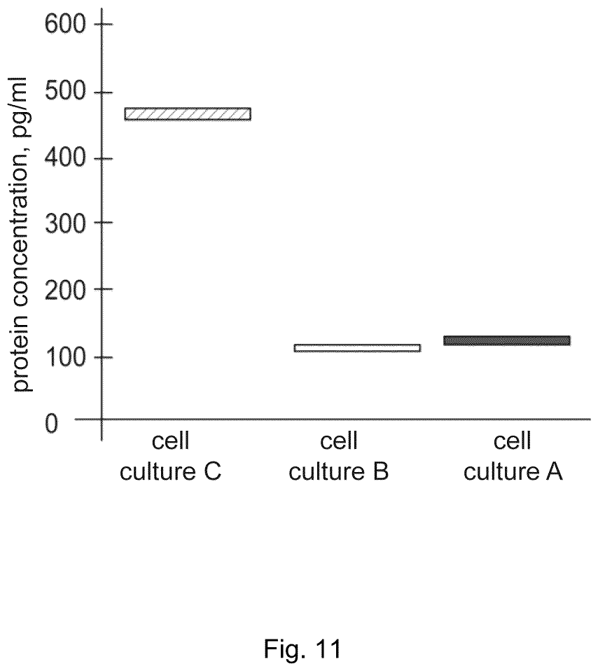

FIG. 11

shows the plot of COL1A2 protein concentration in the lysate of HDFa primary human dermal fibroblast cell culture (ATCC PCS-201-01) after transfection of these cells with gene therapy DNA vector VTvaf17-COL1A2 in order to assess the functional activity, i.e. the therapeutic gene expression at the protein level, and the possibility of increasing the level of protein expression by gene therapy DNA vector based on gene therapy vector VTvaf17 carrying the COL1A2 therapeutic gene.

The following elements are indicated in FIG. 11:

culture A—HDFa human dermal fibroblast cell culture transfected with aqueous dendrimer solution without plasmid DNA (reference),

culture B—HDFa human primary dermal fibroblast cell culture transfected with DNA vector VTvaf17,

culture C—HDFa human primary dermal fibroblast cell culture transfected with DNA vector VTvaf17-COL1A2.

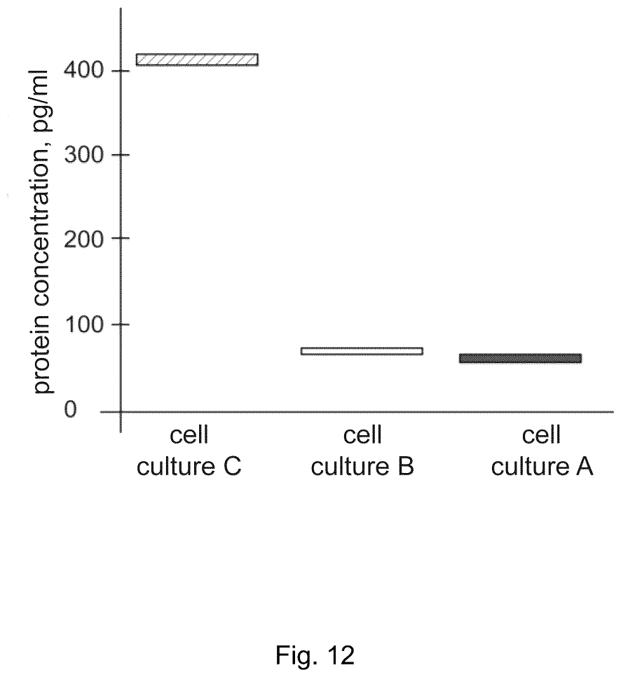

FIG. 12

shows the plot of P4HA1 protein concentration in the lysate of Hs27 human primary foreskin fibroblast cell line (ATCC CRL-1634) after transfection of these cells with DNA vector VTvaf17-P4HA1 in order to assess the functional activity, i.e. the therapeutic gene expression at the protein level, and the possibility of increasing the level of protein expression by gene therapy DNA vector based on gene therapy DNA vector VTvaf17 carrying the P4HA1 therapeutic gene.

The following elements are indicated in FIG. 12:

culture A—Hs27 human foreskin fibroblast cell culture transfected with aqueous dendrimer solution without plasmid DNA (reference),

culture B—Hs27 human foreskin fibroblast cell culture transfected with DNA vector VTvaf17,

culture C—Hs27 human foreskin fibroblast cell culture transfected with DNA vector VTvaf17-P4HA1.

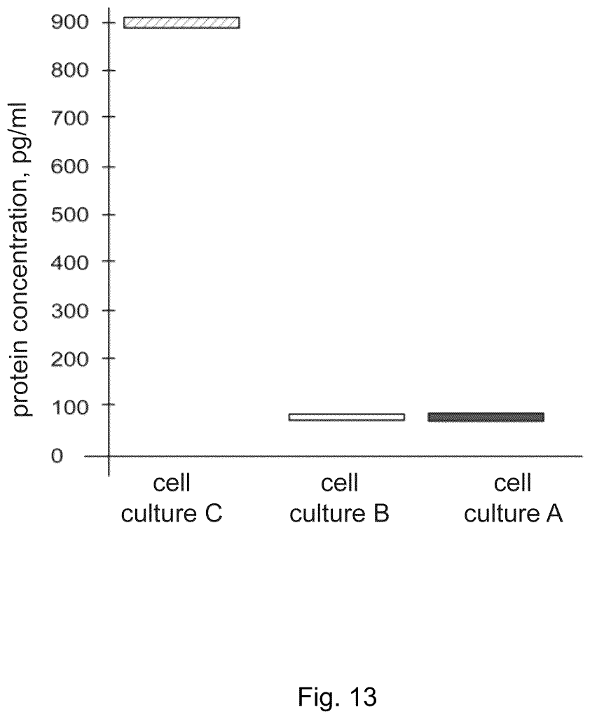

FIG. 13

shows the plot of P4HA2 protein concentration in the lysate of Hs27 human primary foreskin fibroblast cell line (ATCC CRL-1634) after transfection of these cells with DNA vector VTvaf17-P4HA2 in order to assess the functional activity, i.e. the therapeutic gene expression at the protein level, and the possibility of increasing the level of protein expression by gene therapy DNA vector based on gene therapy DNA vector VTvaf17 carrying the P4HA2 therapeutic gene.

The following elements are indicated in FIG. 13:

culture A—Hs27 human foreskin fibroblast cell culture transfected with aqueous dendrimer solution without plasmid DNA (reference),

culture B—Hs27 human foreskin fibroblast cell culture transfected with DNA vector VTvaf17,

culture C—Hs27 human foreskin fibroblast cell culture transfected with DNA vector VTvaf17-P4HA2.

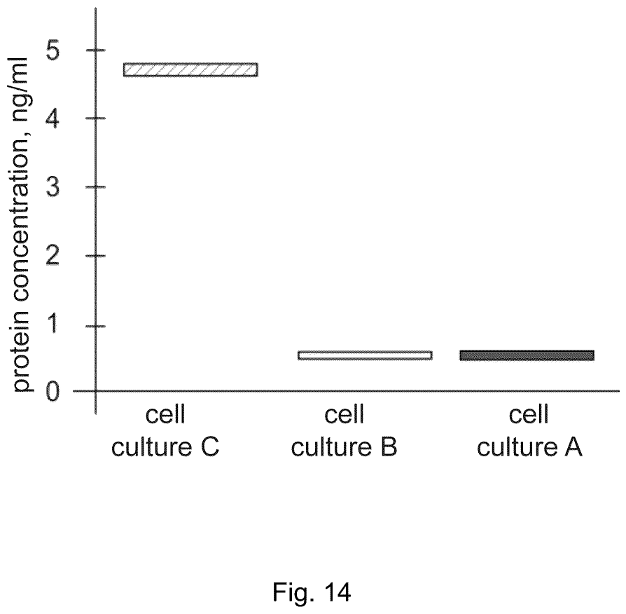

FIG. 14

shows the plot of COL7A1 protein concentration in the cell lysate of HT 297.T human fibroblasts (ATCC® CRL-7782™) after transfection of these cells with DNA vector VTvaf17-COL7A1 in order to assess the functional activity, i.e. expression at the protein level based on the COL7A1 protein concentration change in the cell lysate.

The following elements are indicated in FIG. 14:

culture A—HT 297.T human fibroblast cell culture transfected with aqueous dendrimer solution without plasmid DNA (reference),

culture B—HT 297.T human fibroblast cell culture transfected with DNA vector VTvaf17,

culture C—HT 297.T human fibroblast cell culture transfected with DNA vector VTvaf17-COL7A1.

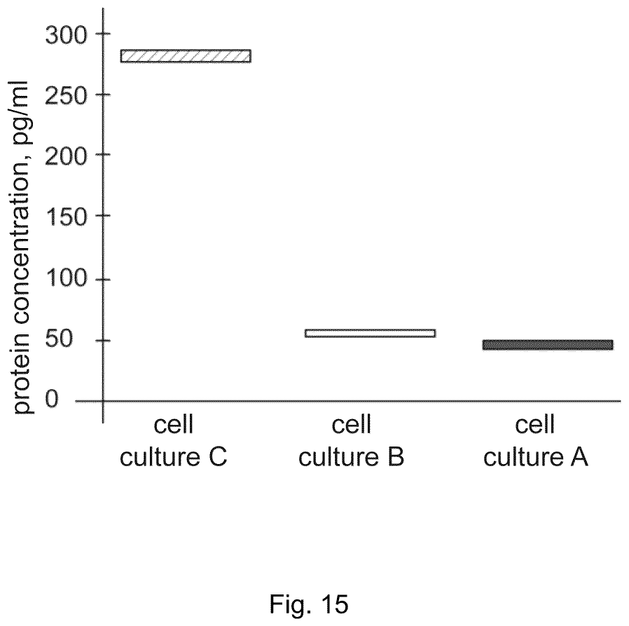

FIG. 15 p shows the plot of CLCA2 protein concentration in the cell lysate of HT 297.T human fibroblasts (ATCC® CRL-7782™) after transfection of these cells with DNA vector VTvaf17-CLCA2 in order to assess the functional activity, i.e. expression at the protein level based on the CLCA2 protein concentration change in the cell lysate.

The following elements are indicated in FIG. 15:

culture A—HT 297.T human fibroblast cell culture transfected with aqueous dendrimer solution without plasmid DNA (reference),

culture B—HT 297.T human fibroblast cell culture transfected with DNA vector VTvaf17,

culture C—HT 297.T human fibroblast cell culture transfected with DNA vector VTvaf17-CLCA2.

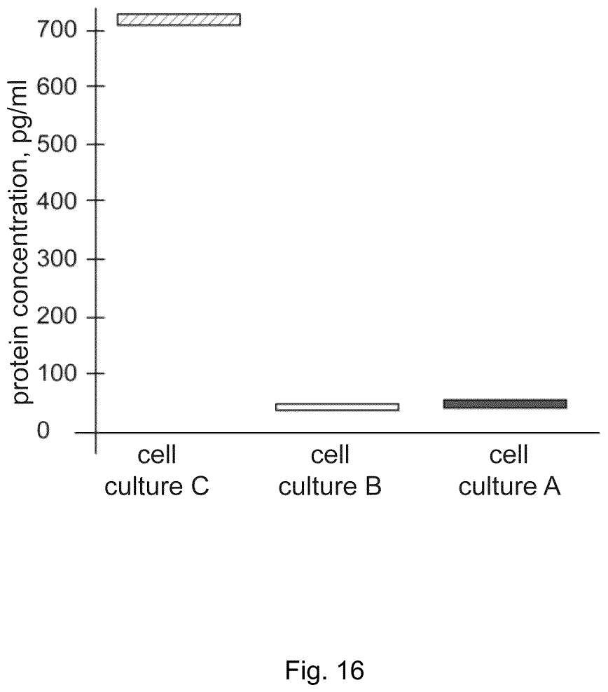

FIG. 16

shows the plot of ELN protein concentration in the cell lysate of HEKa human epidermal keratinocyte cell culture (ATCC PCS-200-011) after transfection of these cells with DNA vector VTvaf17-ELN in order to assess the functional activity, i.e. expression at the protein level based on the ELN protein concentration change in the cell lysate.

The following elements are indicated in FIG. 16:

culture A—HEKa human epidermal keratinocyte cell culture transfected with aqueous dendrimer solution without plasmid DNA (reference),

culture B—HEKa human epidermal keratinocyte cell culture transfected with DNA vector VTvaf17,

culture C—HEKa human epidermal keratinocyte cell culture transfected with DNA vector VTvaf17-ELN.

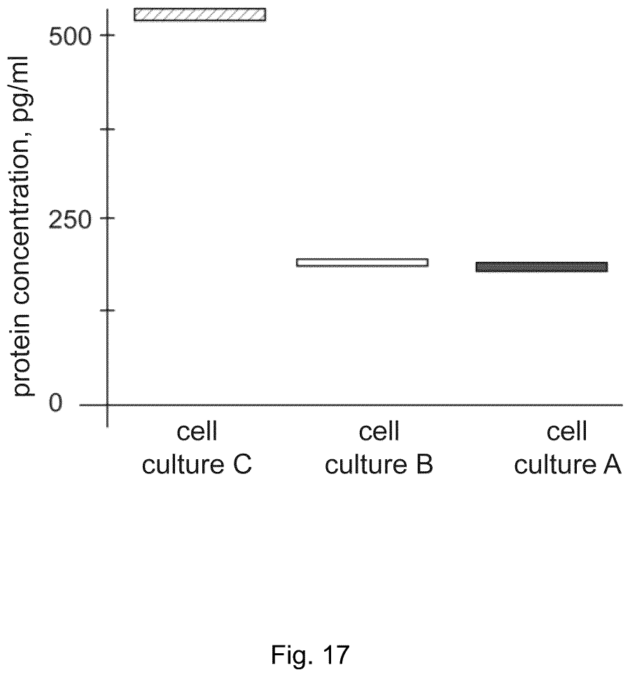

FIG. 17

shows the plot of PLOD1 protein concentration in the cell lysate of HEMa primary human epidermal melanocyte cell culture (ATCC® PCS200-013™) after transfection of these cells with DNA vector VTvaf17-PLOD1 in order to assess the functional activity, i.e. expression at the protein level based on the PLOD1 protein concentration change in the cell lysate.

The following elements are indicated in FIG. 17:

culture A—HEMa primary human epidermal melanocyte cell culture transfected with aqueous dendrimer solution without plasmid DNA (reference),

culture B—HEMa human epidermal melanocyte cell culture transfected with

DNA vector VTvaf17,

culture C—HEMa human epidermal melanocyte cell culture transfected with DNA vector VTvaf17-PLOD1.

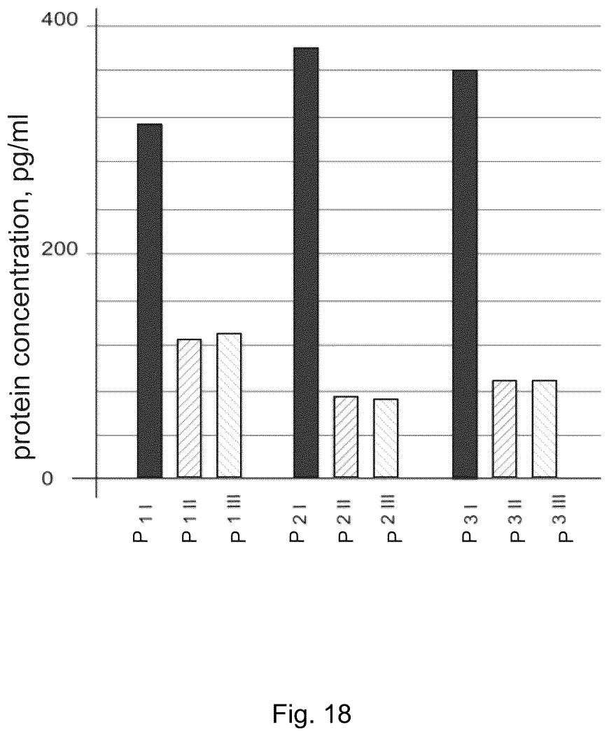

FIG. 18

shows the plot of P4HA2 protein concentration in the skin biopsy samples of three patients after injection of gene therapy DNA vector VTvaf17-P4HA2 into the skin of these patients in order to assess the functional activity, i.e. the expression of the therapeutic gene at the protein level, and the possibility of increasing the level of protein expression using gene therapy DNA vector based on gene therapy DNA vector VTvaf17 carrying the P4HA2 therapeutic gene.

The following elements are indicated in FIG. 18:

P1I—patient P1 skin biopsy in the region of injection of gene therapy DNA vector VTvaf17-P4HA2,

P1 II—patient P1 skin biopsy in the region of injection of gene therapy DNA vector VTvaf17 (placebo),

P1 III—patient P1 skin biopsy from intact site,

P2I—patient P2 skin biopsy in the region of injection of gene therapy DNA vector VTvaf17-P4HA2,

P2II—patient P2 skin biopsy in the region of injection of gene therapy DNA vector VTvaf17 (placebo),

P2III—patient P2 skin biopsy from intact site,

P3 I—patient P3 skin biopsy in the region of injection of gene therapy DNA vector VTvaf17-P4HA2,

P3 II—patient P3 skin biopsy in the region of injection of gene therapy DNA vector VTvaf17 (placebo),

P3III—patient P3 skin biopsy from intact site.

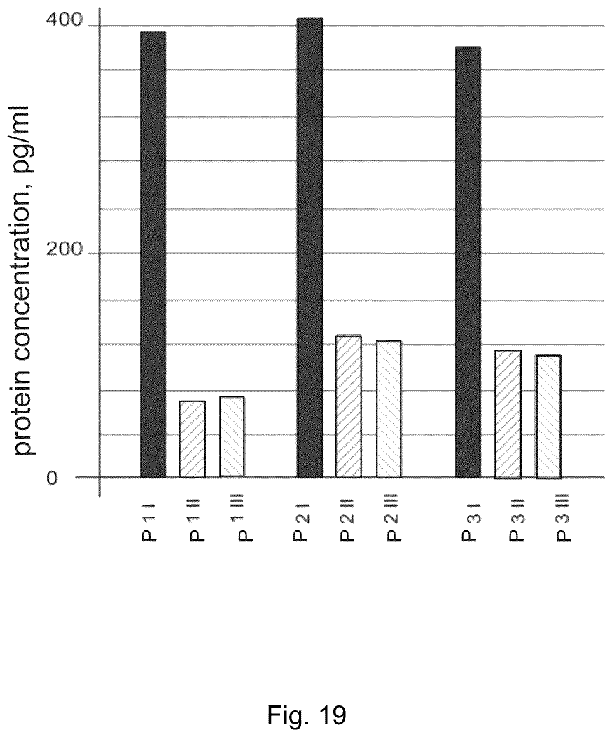

FIG. 19

shows the plot of P4HA1 protein concentration in the skin biopsy samples of three patients after injection of gene therapy DNA vector VTvaf17-P4HA1 into the skin of these patients in order to assess the functional activity, i.e. the expression of the therapeutic gene at the protein level, and the possibility of increasing the level of protein expression using gene therapy DNA vector based on gene therapy DNA vector VTvaf17 carrying the P4HA1 therapeutic gene.

The following elements are indicated in FIG. 19:

P1I—patient P1 skin biopsy in the region of injection of gene therapy DNA vector VTvaf17-P4HA1,

P1II—patient P1 skin biopsy in the region of injection of gene therapy DNA vector VTvaf17 (placebo),

P1III—patient P1 skin biopsy from intact site,

P2I—patient P2 skin biopsy in the region of injection of gene therapy DNA vector VTvaf17-P4HA1,

P2II—patient P2 skin biopsy in the region of injection of gene therapy DNA vector VTvaf17 (placebo),

P2III—patient P2 skin biopsy from intact site,

P3I—patient P3 skin biopsy in the region of injection of gene therapy DNA vector VTvaf17-P4HA1,

P3II—patient P3 skin biopsy in the region of injection of gene therapy DNA vector VTvaf17 (placebo),

P3III—patient P3 skin biopsy from intact site.

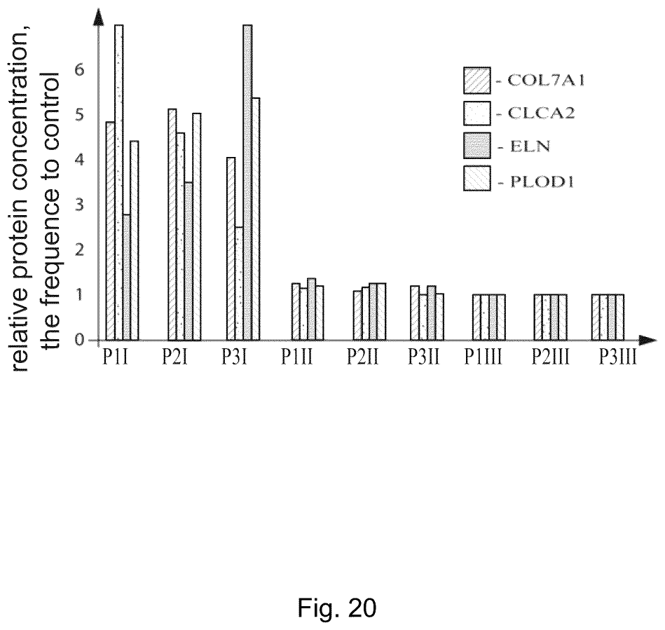

FIG. 20

shows the plot of COL7A1, CLCA2, ELN and PLOD1 protein concentration in the skin biopsy specimens of three patients after combined injection of gene therapy DNA vector VTvaf17-COL7A1, gene therapy DNA vector VTvaf17-CLCA2, gene therapy DNA vector VTvaf17-ELN, and gene therapy DNA vector VTvaf17-PLOD1 into the skin of these patients in order to assess the functional activity, i.e. the expression of the therapeutic gene at the protein level, and the possibility of increasing the level of protein expression using gene therapy DNA vectors based on gene therapy DNA vector VTvaf17 carrying the COL7A1, CLCA2, ELN, and PLOD1 therapeutic gene.

The following elements are indicated in FIG. 20:

P1I—patient P1 skin biopsy in the region of injection of a mixture of gene therapy DNA vector VTvaf17-COL7A1, gene therapy DNA vector VTvaf17-CLCA2, gene therapy DNA vector VTvaf17-ELN, and gene therapy DNA vector VTvaf17-PLOD1,

P1II—patient P 1 skin biopsy in the region of injection of gene therapy DNA vector VTvaf17 (placebo),

P1III—patient P1 skin biopsy from intact site,

P2I—patient P2 skin biopsy in the region of injection of a mixture of gene therapy DNA vector VTvaf17-COL7A1, gene therapy DNA vector VTvaf17-CLCA2, gene therapy DNA vector VTvaf17-ELN, and gene therapy DNA vector VTvaf17-PLOD1,

P2II—patient P2 skin biopsy in the region of injection of gene therapy DNA vector VTvaf17 (placebo),

P2III—patient P2 skin biopsy from intact site,

P3I—patient P3 skin biopsy in the region of injection of a mixture of gene therapy DNA vector VTvaf17-COL7A1, gene therapy DNA vector VTvaf17-CLCA2, gene therapy DNA vector VTvaf17-ELN, gene therapy DNA vector VTvaf17-PLOD1,

P3II—patient P3 skin biopsy in the region of injection of gene therapy DNA vector VTvaf17 (placebo),

P3III—patient P3 skin biopsy from intact site.

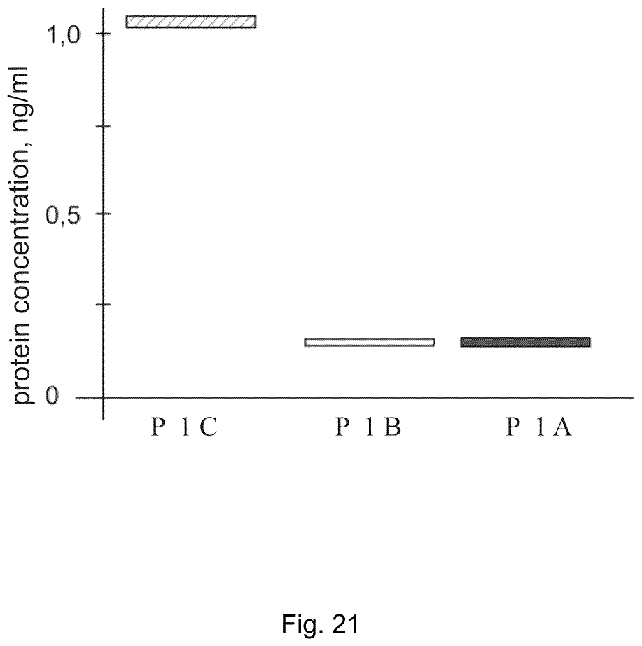

FIG. 21

shows the plot of COL1A2 protein concentration in human skin biopsy samples after subcutaneous injection of autologous fibroblast cell culture transfected with the gene therapy DNA vector VTvaf17-COL1A2 in order to demonstrate the method of use by injecting autologous cells transfected with the gene therapy DNA vector VTvaf17-COL1A2.

The following elements are indicated in FIG. 21:

P1C—patient P1 skin biopsy in the region of injection of autologous fibroblast culture of the patient transfected with gene therapy DNA vector VTvaf17-COL1A2,

P1B—patient P1 skin biopsy in the region of injection of autologous fibroblasts of the patient transfected with gene therapy DNA vector VTvaf17,

P1A—patient P1 skin biopsy from intact site.

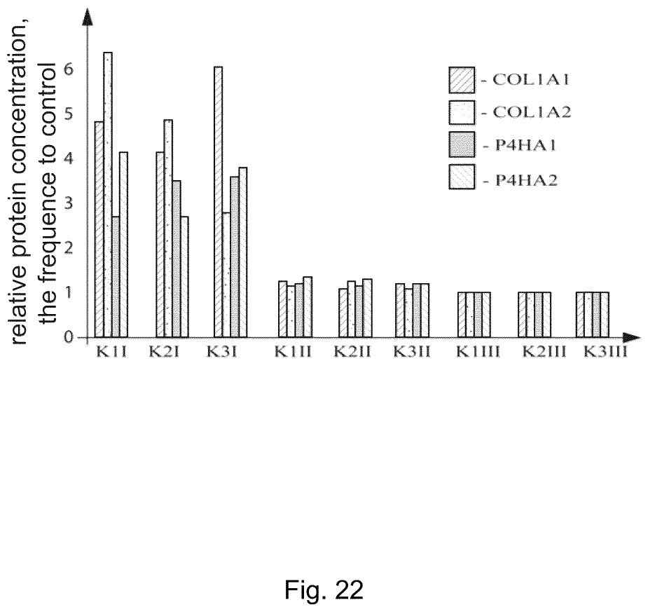

FIG. 22

shows the plot of COL1A1, COL1A2, P4HA1, and P4HA2 protein concentration in the skin biopsy samples of three rats after the combined injection in the skin of these animals with the following gene therapy DNA vectors: VTvaf17-COL1A1, VTvaf17-COL1A2, VTvaf17-P4HA1, and VTvaf17-P4HA2 in order to assess their functional activity, i.e. the therapeutic gene expression at the protein level, and the possibility of increasing the level of protein expression by gene therapy DNA vectors based on gene therapy vector VTvaf17 carrying the COL1A1, COL1A2, P4HA1, and P4HA2 therapeutic gene.

The following elements are indicated in FIG. 22:

K1I—rat K1 skin biopsy sample in the region of injection of a mixture of gene therapy DNA vectors: VTvaf17-COL1A1, VTvaf17-COL1A2, VTvaf17-P4HA1, and VTvaf17-P4HA2,

K1II—rat K1 skin biopsy sample in the region of injection of gene therapy DNA vector VTvaf17 (placebo),

K1III—rat K1 skin biopsy sample of the reference intact site,

K2I—rat K2 skin biopsy sample in the region of injection of a mixture of gene therapy DNA vectors: VTvaf17-COL1 A1, VTvaf17-COL1A2, VTvaf17-P4HA1 and VTvaf17-P4HA2,

K2II—rat K2 skin biopsy sample in the region of injection of gene therapy DNA vector VTvaf17 (placebo),

K2III—rat K2 skin biopsy sample of the reference intact site,

K3I—rat K3 skin biopsy sample in the region of injection of a mixture of gene therapy DNA vectors: VTvaf17-COL1A1, VTvaf17-COL1A2, VTvaf17 -P4HA1, and VTvaf17-P4HA2,

K3II—rat K3 skin biopsy sample in the region of injection of gene therapy DNA vector VTvaf17 (placebo),

K3III—rat K3 skin biopsy sample of the reference intact site.

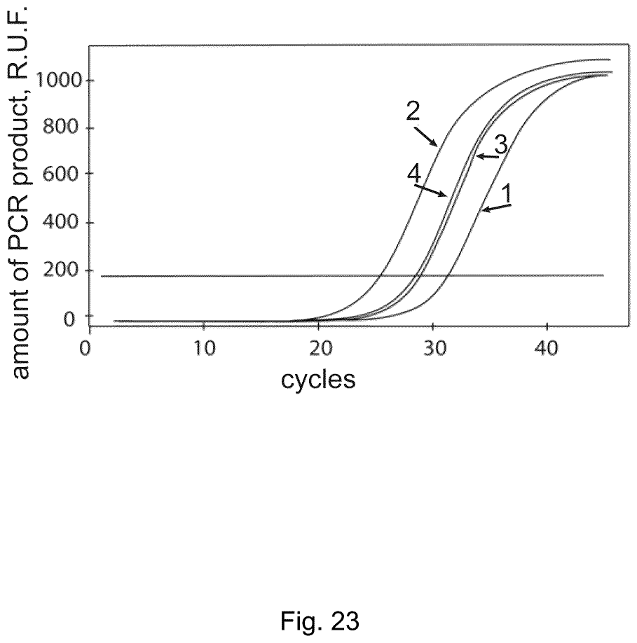

FIG. 23

shows diagrams of cDNA amplicon accumulation of the ELN therapeutic gene in bovine dermal fibroblast cells (ScienCell, Cat. #B2300) before and 48 hours after transfection of these cells with the DNA vector VTvaf17-ELN in order to demonstrate the method of use by injecting the gene therapy DNA vector in animals.

Curves of accumulation of amplicons during the reaction are shown in FIG. 23 corresponding to:

1—cDNA of ELN gene in bovine dermal fibroblast cells before transfection with gene therapy DNA vector VTvaf17-ELN,

2—cDNA of ELN gene in bovine dermal fibroblast cells after transfection with gene therapy DNA vector VTvaf17-ELN,

3—cDNA of ACT gene in bovine dermal fibroblast cells before transfection with gene therapy DNA vector VTvaf17-ELN,

4—cDNA of ACT gene in bovine dermal fibroblast cells after transfection with gene therapy DNA vector VTvaf17-ELN.

Bull/cow actin gene (ACT) listed in the GenBank database under number AH001130.2 was used as a reference gene.

### EMBODIMENT OF THE INVENTION

Gene therapy DNA vectors carrying the human therapeutic genes designed to increase the expression level of these therapeutic genes in human and animal tissues were constructed based on 3165 bp DNA vector VTvaf17. The method of production of each gene therapy DNA vector carrying the therapeutic genes is to clone the protein coding sequence of the therapeutic gene selected from the group of the following genes: human COL1A1 gene (encodes COL1A1 protein), human COL1A2 gene (encodes COL1A2 protein), human P4HA1 gene (encodes P4HA1 protein), human P4HA2 gene (encodes P4HA2 protein), human COL7A1 gene (encodes COL7A1 protein), human CLCA2 gene (encodes CLCA2 protein), human ELN gene (encodes ELN protein), and human PLOD1 gene (encodes PLOD1 protein) to the polylinker of gene therapy DNA vector VTvaf17. It is known that the ability of DNA vectors to penetrate into eukaryotic cells is due mainly to the vector size. DNA vectors with the smallest size have higher penetration capability. Thus, the absence of elements in the vector that bear no functional load, but at the same time increase the vector DNA size is preferred. These features of DNA vectors were taken into account during the production of gene therapy DNA vectors based on gene therapy DNA vector VTvaf17 carrying the therapeutic gene selected from the group of COL1A1, COL1A2, P4HA1, P4HA2, COL7A1, CLCA2, ELN, and PLOD1 genes with no large non-functional sequences and antibiotic resistance genes in the vector, which, in addition to technological advantages and safe use, had allowed for the significant reduction of size of the produced gene therapy DNA vector VTvaf17 carrying the therapeutic gene selected from the group of COL1A1, COL1A2, P4HA1, P4HA2, COL7A1, CLCA2, ELN, and PLOD1 genes. Thus, the ability of the obtained gene therapy DNA vector to penetrate into eukaryotic cells is due to its small length.

Each of the following gene therapy DNA vectors: VTvaf17-COL1A1, or VTvaf17-COL1A2, or VTvaf17-P4HA1, or VTvaf17-P4HA2, or VTvaf17-COL7A1, or VTvaf17-CLCA2, or VTvaf17-ELN, or VTvaf17-PLOD1 was produced as follows: the coding region of the COL1A1, or COL1A2, or P4HA1, or P4HA2, or COL7A1, or CLCA2, or ELN, or PLOD1 therapeutic gene was cloned to DNA vector VTvaf17, and gene therapy DNA vector VTvaf17-COL1A1, SEQ ID No. 1, or VTvaf17-COL1A2, SEQ No. 2, or VTvaf17-P4HA1, SEQ ID No. 3, or VTvaf17-P4HA2, SEQ ID No. 4, or VTvaf17-COL7A1, SEQ ID No. 5, or VTvaf17-CLCA2, SEQ ID No. 6, or VTvaf17-ELN, SEQ ID No. 7, or VTvaf17-PLOD1, SEQ ID No. 8, respectively, was obtained. The coding region of COL1A1 gene (4410 bp), or COL1A2 gene (4116 bp), or P4HA1 gene (1607 bp), or P4HA2 gene (1605 bp), or COL7A1 gene (8838 bp), or CLCA2 gene (2833 bp), or ELN gene (2068 bp), or PLOD1 gene (2185 bp) was produced by extracting total RNA from the biological normal human tissue sample. The reverse transcription reaction was used for the synthesis of the first chain cDNA of human COL1A1, COL1A2, P4HA1, P4HA2, COL7A1, CLCA2, ELN, and PLOD1 genes. Amplification was performed using oligonucleotides produced for this purpose by the chemical synthesis method. The amplification product was cleaved by specific restriction endonucleases taking into account the optimal procedure for further cloning, and cloning to the gene therapy DNA vector VTvaf17 was performed by BamHI, EcoRI, and HindIII restriction sites located in the VTvaf17 vector polylinker. The selection of restriction sites was carried out in such a way that the cloned fragment entered the open reading frame of expression cassette of the vector VTvaf17, while the protein coding sequence did not contain restriction sites for the selected endonucleases. Experts in this field realise that the methodological implementation of gene therapy DNA vector VTvaf17-COL1A1, or VTvaf17-COL1A2, or VTvaf17-P4HA1, or VTvaf17-P4HA2, or VTvaf17-COL7A1, or VTvaf17-CLCA2, or VTvaf17-ELN, or VTvaf17-PLOD1 production can vary within the framework of the selection of known methods of molecular gene cloning and these methods are included in the scope of this invention. For example, different oligonucleotide sequences can be used to amplify COL1A1, or COL1A2, or P4HA1, or P4HA2, or COL7A1, or CLCA2, or ELN, or PLOD1 gene, different restriction endonucleases or laboratory techniques, such as ligation independent cloning of genes.

Gene therapy DNA vector VTvaf17-COL1A1, or VTvaf17-COL1A2, or VTvaf17-P4HA1, or VTvaf17-P4HA2, or VTvaf17-COL7A1, or VTvaf17-CLCA2, or VTvaf17-ELN, or VTvaf17-PLOD1 has the nucleotide sequence SEQ ID No. 1, or SEQ ID No. 2, or SEQ ID No. 3, or SEQ ID No. 4, or SEQ ID No. 5, SEQ ID No. 6, or SEQ ID No. 7, or SEQ ID No. 8, respectively. At the same time, degeneracy of genetic code is known to the experts in this field and means that the scope of this invention also includes variants of nucleotide sequences differing by insertion, deletion, or replacement of nucleotides that do not result in a change in the polypeptide sequence encoded by the therapeutic gene, and/or do not result in a loss of functional activity of the regulatory elements of VTvaf17 vector. At the same time, genetic polymorphism is known to the experts in this field and means that the scope of this invention also includes variants of nucleotide sequences of genes from COL1A1, COL1A2, P4HA1, P4HA2, COL7A1, CLCA2, ELN, and PLOD1 genes that also encode different variants of the amino acid sequences of COL1A1, COL1A2, P4HA1, P4HA2, COL7A1, CLCA2, ELN, and PLOD1 proteins that do not differ from those listed in their functional activity under physiological conditions.

The ability to penetrate into eukaryotic cells and express functional activity, i.e. the ability to express the therapeutic gene of the obtained gene therapy DNA vector VTvaf17-COL1A1, or VTvaf17-COL1A2, or VTvaf17-P4HA1, or VTvaf17-P4HA2, or VTvaf17-COL7A1, or VTvaf17-CLCA2, or VTvaf17-ELN, or VTvaf17-PLOD1 is confirmed by injecting the obtained vector into eukaryotic cells and subsequent analysis of the expression of specific mRNA and/or protein product of the therapeutic gene. The presence of specific mRNA in cells into which the gene therapy DNA vector VTvaf17-COL 1 Al , or VTvaf17-COL1A2, or VTvaf17-P4HA1, or VTvaf17-P4HA2, or VTvaf17-COL7A1, or VTvaf17-CLCA2, or VTvaf17-ELN, or VTvaf17-PLOD1 was injected shows the ability of the obtained vector to both penetrate into eukaryotic cells and express mRNA of the therapeutic gene. Furthermore, it is known to the experts in this field that the presence of mRNA gene is a mandatory condition, but not an evidence of the translation of protein encoded by the therapeutic gene. Therefore, in order to confirm properties of the gene therapy DNA vector VTvaf17-COL1A1, or VTvaf17-COL 1 A2, or VTvaf17-P4HA1, or VTvaf17-P4HA2, or VTvaf17-COL7A1, or VTvaf17-CLCA2, or VTvaf17-ELN, or VTvaf17-PLOD1 to express the therapeutic gene at the protein level in eukaryotic cells into which the gene therapy DNA vector was injected, analysis of the concentration of proteins encoded by the therapeutic genes was carried out using immunological methods. The presence of COL1A1, or COL1A2, or P4HA1, or P4HA2, or COL7A1, or CLCA2, or ELN, or PLOD1 protein confirms the efficiency of expression of therapeutic genes in eukaryotic cells and the possibility of increasing the protein concentration using the gene therapy DNA vector based on gene therapy DNA vector VTvaf17 carrying the therapeutic gene selected from the group of COL1A1, COL1A2, P4HA1, P4HA2, COL7A1, CLCA2, ELN, and PLOD1 genes.

Thus, in order to confirm the expression efficiency of gene therapy DNA vector VTvaf17-COL1A1 carrying the therapeutic gene, namely the OL1A1 gene, gene therapy DNA vector VTvaf17-COL1A2 carrying the therapeutic gene, namely the COL1A2 gene, gene therapy DNA vector VTvaf17-P4HA1 carrying the therapeutic gene, namely the P4HA1 gene, gene therapy DNA vector VTvaf17-P4HA2 carrying the therapeutic gene, namely the P4HA2 gene, gene therapy DNA vector VTvaf17-COL7A1 carrying the therapeutic gene, namely the COL7A1 gene, gene therapy DNA vector VTvaf17-CLCA2 carrying the therapeutic gene, namely the CLCA2 gene, gene therapy DNA vector VTvaf17-ELN carrying the therapeutic gene, namely the ELN gene, gene therapy DNA vector VTvaf17-PLOD1 carrying the therapeutic gene, namely the PLOD1 gene, the following methods were used:

A) real-time PCR, i.e. change in mRNA accumulation of therapeutic genes in human and animal cell lysate after transfection of different human and animal cell lines with gene therapy DNA vectors,

B) Enzyme-linked immunosorbent assay, i.e. change in the quantitative level of therapeutic proteins in the human cell lysate after transfection of different human cell lines with gene therapy DNA vectors,

C) Enzyme-linked immunosorbent assay, i.e. change in the quantitative level of therapeutic proteins in the supernatant of human and animals tissue biopsy specimens after the injection of gene therapy DNA vectors into these tissues,

D) Enzyme-linked immunosorbent assay, i.e. change in the quantitative level of therapeutic proteins in the supernatant of human tissue biopsies after the injection of these tissues with autologous cells of this human transfected with gene therapy DNA vectors.

In order to confirm the practicability of use of the constructed gene therapy DNA vector VTvaf17-COL1A1 carrying the therapeutic gene, namely the COL1A1 gene, gene therapy DNA vector VTvaf17-COL1A2 carrying the therapeutic gene, namely the COL1A2 gene, gene therapy DNA vector VTvaf17-P4HA1 carrying the therapeutic gene, namely the P4HA1 gene, gene therapy DNA vector VTvaf17-P4HA2 carrying the therapeutic gene, namely the NP4HA2 gene, gene therapy DNA vector VTvaf17-COL7A1 carrying the therapeutic gene, namely the COL7A1 gene, gene therapy DNA vector VTvaf17-CLCA2 carrying the therapeutic gene, namely the CLCA2 gene, gene therapy DNA vector VTvaf17-ELN carrying the therapeutic gene, namely the ELN gene, gene therapy DNA vector VTvaf17-PLOD1 carrying the therapeutic gene, namely the PLOD1 gene, the following was performed:

A) transfection of different human and animal cell lines with gene therapy DNA vectors,

B) injection of gene therapy DNA vectors into different human and animal tissues,

C) injection of a mixture of gene therapy DNA vectors into human and animal tissues,

D) injection of autologous cells transfected with gene therapy DNA vectors into human tissues.

These methods of use lack potential risks for gene therapy of humans and animals due to the absence of regulatory elements in the gene therapy DNA vector that constitute the nucleotide sequences of viral genomes and absence of antibiotic resistance genes in the gene therapy DNA vector as confirmed by the lack of regions homologous to the viral genomes and antibiotic resistance genes in the nucleotide sequences of gene therapy DNA vector VTvaf17-COL1A1, or gene therapy DNA vector VTvaf17-COL1A2, or gene therapy DNA vector VTvaf17-P4HA1, or gene therapy DNA vector VTvaf17-P4HA2, or gene therapy DNA vector VTvaf17-COL7A1, or gene therapy DNA vector VTvaf17-CLCA2, or gene therapy DNA vector VTvaf17-ELN, or gene therapy DNA vector VTvaf17-PLOD1 (SEQ ID No. 1, or SEQ ID No. 2, or SEQ ID No. 3, or SEQ ID No. 4, or SEQ ID No. 5, or SEQ ID No. 6, or SEQ ID No. 7, or SEQ ID No. 8, respectively).

It is known to the experts in this field that antibiotic resistance genes in the gene therapy DNA vectors are used to obtain these vectors in preparative quantities by increasing bacterial biomass in a nutrient medium containing a selective antibiotic. Within the framework of this invention, in order to ensure the safe use of gene therapy DNA vector VTvaf17 carrying COL1A1, or COL1A2, or P4HA1, or P4HA2, or COL7A1, or CLCA2, or ELN, or PLOD1 therapeutic genes, the use of selective nutrient media containing an antibiotic is not possible. A method for obtaining strains for production of these gene therapy vectors based on Escherichia coli strain SCS110-AF is proposed as a technological solution for obtaining the gene therapy DNA vector VTvaf17 carrying a therapeutic gene selected from the group of COL1A1, COL1A2, P4HA1, P4HA2, COL7A1, CLCA2, ELN, and PLOD1 genes in order to scale up the production of gene therapy vectors to an industrial scale. The method of Escherichia coli strain SCS110-AF/VTvaf17-COL1A1, or Escherichia coli strain SCS110-AFNTvaf17-COL1A2, or Escherichia coli strain SCS110-AF/VTvaf17-P4HA1, or Escherichia coli strain SCS110-AFNTvaf17-P4HA2, or Escherichia coli strain SCS110-AFNTvaf17-COL7A1, or Escherichia coli strain SCS110-AF/VTvaf17-CLCA2, or Escherichia coli SCS110-AF/VTvaf17-ELN, or Escherichia coli strain SCS110-AFNTvaf17-PLOD1 production involves production of competent cells of Escherichia coli strain SCS110-AF with the injection of gene therapy DNA vector VTvaf17-COL1A1, or DNA vector VTvaf17-COL1A2, or DNA vector VTvaf17-P4HA1, or DNA vector VTvaf17-P4HA2, or DNA vector VTvaf17-COL7A1, or DNA vector VTvaf17-CLCA2, or DNA vector VTvaf17-ELN, or DNA vector VTvaf17-PLOD1 into these cells, respectively, using transformation (electroporation) methods widely known to experts in this field. The obtained Escherichia coli strain SCS110-AFNTvaf17-COL1A1, or Escherichia coli strain SCS110-AF/VTvaf17-COL1A2, or Escherichia coli strain SCS110-AFNTvaf17-P4HA1, or Escherichia coli strain SCS110-AFNTvaf17-P4HA2, or Escherichia coli strain SCS110-AF/VTvaf1 7-COL7A1, or Escherichia coli strain SCS110-AFNTvaf17-CLCA2, or Escherichia coli SCS110-AFNTvaf17-ELN, or Escherichia coli strain SCS110-AF/VTvaf17-PLOD1 is used to produce the gene therapy DNA vector VTvaf17-COL1A1, or VTvaf17-COL1A2, or VTvaf17-P4HA1, or VTvaf17-P4HA2, or VTvaf17-COL7A1, or VTvaf17-CLCA2, or VTvaf17-ELN, or VTvaf17-PLOD1, respectively, allowing for the use of antibiotic-free media.

In order to confirm the construction of Escherichia coli strain SCS110-AFNTvaf17-COL1A1, or Escherichia coli strain SCS110-AF/VTvaf17-COL1A2, or Escherichia coli strain SCS110-AFNTvaf17-P4HA1, or Escherichia coli strain SCS110-AFNTvaf17-P4HA2, or Escherichia coli strain SCS110-AF/VTvaf1 7-COL7A1, or Escherichia coli strain SCS110-AFNTvaf17-CLCA2, or Escherichia coli SCS110-AFNTvaf17-ELN, or Escherichia coli strain SCS110-AF/VTvaf17-PLOD1, transformation, selection, and subsequent biomass growth with extraction of plasmid DNA were performed.

To confirm the producibility, constructability and scale up of the production of gene therapy DNA vector VTvaf17-COL1A1 carrying the therapeutic gene, namely the COL1A1 gene, or gene therapy DNA vector VTvaf17-COL1A2 carrying the therapeutic gene, namely the COL1A2 gene, or gene therapy DNA vector VTvaf17-P4HA1 carrying the therapeutic gene, namely the P4HA1 gene, or gene therapy DNA vector VTvaf17-P4HA2 carrying the therapeutic gene, namely the NP4HA2 gene, or gene therapy DNA vector VTvaf17-COL7A1 carrying the therapeutic gene, namely the COL7A1 gene, or gene therapy DNA vector VTvaf17-CLCA2 carrying the therapeutic gene, namely the CLCA2 gene, or gene therapy DNA vector VTvaf17-ELN carrying the therapeutic gene, namely the ELN gene, or gene therapy DNA vector VTvaf17-PLOD1 carrying the therapeutic gene, namely the PLOD1 gene, to an industrial scale, the fermentation on an industrial scale of Escherichia coli strain SCS110-AF/VTvaf1 7-COL 1A1, or Escherichia coli strain SCS 110-AF/VTvaf17-COL 1A2, or Escherichia coli strain SCS110-AFNTvaf17-P4HA1, or Escherichia coli strain SCS110-AF/VTvaf1 7-P4HA2, or Escherichia coli strain SCS110-AFNTvaf17-COL7A1, or Escherichia coli strain SCS110-AFNTvaf17-CLCA2, or Escherichia coli SCS110-AF/VTvaf17-ELN, or Escherichia coli strain SCS110-AF/VTvaf17-PLOD1 each containing gene therapy DNA vector VTvaf17 carrying the therapeutic gene, namely COL1A1, or COL1A2, or P4HA1, or P4HA2, or COL7A1, or CLCA2, or ELN, or PLOD1 gene was performed.

The method of scaling the production of bacterial mass to an industrial scale for the isolation of gene therapy DNA vector VTvaf17 carrying the therapeutic gene selected from the group of COL1A1, COL1A2, P4HA1, P4HA2, COL7A1, CLCA2, ELN, and PLOD1 genes involves incubation of the seed culture of Escherichia coli strain SCS110-AFNTvaf17-COL1A1, or Escherichia coli strain SCS 110-AF/VTvaf17-COL1A2, or Escherichia coli strain SCS110-AFNTvaf17-P4HA1, or Escherichia coli strain SCS110-AFNTvaf17-P4HA2, or Escherichia coli strain SCS110-AF/VTvaf1 7-COL7A1, or Escherichia coli strain SCS110-AFNTvaf17-CLCA2, or Escherichia coli SCS 110-AF/VTvaf17-ELN, or Escherichia coli strain SCS110-AFNTvaf17-PLOD1 in the antibiotic-free nutrient medium that provides suitable biomass accumulation dynamics. Upon reaching a sufficient amount of biomass in the logarithmic phase, the bacterial culture is transferred to an industrial fermenter and then grown to a stationary phase, then the fraction containing the therapeutic DNA product, i.e. the gene therapy DNA vector VTvaf17-COL1A1, or DNA vector VTvaf17-COL1A2, or DNA vector VTvaf17-P4HA1, or DNA vector VTvaf17-P4HA2, or DNA vector VTvaf17-COL7A1, or DNA vector VTvaf17-CLCA2, or DNA vector VTvaf17-ELN, or DNA vector VTvaf17-PLOD1 is extracted, multi-stage filtered, and purified by chromatographic methods. It is known to the experts in this field that culture conditions of strains, composition of nutrient media (except for antibiotic-free), equipment used, and DNA purification methods may vary within the framework of standard operating procedures depending on the particular production line, but known approaches to scaling, industrial production, and purification of DNA vectors using Escherichia coli strain SCS110-AFNTvaf17-COL1A1, or Escherichia coli strain SCS110-AF/VTvaf17-COL1A2, or Escherichia coli strain SCS110-AFNTvaf17-P4HA1, or Escherichia coli strain SCS110-AFNTvaf17-P4HA2, or Escherichia coli strain SCS110-AF/VTvaf17-COL7A1, or Escherichia coli strain SCS110-AFNTvaf17-CLCA2, or Escherichia coli SCS110-AFNTvaf17-ELN, or Escherichia coli strain SCS110-AFNTvaf17-PLOD1 fall within the scope of this invention.

The essence of the invention is explained in the following examples.

### EXAMPLE 1

Production of gene therapy DNA vector VTvaf17-COL1A1 carrying the therapeutic gene, namely the COL1A1 gene.

Gene therapy DNA vector VTvaf17-COL1A1 was constructed by cloning the coding region of COL1A1 gene (4410 bp) to a 3165 bp DNA vector VTvaf17 by NheI and HindIII restriction sites. The coding region of COL1A1 gene (4410 bp) was obtained by isolating total RNA from the biological human tissue sample followed by reverse transcription reaction using commercial kit Mint-2 (Evrogen, Russia) and PCR amplification using the following oligonucleotides:

Col1A1_F
CCAGCTAGCGTCTAGGGTCTAGACATGTTC,
Col1A1_R
TATAAGCTTCTACAGGAAGCAGACAGGGCCAAC

and commercially available kit Phusion® High-Fidelity DNA Polymerase (New England Biolabs, USA).

Gene therapy DNA vector VTvaf17 was constructed by consolidating six fragments of DNA derived from different sources:

(a) the origin of replication was produced by PCR amplification of a region of commercially available plasmid pBR322 with a point mutation,

(b) EF 1 a promoter region was produced by PCR amplification of a site of human genomic DNA,

(c) hGH-TA transcription terminator was produced by PCR amplification of a site of human genomic DNA,

(d) the RNA-OUT regulatory site of transposon Tn10 was synthesised from oligonucleotides,

(e) kanamycin resistance gene was produced by PCR amplification of a site of commercially available human plasmid pET-28,

(f) the polylinker was produced by annealing two synthetic oligonucleotides. PCR amplification was performed using the commercially available kit Phusion® High-Fidelity DNA Polymerase (New England Biolabs, USA) as per the manufacturer's instructions. The fragments have overlapping regions allowing for their consolidation with subsequent PCR amplification. Fragments (a) and (b) were consolidated using oligonucleotides Ori-F and EF1-R, and fragments (c), (d), and (e) were consolidated using oligonucleotides hGH-F and Kan-R. Afterwards, the produced fragments were consolidated by restriction with subsequent ligation by sites BamHI and NcoI. This resulted in a plasmid still devoid of the polylinker. To add it, the plasmid was cleaved by BamHI and EcoRI sites followed by ligation with fragment (f). Therefore, a 4182 bp vector was constructed carrying the kanamycin resistance gene flanked by SpeI restriction sites. Then this gene was cleaved by SpeI restriction sites and the remaining fragment was ligated to itself. This resulted in a 3165 bp gene therapy DNA vector VTvaf17 that is recombinant and allows for antibiotic-free selection.

The amplification product of the coding region of COL1A1 gene and DNA vector VTvaf17 was cleaved by NheI and HindIII restriction endonucleases (New England Biolabs, USA).

This resulted in a 7563 bp DNA vector VTvaf17-COL1A1 with the nucleotide sequence SEQ ID No. 1 and general structure shown in FIG. 1A.

### EXAMPLE 2

Production of gene therapy DNA vector VTvaf17-COL1A2 carrying the therapeutic gene, namely the COL1A2 gene.

Gene therapy DNA vector VTvaf17-COL1A2 was constructed by cloning the coding region of COL1A2 gene (4116 bp) to a 3165 bp DNA vector VTvaf17 by NheI and HindIII restriction sites. The coding region of COL1A2 gene (4116 bp) was obtained by isolating total RNA from the biological human tissue sample followed by reverse transcription reaction using commercial kit Mint-2 (Evrogen, Russia) and PCR amplification using the following oligonucleotides:

Col1A2_F
CCAGCTAGCGTCTAAGTGCTAGACATGCTC,
Col1A2_R
CGAAGCTTTTATTTGAAACAGACTGGGCCA

and commercially available kit Phusion® High-Fidelity DNA Polymerase (New England Biolabs, USA); amplification product and DNA vector VTvaf17 were cleaved by restriction endonucleases NheI and HindIII (New England Biolabs, USA).

This resulted in a 7269 bp DNA vector VTvaf17-COL1A2 with the nucleotide sequence SEQ ID No. 2 and general structure shown in FIG. 1B.

Gene therapy DNA vector VTvaf17 was constructed as described in Example 1.

### EXAMPLE 3

Production of gene therapy DNA vector VTvaf17-P4HA1 carrying the therapeutic gene, namely the human P4HA1 gene.

Gene therapy DNA vector VTvaf17-P4HA1 was constructed by cloning the coding region of P4HA1 gene (1607 bp) to a 3165 bp DNA vector VTvaf17 by BamHI and KpnI restriction sites. The coding region of P4HA1 gene (1607 bp) was obtained by isolating total RNA from the biological human tissue sample followed by reverse transcription reaction using commercial kit Mint-2 (Evrogen, Russia) and PCR amplification using the following oligonucleotides:

P4HA1_F
AGGATCCACCATGATCTGGTATATATTAATTATAGG,
P4HA1_R
TTCGGTACCTATTCCAATTCTGACAACGTACAAG

and commercially available kit Phusion® High-Fidelity DNA Polymerase (New England Biolabs, USA); amplification product and DNA vector VTvaf17 were cleaved by BamHI and KpnI restriction endonucleases (New England Biolabs, USA).

This resulted in a 4754 bp DNA vector VTvaf17-P4HA1 with the nucleotide sequence SEQ ID No. 3 and general structure shown in FIG. 1C.

Gene therapy DNA vector VTvaf17 was constructed as described in Example 1.

### EXAMPLE 4

Production of gene therapy DNA vector VTvaf17-P4HA2 carrying the therapeutic gene, namely the P4HA2 gene.

Gene therapy DNA vector VTvaf17-P4HA2 was constructed by cloning the coding region of P4HA2 gene (1605 bp) to a 3165 bp DNA vector VTvaf17 by BamHII and SalI restriction sites. The coding region of P4HA2 gene (1605 bp) was obtained by isolating total RNA from the biological human tissue sample followed by reverse transcription reaction using commercial kit Mint-2 (Evrogen) and PCR amplification using the following oligonucleotides:

P4HA2_F
AGGATCCACCATGAAACTCTGGGTGTCTGCA,
P4HA2_R
CTTGTCGACTTAGTCAACTTCTGTTGATCCACA

and commercially available kit Phusion® High-Fidelity DNA Polymerase (New England Biolabs, USA); amplification product and DNA vector VTvaf17 were cleaved by BamHII and SalI restriction endonucleases (New England Biolabs, USA).

This resulted in a 4704 bp DNA vector VTvaf17-P4HA2 with the nucleotide sequence SEQ ID No. 4 and general structure shown in FIG. 1D.

Gene therapy DNA vector VTvaf17 was constructed as described in Example 1.

### EXAMPLE 5

Production of gene therapy DNA vector VTvaf17-COL7A1 carrying the therapeutic gene, namely the COL7A1 gene.

Gene therapy DNA vector VTvaf17-COL7A1 was constructed by cloning the coding region of COL7A1 gene (8838 bp) to a 3165 bp DNA vector VTvaf17 by SalI and EcoRI restriction sites. The coding region of COL7A1 gene (8838 bp) was obtained by isolating total RNA from the biological human tissue sample followed by reverse transcription reaction using commercial kit Mint-2 (Evrogen, Russia) and PCR amplification using the following oligonucleotides:

COL7A1_F
ATCGTCGACCACCATGACGCTGCGGCTTCTGGT,
COL7A1_R
ATAGAATTCAGTCCTGGGCAGTACCTGTC

and commercially available kit Phusion® High-Fidelity DNA Polymerase (New England Biolabs, USA); amplification product and DNA vector VTvaf17 were cleaved by restriction endonucleases SalI and EcoRI (New England Biolabs, USA).

This resulted in a 11990 bp DNA vector VTvaf17-COL7A1 with the nucleotide sequence SEQ ID No. 5 and general structure shown in FIG. 1E.

Gene therapy DNA vector VTvaf17 was constructed as described in Example 1.

### EXAMPLE 6

Production of gene therapy DNA vector VTvaf17-CLCA2 carrying the therapeutic gene, namely the human CLCA2 gene.

Gene therapy DNA vector VTvaf17-CLCA2 was constructed by cloning the coding region of CLCA2 gene (2833 bp) to a 3165 bp DNA vector VTvaf17 by BamHI and EcoRI restriction sites. The coding region of CLCA2 gene (2833 bp) was obtained by isolating total RNA from the biological human tissue sample followed by reverse transcription reaction using commercial kit Mint-2 (Evrogen, Russia) and PCR amplification using the following oligonucleotides:

CLCA2_F
AGGATCCACCATGACCCAAAGGAGCATTGC,
CLCA2_R
ATAGAATTCATAATAATTTTGTTCCATTCTCTTTC

and commercially available kit Phusion® High-Fidelity DNA Polymerase (New England Biolabs, USA); amplification product and DNA vector VTvaf17 were cleaved by restriction endonucleases BamHI and EcoRI (New England Biolabs, USA).

This resulted in a 5974 bp DNA vector VTvaf17-CLCA2 with the nucleotide sequence SEQ ID No. 6 and general structure shown in FIG. 1F.

Gene therapy DNA vector VTvaf17 was constructed as described in Example 1.

### EXAMPLE 7

Production of gene therapy DNA vector VTvaf17-ELN carrying the therapeutic gene, namely the ELN gene.

Gene therapy DNA vector VTvaf17-ELN was constructed by cloning the coding region of ELN gene (2068 bp) to a 3165 bp DNA vector VTvaf17 by SalI and EcoRI restriction sites. The coding region of ELN gene (2068 bp) was obtained by isolating total RNA from the biological human tissue sample followed by reverse transcription reaction using commercial kit Mint-2 (Evrogen) and PCR amplification using the following oligonucleotides:

ELN_F
TTTGTCGACCACCATGGCGGGTCTGACGGCGG,
ELN_R
TTTTTGAATTCTCATTTTCTCTTCCGGCCACAAGCTT

and commercially available kit Phusion® High-Fidelity DNA Polymerase (New England Biolabs, USA); amplification product and DNA vector VTvaf17 were cleaved by restriction endonucleases SalI and EcoRI (New England Biolabs, USA).

This resulted in a 5257 bp DNA vector VTvaf17-ELN with the nucleotide sequence SEQ ID No. 7 and general structure shown in FIG. 1G.

Gene therapy DNA vector VTvaf17 was constructed as described in Example 1.

### EXAMPLE 8

Production of gene therapy DNA vector VTvaf17-PLOD1 carrying the therapeutic gene, namely the PLOD1 gene.

Gene therapy DNA vector VTvaf17-PLOD1 was constructed by cloning the coding region of PLOD1 gene (2185 bp) to a 3165 bp DNA vector VTvaf17 by BamHII and EcoRI restriction sites. The coding region of PLOD1 gene (2185 bp) was obtained by isolating total RNA from the biological human tissue sample followed by reverse transcription reaction using commercial kit Mint-2 (Evrogen) and PCR amplification using the following oligonucleotides:

PLOD1_F
GGATCCACCATGCGGCCCCTGCTGCTACT,
PLOD1_R
ATAGAATTCAGGGATCGACGAAGGAGACT

and commercially available kit Phusion® High-Fidelity DNA Polymerase (New England Biolabs, USA); amplification product and DNA vector VTvaf17 were cleaved by restriction endonucleases BamHII and EcoRI (New England Biolabs, USA).

This resulted in a 5326 bp DNA vector VTvaf17-PLOD1 with the nucleotide sequence SEQ ID No. 8 and general structure shown in FIG. 1H.

Gene therapy DNA vector VTvaf17 was constructed as described in Example 1.

### EXAMPLE 9

Proof of the ability of gene therapy DNA vector VTvaf17-COL1A1 carrying the therapeutic gene, namely COL1A1 gene, to penetrate eukaryotic cells and its functional activity at the level of therapeutic gene mRNA expression. This example also demonstrates practicability of use of gene therapy DNA vector carrying the therapeutic gene.

Changes in the mRNA accumulation of the COL1A1 therapeutic gene were assessed in HDFa primary human dermal fibroblast cell culture (ATCC PCS-201-01) 48 hours after its transfection with gene therapy DNA vector VTvaf17-COL1A1 carrying the human COL1A1 gene. The amount of mRNA was determined by the dynamics of accumulation of cDNA amplicons in the real-time PCR.

HDFa primary human dermal fibroblast cell culture was used for the assessment of changes in the therapeutic COL1A1 mRNA accumulation. HDFa cell culture was grown under standard conditions (37° C., 5% CO2) using the Fibroblast Growth Kit—Serum-Free (ATCC® PCS-201-040). The growth medium was replaced every 48 hours during the cultivation process.

To achieve 90% confluence, 24 hours before the transfection procedure, the cells were seeded into a 24-well plate in the quantity of 5×10⁴ cells per well. Transfection with gene therapy DNA vector VTvaf17-COL1A1 expressing the human COL1A1 gene was performed using Lipofectamine 3000 (ThermoFisher Scientific, USA) according to the manufacturer's recommendations. In test tube 1, 1 μL of DNA vector VTvaf17-COL1A1 solution (concentration 500 ng/μl) and 1 μl of reagent P3000 was added to 25 μl of medium Opti-MEM (Gibco, USA). The preparation was mixed by gentle shaking. In test tube 2, 1 μl of Lipofectamine 3000 solution was added to 25 μl of medium Opti-MEM (Gibco, USA). The preparation was mixed by gentle shaking. The contents from test tube 1 were added to the contents of test tube 2, and the mixture was incubated at room temperature for 5 minutes. The resulting solution was added dropwise to the cells in the volume of 40 μl.

HDFa cells transfected with the gene therapy DNA vector VTvaf17 devoid of the inserted therapeutic gene (cDNA of COL1A1 gene before and after transfection with gene therapy DNA vector VTvaf17 devoid of the inserted therapeutic gene is not shown in the figures) were used as a reference. Reference vector VTvaf17 for transfection was prepared as described above.

Total RNA from HDFa cells was extracted using Trizol Reagent (Invitrogen, USA) according to the manufacturer's recommendations. 1 ml of Trizol Reagent was added to the well with cells, homogenised and heated for 5 minutes at 65° C. The sample was centrifuged at 14,000 g for 10 minutes and heated again for 10 minutes at 65° C. Then, 200 μl of chloroform was added, and the mixture was gently stirred and centrifuged at 14,000 g for 10 minutes. Then the water phase was isolated and mixed with 1/10 of the volume of 3M sodium acetate, pH 5.2, and an equal volume of isopropyl alcohol. The sample was incubated at −20° C. for 10 minutes and then centrifuged at 14,000 g for 10 minutes. The precipitated RNA were rinsed in 1 ml of 70% ethyl alcohol, air-dried and dissolved in 10 μl of RNase-free water. The level of COL1A1 mRNA expression after transfection was determined by assessing the dynamics of the accumulation of cDNA amplicons by real-time PCR. For the production and amplification of cDNA specific for the human COL1A1 gene, the following COL1A1 SF and COL1A1 SR oligonucleotides were used:

Col1A1_SF
TGACCTCAAGATGTGCCACT,
Col1A1_SR
CAGACATGCCTCTTGTCCTTG

The length of amplification product is 195 bp.

Reverse transcription reaction and PCR amplification was performed using SYBR GreenQuantitect RT—PCR Kit (Qiagen, USA) for real-time PCR. The reaction was carried out in a volume of 20 μl, containing: 25 μl of QuantiTect SYBR Green RT-PCR Master Mix, 2.5 mM of magnesium chloride, 0.5 μM of each primer, and 5 μl of RNA. For the reaction, CFX96 amplifier (Bio-Rad, USA) was used under the following conditions: 1 cycle of reverse transcription at 42° C. for 30 minutes, denaturation at 98° C. for 15 minutes, followed by 40 cycles comprising denaturation at 94° C. for 15 s, annealing of primers at 60° C. for 30 s and elongation at 72° C. for 30 s. B2M (beta-2-microglobuline) gene listed in the GenBank database under number NM 004048.2 was used as a reference gene. Positive control included amplicons from PCR on matrices represented by plasmids in known concentrations containing cDNA sequences of COL1A1 and B2M genes. Negative control included deionised water. Real-time quantification of the dynamics of accumulation of cDNA amplicons of COL1A1 and B2M genes was conducted using the Bio-Rad CFX Manager 2.1 software (Bio-Rad, USA). Diagrams resulting from the assay are shown in FIG. 2.

FIG. 2 shows that the level of specific mRNA of human COL1A1 gene has grown massively as a result of transfection of HDFa primary human fibroblast cell culture with gene therapy DNA vector VTvaf17-COL1A1, which confirms the ability of the vector to penetrate eukaryotic cells and express the COL1A1 gene at the mRNA level. The presented results also confirm the practicability of use of gene therapy DNA vector VTvaf17-COL1A1 in order to increase the expression level of COL1A1 gene in eukaryotic cells.

### EXAMPLE 10

Proof of the ability of gene therapy DNA vector VTvaf17-COL1A2 carrying the therapeutic gene, namely COL1A2 gene, to penetrate eukaryotic cells and its functional activity at the level of therapeutic gene mRNA expression. This example also demonstrates practicability of use of gene therapy DNA vector carrying the therapeutic gene.

Changes in the mRNA accumulation of the COL1A2 therapeutic gene were assessed in HDFa primary human dermal fibroblast cell culture (ATCC PCS-201-01) 48 hours after its transfection with gene therapy DNA vector VTvaf17-COL1A2 carrying the human COL1A2 gene. The amount of mRNA was determined by the dynamics of accumulation of cDNA amplicons in the real-time PCR.

HDFa primary human dermal fibroblast cell culture was grown in Fibroblast Growth Kit—Serum-Free (ATCC® PCS-201-040) under standard conditions (37° C., 5% CO2). To achieve 90% confluence, 24 hours before the transfection procedure, the cells were seeded into a 24-well plate in the quantity of 5×10⁴ cells per well. Lipofectamine 3000 (ThermoFisher Scientific, USA) was used as a transfection reagent. The transfection with gene therapy DNA vector VTvaf17-COL1A2 expressing the human COL1A2 gene was performed according to the procedure described in Example 9. B2M (beta-2-microglobuline) gene listed in the GenBank database under number NM 004048.2 was used as a reference gene. HDFa cell culture transfected with the gene therapy DNA vector VTvaf17 devoid of the therapeutic gene (cDNA of COL1A2 gene before and after transfection with gene therapy DNA vector VTvaf17 devoid of the inserted therapeutic gene is not shown in the figures) was used as a reference. RNA isolation, reverse transcription reaction, and real-time PCR were performed as described in Example 9, except for oligonucleotides with sequences different from Example 9. For the amplification of cDNA specific for the human COL1A2 gene, the following COL1A2_SF and COL1A2_SR oligonucleotides were used:

COL1A2_SF
TGAACTTGTTGCTGAGGGCA,
COL1A2_SR
CCAGTTCTTGGCTGGGATGT

The length of amplification product is 195 bp.

Positive control included amplicons from PCR on matrices represented by plasmids in known concentrations containing cDNA sequences of COL1A2 and B2M genes. Negative control included deionised water. Real-time quantification of the PCR products, i.e. COL1A2 and B2M gene cDNAs obtained by amplification, was conducted using the Bio-Rad CFX Manager 2.1 software (Bio-Rad, USA). Diagrams resulting from the assay are shown in FIG. 3.

FIG. 3 shows that the level of specific mRNA of human COL1A2 gene has grown massively as a result of transfection of HDFa human fibroblast cell culture with gene therapy DNA vector VTvaf17-COL1A2, which confirms the ability of the vector to penetrate eukaryotic cells and express the COL1A2 gene at the mRNA level. The presented results also confirm the practicability of use of gene therapy DNA vector VTvaf17-COL1A2 in order to increase the expression level of COL1A2 gene in eukaryotic cells.

### EXAMPLE 11

Proof of the ability of gene therapy DNA vector VTvaf17-P4HA1 carrying the therapeutic gene, namely P4HA1 gene, to penetrate eukaryotic cells and its functional activity at the level of therapeutic gene mRNA expression. This example also demonstrates practicability of use of gene therapy DNA vector carrying the therapeutic gene.

Changes in the mRNA accumulation of the P4HA1 therapeutic gene were assessed in Hs27 human foreskin fibroblast cell line (ATCC CRL-1634) 48 hours after its transfection with gene therapy DNA vector VTvaf17-P4HA1 carrying the human P4HA1 gene. The amount of mRNA was determined by the dynamics of accumulation of cDNA amplicons in the real-time PCR.

Hs27 human foreskin fibroblast cell line was grown in Dulbecco's Modified Eagle's Medium (DMEM) (ATCC® 30-2020™) with the addition of 10% of bovine serum (ATCC® 30-2020™) under standard conditions (37° C., 5% CO2). To achieve 90% confluence, 24 hours before the transfection procedure, the cells were seeded into a 24-well plate in the quantity of 5×10⁴ cells per well. Lipofectamine 3000 (ThermoFisher Scientific, USA) was used as a transfection reagent. The transfection with gene therapy DNA vector VTvaf17-P4HA1 expressing the human P4HA1 gene was performed according to the procedure described in Example 9. B2M (beta-2-microglobuline) gene listed in the GenBank database under number NM 004048.2 was used as a reference gene. Hs27 cell line transfected with the gene therapy DNA vector VTvaf17 devoid of the therapeutic gene (cDNA of P4HA1 gene before and after transfection with gene therapy DNA vector VTvaf17 devoid of the inserted therapeutic gene is not shown in the figures) was used as a reference. RNA isolation, reverse transcription reaction, and real-time PCR were performed as described in Example 9, except for oligonucleotides with sequences different from Example 9. For the amplification of cDNA specific for the human P4HA1 gene, the following P4HA1_SF and P4HA1_SR oligonucleotides were used:

P4HA1_SF
AAAAGTGCCTGGCTCTCTGG,
P4HA1_SR
TGGCTCATCTTTCCGTGCAA

The length of amplification product is 171 bp.

Positive control included amplicons from PCR on matrices represented by plasmids in known concentrations containing cDNA sequences of P4HA1 and B2M genes. Negative control included deionised water. Real-time quantification of the PCR products, i.e. P4HA1 and B2M gene cDNAs obtained by amplification, was conducted using the Bio-Rad CFX Manager 2.1 software (Bio-Rad, USA). Diagrams resulting from the assay are shown in FIG. 4.

FIG. 4 shows that the level of specific mRNA of human P4HA1 gene has grown massively as a result of transfection of Hs27 human foreskin fibroblast cell line with gene therapy DNA vector VTvaf17-P4HA1, which confirms the ability of the vector to penetrate eukaryotic cells and express the P4HA1 gene at the mRNA level. The presented results also confirm the practicability of use of gene therapy DNA vector VTvaf17-P4HA1 in order to increase the expression level of P4HA1 gene in eukaryotic cells.

### EXAMPLE 12

Proof of the ability of gene therapy DNA vector VTvaf17-P4HA2 carrying the therapeutic gene, namely P4HA2 gene, to penetrate eukaryotic cells and its functional activity at the level of therapeutic gene mRNA expression. This example also demonstrates practicability of use of gene therapy DNA vector carrying the therapeutic gene.

Changes in the mRNA accumulation of the P4HA2 therapeutic gene were assessed in Hs27 human foreskin fibroblast cell line (ATCC CRL-1634) 48 hours after its transfection with gene therapy DNA vector VTvaf17-P4HA2 carrying the human P4HA2 gene. The amount of mRNA was determined by the dynamics of accumulation of cDNA amplicons in the real-time PCR.

Hs27 human foreskin fibroblast cell line was grown in Dulbecco's Modified Eagle's Medium (DMEM) (ATCC® 302020™) with the addition of 10% of bovine serum (ATCC® 30-2020™) under standard conditions (37° C., 5% CO2). To achieve 90% confluence, 24 hours before the transfection procedure, the cells were seeded into a 24-well plate in the quantity of 5×10⁴ cells per well. Lipofectamine 3000 (ThermoFisher Scientific, USA) was used as a transfection reagent. The transfection with gene therapy DNA vector VTvaf17-P4HA2 expressing the human P4HA2 gene was performed according to the procedure described in Example 9. Hs27 cell culture transfected with the gene therapy DNA vector VTvaf17 devoid of the therapeutic gene (cDNA of P4HA2 gene before and after transfection with gene therapy DNA vector VTvaf17 devoid of the inserted therapeutic gene is not shown in the figures) was used as a reference. RNA isolation, reverse transcription reaction, and real-time PCR were performed as described in Example 9, except for oligonucleotides with sequences different from Example 9. For the amplification of cDNA specific for the human P4HA2 gene, the following P4HA2 SF and P4HA2 SR oligonucleotides were used:

P4HA2_SF
AGGTACCACCATGGCAACAG,
P4HA2_SR
GTCTTGGGATCACGAACGGT

The length of amplification product is 179 bp.

Positive control included amplicons from PCR on matrices represented by plasmids in known concentrations containing cDNA sequences of P4HA2 and B2M genes. B2M (beta-2-microglobuline) gene listed in the GenBank database under number NM 004048.2 was used as a reference gene. Negative control included deionised water. Real-time quantification of the PCR products, i.e. P4HA2 and B2M gene cDNAs obtained by amplification, was conducted using the Bio-Rad CFX Manager 2.1 software (Bio-Rad, USA). Diagrams resulting from the assay are shown in FIG. 5.

FIG. 5 shows that the level of specific mRNA of human P4HA2 gene has grown massively as a result of transfection of Hs27 human foreskin fibroblast cell line with gene therapy DNA vector VTvaf17-P4HA2, which confirms the ability of the vector to penetrate eukaryotic cells and express the P4HA2 gene at the mRNA level. The presented results also confirm the practicability of use of gene therapy DNA vector VTvaf17-P4HA2 in order to increase the expression level of P4HA2 gene in eukaryotic cells.

### EXAMPLE 13

Proof of the ability of gene therapy DNA vector VTvaf17-COL7A1 carrying the therapeutic gene, namely COL7A1 gene, to penetrate eukaryotic cells and its functional activity at the level of therapeutic gene mRNA expression. This example also demonstrates practicability of use of gene therapy DNA vector carrying the therapeutic gene.

Changes in the mRNA accumulation of the COL7A1 therapeutic gene were assessed in HT 297.T human dermal fibroblast cell culture (ATCC® CRL-7782™) 48 hours after its transfection with gene therapy DNA vector VTvaf17-COL7A1 carrying the human COL7A1 gene. The amount of mRNA was determined by the dynamics of accumulation of cDNA amplicons in the real-time PCR.

HT 297.T human dermal fibroblast cell culture was grown in Dulbecco's Modified Eagle's Medium (DMEM) (ATCC® 30-2002™) with the addition of 10% of bovine serum (ATCC® 30-2020™) under standard conditions (37° C., 5% CO2). To achieve 90% confluence, 24 hours before the transfection procedure, the cells were seeded into a 24-well plate in the quantity of 5×10⁴ cells per well. Lipofectamine 3000 (ThermoFisher Scientific, USA) was used as a transfection reagent. The transfection with gene therapy DNA vector VTvaf17-COL7A1 expressing the human COL7A1 gene was performed according to the procedure described in Example 9. B2M (beta-2-microglobuline) gene listed in the GenBank database under number NM 004048.2 was used as a reference gene. HT 297.T cell culture transfected with the gene therapy DNA vector VTvaf17 devoid of the therapeutic gene (cDNA of COL7A1 gene before and after transfection with gene therapy DNA vector VTvaf17 devoid of the inserted therapeutic gene is not shown in the figures) was used as a reference. RNA isolation, reverse transcription reaction, and real-time PCR were performed as described in Example 9, except for oligonucleotides with sequences different from Example 9. For the amplification of cDNA specific for the human COL7A1 gene, the following COL7A1 SF and COL7A1 SR oligonucleotides were used:

COL7A1_SF
CAAAGGAGAGATGGGGGAGC,
COL7A1_SR
ATCATTTCCACTGGGGCCTG

The length of amplification product is 184 bp.

Positive control included amplicons from PCR on matrices represented by plasmids in known concentrations containing cDNA sequences of COL7A1 and B2M genes. Negative control included deionised water. Real-time quantification of the PCR products, i.e. COL7A1 and B2M gene cDNAs obtained by amplification, was conducted using the Bio-Rad CFX Manager 2.1 software (Bio-Rad, USA). Diagrams resulting from the assay are shown in FIG. 6.

FIG. 6 shows that the level of specific mRNA of human COL7A1 gene has grown massively as a result of transfection of HT 297.T human dermal fibroblast cell culture with gene therapy DNA vector VTvaf17-COL7A1, which confirms the ability of the vector to penetrate eukaryotic cells and express the COL7A1 gene at the mRNA level. The presented results also confirm the practicability of use of gene therapy DNA vector VTvaf17-COL7A1 in order to increase the expression level of the COL7A1 gene in eukaryotic cells.

### EXAMPLE 14

Proof of the ability of gene therapy DNA vector VTvaf17-CLCA2 carrying the therapeutic gene, namely CLCA2 gene, to penetrate eukaryotic cells and its functional activity at the level of therapeutic gene mRNA expression. This example also demonstrates practicability of use of gene therapy DNA vector carrying the therapeutic gene.

Changes in the mRNA accumulation of the CLCA2 therapeutic gene were assessed in HT 297.T human dermal fibroblast cell culture (ATCC® CRL-7782™) 48 hours after its transfection with gene therapy DNA vector VTvaf17-CLCA2 carrying the human CLCA2 gene. The amount of mRNA was determined by the dynamics of accumulation of cDNA amplicons in the real-time PCR.

HT 297.T human dermal fibroblast cell culture was grown in Dulbecco's Modified Eagle's Medium (DMEM) (ATCC® 30-2002™) with the addition of 10% of bovine serum (ATCC® 30-2020™) under standard conditions (37° C., 5% CO2). To achieve 90% confluence, 24 hours before the transfection procedure, the cells were seeded into a 24-well plate in the quantity of 5×10⁴ cells per well. Lipofectamine 3000 (ThermoFisher Scientific, USA) was used as a transfection reagent. The transfection with gene therapy DNA vector VTvaf17-CLCA2 expressing the human CLCA2 gene was performed according to the procedure described in Example 9. B2M (beta-2-microglobuline) gene listed in the GenBank database under number NM 004048.2 was used as a reference gene. HT 297.T cell line transfected with the gene therapy DNA vector VTvaf17 devoid of the therapeutic gene (cDNA of CLCA2 gene before and after transfection with gene therapy DNA vector VTvaf17 devoid of the inserted therapeutic gene is not shown in the figures) was used as a reference. RNA isolation, reverse transcription reaction, and real-time PCR were performed as described in Example 9, except for oligonucleotides with sequences different from Example 9. For the amplification of cDNA specific for the human CLCA2 gene, the following CLCA2 SF and CLCA2 SR oligonucleotides were used:

CLCA2_SF
GGAGGCTCCTTTTCAGTGCT,
CLCA2_SR
GTAGCCTGGCCCTGATCAAA

The length of amplification product is 155 bp.

Positive control included amplicons from PCR on matrices represented by plasmids in known concentrations containing cDNA sequences of CLCA2 and B2M genes. Negative control included deionised water. Real-time quantification of the PCR products, i.e. CLCA2 and B2M gene cDNAs obtained by amplification, was conducted using the Bio-Rad CFX Manager 2.1 software (Bio-Rad, USA). Diagrams resulting from the assay are shown in FIG. 7.

FIG. 7 shows that the level of specific mRNA of human CLCA2 gene has grown massively as a result of transfection of HT 297.T human dermal fibroblast cell culture with gene therapy DNA vector VTvaf17-CLCA2, which confirms the ability of the vector to penetrate eukaryotic cells and express the CLCA2 gene at the mRNA level. The presented results also confirm the practicability of use of gene therapy DNA vector VTvaf17-CLCA2 in order to increase the expression level of CLCA2 gene in eukaryotic cells.

### EXAMPLE 15

Proof of the ability of gene therapy DNA vector VTvaf17-ELN carrying the therapeutic gene, namely ELN gene, to penetrate eukaryotic cells and its functional activity at the level of therapeutic gene mRNA expression. This example also demonstrates practicability of use of gene therapy DNA vector carrying the therapeutic gene.

Changes in the mRNA accumulation of the ELN therapeutic gene were assessed in HEKa human epidermal keratinocyte cell culture (ATCC PCS-200-011) 48 hours after its transfection with gene therapy DNA vector VTvaf17-ELN carrying the human ELN gene. The amount of mRNA was determined by the dynamics of accumulation of cDNA amplicons in the real-time PCR.

HEKa human epidermal keratinocyte cell culture was grown in Keratinocyte Growth Kit (ATCC® PCS-200-0400™) under standard conditions (37° C., 5% CO2). To achieve 90% confluence, 24 hours before the transfection procedure, the cells were seeded into a 24-well plate in the quantity of 5×10⁴ cells per well. Lipofectamine 3000 (ThermoFisher Scientific, USA) was used as a transfection reagent. The transfection of the cells with gene therapy DNA vector VTvaf17-ELN expressing the human ELN gene was performed according to the procedure described in Example 9. HEKa cell culture transfected with the gene therapy DNA vector VTvaf17 devoid of the therapeutic gene (cDNA of ELN gene before and after transfection with gene therapy DNA vector VTvaf17 devoid of the inserted therapeutic gene is not shown in the figures) was used as a reference. RNA isolation, reverse transcription reaction, and real-time PCR were performed as described in Example 9, except for oligonucleotides with sequences different from Example 9. For the amplification of cDNA specific for the human ELN gene, the following ELN SF and ELN SR oligonucleotides were used:

ELN_SF
CCAGTTTGGCCTAGTGGGAG,
ELN_SR
ATGGGAGACAATCCGAAGCC

The length of amplification product is 159 bp.

Positive control included amplicons from PCR on matrices represented by plasmids in known concentrations containing cDNA sequences of ELN and B2M genes. B2M (beta-2-microglobuline) gene listed in the GenBank database under number NM 004048.2 was used as a reference gene. Negative control included deionised water. Real-time quantification of the PCR products, i.e. ELN and B2M gene cDNAs obtained by amplification, was conducted using the Bio-Rad CFX Manager 2.1 software (Bio-Rad, USA). Diagrams resulting from the assay are shown in FIG. 8.

FIG. 8 shows that the level of specific mRNA of human ELN gene has grown massively as a result of transfection of HEKa human epidermal keratinocyte cell culture with gene therapy DNA vector VTvaf17-ELN, which confirms the ability of the vector to penetrate eukaryotic cells and express the ELN gene at the mRNA level. The presented results also confirm the practicability of use of gene therapy DNA vector VTvaf17-ELN in order to increase the expression level of ELN gene in eukaryotic cells.

### EXAMPLE 16

Proof of the ability of gene therapy DNA vector VTvaf17-PLOD1 carrying the therapeutic gene, namely PLOD1 gene, to penetrate eukaryotic cells and its functional activity at the level of therapeutic gene mRNA expression. This example also demonstrates practicability of use of gene therapy DNA vector carrying the therapeutic gene.

Changes in the mRNA accumulation of the PLOD1 therapeutic gene were assessed in HEMa epidermal melanocyte cell culture (ATCC® PCS-200-013™) 48 hours after its transfection with gene therapy DNA vector VTvaf17-PLOD1 carrying the human PLOD1 gene. The amount of mRNA was determined by the dynamics of accumulation of cDNA amplicons in the real-time PCR.

HEMa primary human epidermal melanocyte cell culture was grown in Dermal Cell Basal Medium (ATCC® PCS-200-030™) with the addition of Adult Melanocyte Growth Kit (ATCC® PCS-200-042™) under standard conditions (37° C., 5% CO2). To achieve 90% confluence, 24 hours before the transfection procedure, the cells were seeded into a 24-well plate in the quantity of 5×10⁴ cells per well. Lipofectamine 3000 (ThermoFisher Scientific, USA) was used as a transfection reagent. The transfection with gene therapy DNA vector VTvaf17-PLOD1 expressing the human PLOD1 gene was performed according to the procedure described in Example 9. HEMa cell culture transfected with the gene therapy DNA vector VTvaf17 devoid of the therapeutic gene (cDNA of PLOD1 gene before and after transfection with gene therapy DNA vector VTvaf17 devoid of the inserted therapeutic gene is not shown in the figures) was used as a reference. RNA isolation, reverse transcription reaction, and real-time PCR were performed as described in Example 9, except for oligonucleotides with sequences different from Example 9. For the amplification of cDNA specific for the human PLOD1 gene, the following PLOD1_SF and PLOD1_SR oligonucleotides were used:

PLOD1_SF
GCCTCCACCTTCACCATCAA,
PLOD1_SR
AAGGAGACTGCGATGTAGCG

The length of amplification product is 197 bp.

Positive control included amplicons from PCR on matrices represented by plasmids in known concentrations containing cDNA sequences of PLOD1 and B2M genes. B2M (beta-2-microglobuline) gene listed in the GenBank database under number NM 004048.2 was used as a reference gene. Negative control included deionised water. Real-time quantification of the PCR products, i.e. PLOD1 and B2M gene cDNAs obtained by amplification, was conducted using the Bio-Rad CFX Manager 2.1 software (Bio-Rad, USA). Diagrams resulting from the assay are shown in FIG. 9.

FIG. 9 shows that the level of specific mRNA of human PLOD1 gene has grown massively as a result of transfection of HEMa epidermal melanocyte cell culture with gene therapy DNA vector VTvaf17-PLOD1, which confirms the ability of the vector to penetrate eukaryotic cells and express the PLOD1 gene at the mRNA level. The presented results also confirm the practicability of use of gene therapy DNA vector VTvaf17-PLOD1 in order to increase the expression level of PLOD1 gene in eukaryotic cells.

### EXAMPLE 17

Proof of the efficiency and practicability of use of gene therapy DNA vector VTvaf17-COL1A1 carrying the COL1A1 gene in order to increase the expression of COL1A1 protein in mammalian cells.

The change in the COL1A1 protein concentration in the lysate of HDFa human dermal fibroblast cells (ATCC PCS-201-01) was assessed after transfection of these cells with DNA vector VTvaf17-COL1A1 carrying the human COL1A1 gene.

HDFa human dermal fibroblast cell culture was grown as described in Example 9.

To achieve 90% confluence, 24 hours before the transfection procedure, the cells were seeded into a 24-well plate in the quantity of 5×10⁴ cells per well. The 6th generation SuperFect Transfection Reagent (Qiagen, Germany) was used for transfection. The aqueous dendrimer solution without DNA vector (A) and DNA vector VTvaf17 devoid of cDNA of COL1A1 gene (B) were used as a reference, and DNA vector VTvaf17-COL1A1 carrying the human COL1A1 gene (C) was used as the transfected agent. The DNA-dendrimer complex was prepared according to the manufacturer's procedure (QIAGEN, SuperFect Transfection Reagent Handbook, 2002) with some modifications. For cell transfection in one well of a 24-well plate, the culture medium was added to 1 μg of DNA vector dissolved in TE buffer to a final volume of 60 μl, then 5 l of SuperFect Transfection Reagent was added and gently mixed by pipetting five times. The complex was incubated at room temperature for 10-15 minutes. Then the culture medium was taken from the wells, the wells were rinsed with 1 ml of PBS buffer. 350 μl of medium containing 10 μm/ml of gentamicin was added to the resulting complex, mixed gently, and added to the cells. The cells were incubated with the complexes for 2-3 hours at 37° C. in the presence of 5% CO2.

The medium was then removed carefully, and the live cell array was rinsed with 1 ml of PBS buffer. Then, medium containing 10 μg/ml of gentamicin was added and incubated for 24-48 hours at 37° C. in the presence of 5% CO2.

After transfection, cells were rinsed three times with PBS, and then 1 ml of PBS was added to the cells and the cells were subjected to freezing/thawing three times. Then the suspension was centrifuged for 15 minutes at 15,000 rpm, and supernatant was collected and used for the quantification and assay of the therapeutic protein.

The COL1A1 protein was assayed by enzyme-linked immunosorbent assay (ELISA) using the Human COL1A1/Collagen I Alpha 1 ELISA Kit (Sandwich ELISA) (LifeSpan BioSciences, LS-F22003-1) according to the manufacturer's method with optical density detection using ChemWell Automated EIA and Chemistry Analyser (Awareness Technology Inc., USA).

To measure the numerical value of concentration, the calibration curve constructed using the reference samples from the kit with known concentrations of COL1A1 protein was used. The sensitivity was at least 188 pg/ml, measurement range—from 313 pg/ml to 20000 pg/ml. R-3.0.2 was used for the statistical treatment of the results and data visualization (https://www.r-project.org/). Drawings resulting from the assay are shown in FIG. 10.

FIG. 10 shows that the transfection of HDFa human dermal fibroblast cells with gene therapy DNA vector VTvaf17-COL1A1 results in increased COL1A1 protein concentration compared to reference samples, which confirms the ability of the vector to penetrate eukaryotic cells and express the COL1A1 gene at the protein level. The presented results also confirm the practicability of use of gene therapy DNA vector VTvaf17-COL1A1 in order to increase the expression level of the COL1A1 gene in eukaryotic cells.

### EXAMPLE 18

Proof of the efficiency and practicability of use of gene therapy DNA vector VTvaf17-COL1A2 carrying the COL1A2 gene in order to increase the expression of COL1A2 protein in mammalian cells.

The change in the COL1A2 protein concentration in the cell lysate of HDFa human dermal fibroblast cells (ATCC PCS-201-01) was assessed after transfection of these cells with DNA vector VTvaf17-COL1A2 carrying the human COL1A2 gene. Cells were grown as described in Example 10.

The 6th generation SuperFect Transfection Reagent (Qiagen, Germany) was used for transfection. The aqueous dendrimer solution without DNA vector (A) and DNA vector VTvaf17 devoid of cDNA of COL1A2 gene (B) were used as a reference, and DNA vector VTvaf17-COL1A2 carrying the human COL1A2 gene (C) was used as the transfected agent. Preparation of the DNA dendrimer complex and transfection of HDFa cells were performed according to the procedure described in Example 17.

After transfection, 0.1 ml of 1N HCl were added to 0.5 m of the culture broth, mixed thoroughly, and incubated for 10 minutes at room temperature. Then, the mixture was neutralised by adding 0.1 ml of 1.2M NaOH/0.5M HEPES (pH 7-7.6) and stirred thoroughly. Supernatant was collected and used to assay the therapeutic protein. The COL1A2 protein was assayed by enzyme-linked immunosorbent assay (ELISA) using the Human COL1A2/Collagen I Alpha 2 ELISA Kit (Sandwich ELISA) (LifeSpan Biosciences, LS-F26740) according to the manufacturer's method with optical density detection using ChemWell Automated EIA and Chemistry Analyser (Awareness Technology Inc., USA).

To measure the numerical value of concentration, the calibration curve constructed using the reference samples from the kit with known concentrations of COL1A2 protein was used. The sensitivity was at least 100 pg/ml, measurement range—from 500 pg/ml to 10000 pg/ml. R-3.0.2 was used for the statistical treatment of the results and data visualization (https://www.r-project.org/). Drawings resulting from the assay are shown in FIG. 11.

FIG. 11 shows that the transfection of HDFa primary human dermal fibroblast cell culture with gene therapy DNA vector VTvaf17-COL1A2 results in increased COL1A2 protein concentration compared to reference samples, which confirms the ability of the vector to penetrate eukaryotic cells and express the COL1A2 gene at the protein level. The presented results also confirm the practicability of use of gene therapy DNA vector VTvaf17-COL1A2 in order to increase the expression level of COL1A2 in eukaryotic cells.

### EXAMPLE 19

Proof of the efficiency and practicability of use of gene therapy DNA vector VTvaf17-P4HA1 carrying the P4HA1 gene in order to increase the expression of P4HA1 protein in mammalian cells.

Changes in the P4HA1 protein concentration in the lysate of Hs27 human foreskin fibroblast cell line (ATCC CRL-1634) were assessed after transfection of these cells with gene therapy DNA vector VTvaf17-P4HA1 carrying the human P4HA1 gene. Cells were cultured as described in Example 11.

The 6th generation SuperFect Transfection Reagent (Qiagen, Germany) was used for transfection. The aqueous dendrimer solution without DNA vector (A) and DNA vector VTvaf17 devoid of cDNA of P4HA1 gene (B) were used as a reference, and DNA vector VTvaf17-P4HA1 carrying the human P4HA1 gene (C) was used as the transfected agent. Preparation of the DNA dendrimer complex and transfection of Hs27 cells were performed according to the procedure described in Example 17.

After transfection, 0.1 ml of 1N HCl were added to 0.5 ml of the culture broth, mixed thoroughly, and incubated for 10 minutes at room temperature. Then, the mixture was neutralised by adding 0.1 ml of 1.2M NaOH/0.5M HEPES (pH 7-7.6) and stirred thoroughly. Supernatant was collected and used to assay the therapeutic protein. The P4HA1 protein was assayed by enzyme-linked immunosorbent assay (ELISA) using the Human P4HA1 ELISA Kit (Sandwich ELISA) (LifeSpan BioSciences, LS-F12242-1) according to the manufacturer's method with optical density detection using ChemWell Automated EIA and Chemistry Analyser (Awareness Technology Inc., USA).

To measure the numerical value of concentration, the calibration curve constructed using the reference samples from the kit with known concentrations of P4HA1 protein was used. The sensitivity was at least 625 pg/ml, measurement range—from 625 pg/ml to 40000 pg/ml. R-3.0.2 was used for the statistical treatment of the results and data visualization (https://www.r-project.org/). Drawings resulting from the assay are shown in FIG. 12.

FIG. 12 shows that the transfection of Hs27 human foreskin fibroblast cell line with gene therapy DNA vector VTvaf17-P4HA1 results in increased P4HA1 protein concentration compared to reference samples, which confirms the ability of the vector to penetrate eukaryotic cells and express the P4HA1 gene at the protein level. The presented results also confirm the practicability of use of gene therapy DNA vector VTvaf17-P4HA1 in order to increase the expression level of P4HA1 gene in eukaryotic cells.

### EXAMPLE 20

Proof of the efficiency and practicability of use of gene therapy DNA vector VTvaf17-P4HA2 carrying the P4HA2 gene in order to increase the expression of P4HA2 protein in mammalian cells.

Changes in the P4HA2 protein concentration in the lysate of Hs27 human foreskin fibroblast cell line (ATCC CRL-1634) were assessed after transfection of these cells with gene therapy DNA vector VTvaf17-P4HA2 carrying the human P4HA2 gene. Cells were cultured as described in Example 12.

The 6th generation SuperFect Transfection Reagent (Qiagen, Germany) was used for transfection. The aqueous dendrimer solution without DNA vector (A) and DNA vector VTvaf17 devoid of cDNA of P4HA2 gene (B) were used as a reference, and DNA vector VTvaf17-P4HA2 carrying the human P4HA2 gene (C) was used as the transfected agent. Preparation of the DNA dendrimer complex and transfection of Hs27 cells were performed according to the procedure described in Example 17.

After transfection, 0.1 ml of 1N HCl were added to 0.5 ml of the culture broth, mixed thoroughly, and incubated for 10 minutes at room temperature. Then, the mixture was neutralised by adding 0.1 ml of 1.2M NaOH/0.5M HEPES (pH 7-7.6) and stirred thoroughly. Supernatant was collected and used to assay the therapeutic protein.

The P4HA2 protein was assayed by enzyme-linked immunosorbent assay (ELISA) using the Human P4HA2 ELISA Kit (Sandwich ELISA) (LifeSpan BioSciences, LS-F33689-1) with optical density detection using ChemWell Automated EIA and Chemistry Analyser (Awareness Technology Inc., USA).

To measure the numerical value of concentration, the calibration curve constructed using the reference samples from the kit with known concentrations of P4HA2 protein was used. The sensitivity was at least 469 pg/ml, measurement range—from 780 pg/ml to 50000 pg/ml. R-3.0.2 was used for the statistical treatment of the results and data visualization (https://www.r-project.org/). Drawings resulting from the assay are shown in FIG. 13.

FIG. 13 shows that the transfection of Hs27 human foreskin fibroblast cell line with gene therapy DNA vector Vtvaf17-P4HA2 results in increased P4HA2 protein concentration compared to reference samples, which confirms the ability of the vector to penetrate eukaryotic cells and express the P4HA2 gene at the protein level. The presented results also confirm the practicability of use of gene therapy DNA vector Vtvaf17-P4HA2 in order to increase the expression level of P4HA2 gene in eukaryotic cells.

### EXAMPLE 21

Proof of the efficiency and practicability of use of gene therapy DNA vector VTvaf17-COL7A1 carrying the COL7A1 gene in order to increase the expression of COL7A1 protein in mammalian cells.

The change in the COL7A1 protein concentration in the lysate of HT 297.T human fibroblasts (ATCC® CRL-7782™) was assessed after transfection of these cells with DNA vector Vtvaf17-COL7A1 carrying the human COL7A1 gene. Cells were grown as described in Example 13.

The 6th generation SuperFect Transfection Reagent (Qiagen, Germany) was used for transfection. The aqueous dendrimer solution without DNA vector (A) and DNA vector Vtvaf17 devoid of cDNA of COL7A1 gene (B) were used as a reference, and DNA vector Vtvaf17-COL7A1 carrying the human COL7A1 gene (C) was used as the transfected agent. Preparation of the DNA dendrimer complex and transfection of HT 297.T cells were performed according to the procedure described in Example 17.

After transfection, 0.1 ml of 1N HCl were added to 0.5 ml of the culture broth, mixed thoroughly, and incubated for 10 minutes at room temperature. Then, the mixture was neutralised by adding 0.1 ml of 1.2M NaOH/0.5M HEPES (pH 7-7.6) and stirred thoroughly. Supernatant was collected and used to assay the therapeutic protein. The COL7A1 protein was assayed by enzyme-linked immunosorbent assay (ELISA) using the Human COL7A1/Collagen VII ELISA Kit (Sandwich ELISA) (LifeSpan BioSciences, LS-F11164-1) according to the manufacturer's method with optical density detection using ChemWell Automated EIA and Chemistry Analyser (Awareness Technology Inc., USA).

To measure the numerical value of concentration, the calibration curve constructed using the reference samples from the kit with known concentrations of COL7A1 protein was used. The sensitivity was at least 156 pg/ml, measurement range—from 156 pg/ml to 10000 pg/ml. R-3.0.2 was used for the statistical treatment of the results and data visualization (https://www.r-project.org/). Drawings resulting from the assay are shown in FIG. 14.

FIG. 14 shows that the transfection of HT 297.T human fibroblast cells with gene therapy DNA vector VTvaf17-COL7A1 results in increased COL7A1 protein concentration compared to reference samples, which confirms the ability of the vector to penetrate eukaryotic cells and express the COL7A1 gene at the protein level. The presented results also confirm the practicability of use of gene therapy DNA vector VTvaf17-COL7A1 in order to increase the expression level of the COL7A1 gene in eukaryotic cells.

### EXAMPLE 22

Proof of the efficiency and practicability of use of gene therapy DNA vector VTvaf17-CLCA2 carrying the CLCA2 gene in order to increase the expression of CLCA2 protein in mammalian cells.

The change in the CLCA2 protein concentration in the lysate of HT 297.T human fibroblasts (ATCC® CRL-7782™) was assessed after transfection of these cells with DNA vector VTvaf17-CLCA2 carrying the human CLCA2 gene. Cells were cultured as described in Example 14.

The 6th generation SuperFect Transfection Reagent (Qiagen, Germany) was used for transfection. The aqueous dendrimer solution without DNA vector (A) and DNA vector VTvaf17 devoid of cDNA of CLCA2 gene (B) were used as a reference, and DNA vector VTvaf17-CLCA2 carrying the human CLCA2 gene (C) was used as the transfected agent. Preparation of the DNA dendrimer complex and transfection of HT 297.T cells were performed according to the procedure described in Example 17.

After transfection, 0.1 ml of 1N HCl were added to 0.5 ml of the culture broth, mixed thoroughly, and incubated for 10 minutes at room temperature. Then, the mixture was neutralised by adding 0.1 ml of 1.2M NaOH/0.5M HEPES (pH 7-7.6) and stirred thoroughly. Supernatant was collected and used to assay the therapeutic protein. The CLCA2 protein was assayed by enzyme-linked immunosorbent assay (ELISA) using the Human Calcium activated chloride channel regulator 2 (CLCA2) ELISA Kit (MyBioSource, MB S7242681) according to the manufacturer's method with optical density detection using ChemWell Automated EIA and Chemistry Analyser (Awareness Technology Inc., USA).

To measure the numerical value of concentration, the calibration curve constructed using the reference samples from the kit with known concentrations of CLCA2 protein was used. The sensitivity was at least 1 pg/ml, measurement range—from 50 pg/ml to 1000 pg/ml. R-3.0.2 was used for the statistical treatment of the results and data visualization (https://www.r-project.org/). Drawings resulting from the assay are shown in FIG. 15.

FIG. 15 shows that the transfection of HT 297.T human fibroblast cells with gene therapy DNA vector VTvaf17-CLCA2 results in increased CLCA2 protein concentration compared to reference samples, which confirms the ability of the vector to penetrate eukaryotic cells and express the CLCA2 gene at the protein level. The presented results also confirm the practicability of use of gene therapy DNA vector VTvaf17-CLCA2 in order to increase the expression level of CLCA2 gene in eukaryotic cells.

### EXAMPLE 23

Proof of the efficiency and practicability of use of gene therapy DNA vector VTvaf17-ELN carrying the ELN gene in order to increase the expression of ELN protein in mammalian cells.

The change in the ELN protein concentration in the cell lysate of HEKa epidermal keratinocyte cell culture (ATCC PCS-200-011) was assessed after transfection of these cells with the DNA vector VTvaf17-ELN carrying the human ELN gene. Cells were cultured as described in Example 15.

The 6th generation SuperFect Transfection Reagent (Qiagen, Germany) was used for transfection. The aqueous dendrimer solution without DNA vector (A) and DNA vector VTvaf17 devoid of cDNA of ELN gene (B) were used as a reference, and DNA vector VTvaf17-ELN carrying the human ELN gene (C) was used as the transfected agent. Preparation of the DNA dendrimer complex and transfection of HEKa cells were performed according to the procedure described in Example 17.

After transfection, 0.1 ml of 1N HCl were added to 0.5 ml of the culture broth, mixed thoroughly, and incubated for 10 minutes at room temperature. Then, the mixture was neutralised by adding 0.1 ml of 1.2M NaOH/0.5M HEPES (pH 7-7.6) and stirred thoroughly. Supernatant was collected and used to assay the therapeutic protein.

The ELN protein was assayed by enzyme-linked immunosorbent assay (ELISA) using the Elastin ELISA Kit (Reddot Biotech, RD-ELN-Ra) according to the manufacturer's method with optical density detection using ChemWell Automated EIA and Chemistry Analyser (Awareness Technology Inc., USA).

To measure the numerical value of concentration, the calibration curve constructed using the reference samples from the kit with known concentrations of ELN protein was used. The sensitivity was at least 12.7 pg/ml, measurement range—from 31.25 pg/ml to 2000 pg/ml. R-3.0.2 was used for the statistical treatment of the results and data visualization (https://www.r-project.org/). Diagrams resulting from the assay are shown in FIG. 16.

FIG. 16 shows that the transfection of HEKa epidermal keratinocyte cell culture with gene therapy DNA vector VTvaf17-ELN results in increased ELN protein concentration compared to reference samples, which confirms the ability of the vector to penetrate eukaryotic cells and express the ELN gene at the protein level. The presented results also confirm the practicability of use of gene therapy DNA vector VTvaf17-ELN in order to increase the expression level of ELN gene in eukaryotic cells.

### EXAMPLE 24

Proof of the efficiency and practicability of use of gene therapy DNA vector VTvaf17-PLOD1 carrying the PLOD1 gene in order to increase the expression of PLOD1 protein in mammalian cells.

The change in the PLOD1 protein concentration in the cell lysate of HEMa epidermal melanocyte cell culture (ATCC® PCS200-013™) was assessed after transfection of these cells with the DNA vector VTvaf17-PLOD1 carrying the human PLOD1 gene. Cells were cultured as described in Example 16.

The 6th generation SuperFect Transfection Reagent (Qiagen, Germany) was used for transfection. The aqueous dendrimer solution without DNA vector (A) and DNA vector VTvaf17 devoid of cDNA of PLOD1 gene (B) were used as a reference, and DNA vector VTvaf17-PLOD1 carrying the human PLOD1 gene (C) was used as the transfected agent. Preparation of the DNA dendrimer complex and transfection of HEMa cells were performed according to the procedure described in Example 17.

After transfection, 0.1 ml of 1N HCl were added to 0.5 ml of the culture broth, mixed thoroughly, and incubated for 10 minutes at room temperature. Then, the mixture was neutralised by adding 0.1 ml of 1.2M NaOH/0.5M HEPES (pH 7-7.6) and stirred thoroughly. Supernatant was collected and used to assay the therapeutic protein.

The PLOD1 protein was assayed by enzyme-linked immunosorbent assay (ELISA) using the Human PLOD/PLOD1 ELISA Kit (Sandwich ELISA) (LifeSpan BioSciences, LS-F9705-1) according to the manufacturer's method with optical density detection using ChemWell Automated EIA and Chemistry Analyser (Awareness Technology Inc., USA).

To measure the numerical value of concentration, the calibration curve constructed using the reference samples from the kit with known concentrations of PLOD1 protein was used. The sensitivity was at least 66 pg/ml, measurement range—from 156 pg/ml to 10000 pg/ml. R-3.0.2 was used for the statistical treatment of the results and data visualization (https://www.r-project.org/). Diagrams resulting from the assay are shown in FIG. 17.

FIG. 17 shows that the transfection of HEMa human epidermal melanocyte cell culture with gene therapy DNA vector VTvaf17-PLOD1 results in increased PLOD1 protein concentration compared to reference samples, which confirms the ability of the vector to penetrate eukaryotic cells and express the PLOD1 gene at the protein level. The presented results also confirm the practicability of use of gene therapy DNA vector VTvaf17-PLOD1 in order to increase the expression level of PLOD1 gene in eukaryotic cells.

### EXAMPLE 25

Proof of the efficiency and practicability of use of gene therapy DNA vector VTvaf17-P4HA2 carrying the P4HA2 gene in order to increase the expression of P4HA2 protein in human tissues.

To prove the efficiency of gene therapy DNA vector VTvaf17-P4HA2 carrying the therapeutic gene, namely the P4HA2 gene, and practicability of its use, changes in P4HA2 protein concentration in human skin upon injection of gene therapy DNA vector VTvaf17-P4HA2 carrying the human P4HA2 gene were assessed.

To analyse changes in the P4HA2 protein concentration, gene therapy DNA vector VTvaf17-P4HA2 carrying the P4HA2 gene was injected into the forearm skin of three patients with concurrent injection of a placebo being gene therapy DNA vector VTvaf17 devoid of cDNA of P4HA2 gene.

Patient 1, woman, 44 y.o. (P1); Patient 2, woman, 61 y.o. (P2); Patient 3, man, 50 y.o. (P3). Polyethyleneimine Transfection reagent cGMP grade in-vivo-jetPEI (Polyplus Transfection, France) was used as a transport system. Gene therapy DNA vector VTvaf17-P4HA2 containing cDNA of P4HA2 gene and gene therapy DNA vector VTvaf17 used as a placebo not containing cDNA of P4HA2 gene were dissolved in sterile nuclease-free water. To obtain a gene construct, DNA-cGMP grade in-vivo-jetPEI complexes were prepared according to the manufacturer recommendations.

Gene therapy DNA vector VTvaf17 (placebo) and gene therapy DNA vector VTvaf17-P4HA2 carrying the P4HA2 gene were injected in the quantity of 1mg for each genetic construct using the tunnel method with a 30G needle to the depth of 3mm. The injectate volume of gene therapy DNA vector VTvaf17 (placebo) and gene therapy DNA vector VTvaf17-P4HA2 carrying the P4HA2 gene was 0.3 ml for each genetic construct. The points of injection of each genetic construct were located at 8 to 10 cm intervals at the forearm site.

The biopsy samples were taken on the 2nd day after the injection of the genetic constructs of gene therapy DNA vectors. The biopsy samples were taken from the patients' skin in the site of injection of gene therapy DNA vector VTvaf17-P4HA2 carrying the P4HA2 gene (I), gene therapy DNA vector VTvaf17 (placebo) (II), and from intact skin (III) using the skin biopsy device Epitheasy 3.5 (Medax SRL, Italy). The skin of patients in the biopsy site was preliminarily rinsed with sterile saline and anaesthetised with a lidocaine solution. The biopsy sample size was ca. 10 mm3, and the weight was approximately 11 mg. The sample was placed in a buffer solution containing 50 mM of Tris-HCl, pH 7.6, 100 mM of NaCl, 1 mM of EDTA, and 1 mM of phenylmethylsulfonyl fluoride, and homogenised to obtain a homogenised suspension. The suspension was then centrifuged for 10 minutes at 14,000 g. Supernatant was collected and used to assay the therapeutic protein by enzyme-linked immunosorbent assay (ELISA) using the Human P4HA2 ELISA Kit (Sandwich ELISA) (LifeSpan BioSciences, LS-F33689-1) according to the manufacturer's method with optical density detection using ChemWell Automated EIA and Chemistry Analyser (Awareness Technology Inc., USA).

To measure the numerical value of concentration, the calibration curve constructed using the reference samples from the kit with known concentrations of P4HA2 protein was used. The sensitivity was at least 469 pg/ml, measurement range—from 780 pg/ml to 50000 pg/ml. R-3.0.2 was used for the statistical treatment of the results and data visualization (https://www.r-project.org/). Drawings resulting from the assay are shown in FIG. 18.

FIG. 18 shows the increased P4HA2 protein concentration in the skin of all three patients in the injection site of gene therapy DNA vector VTvaf17-P4HA2 carrying the human P4HA2 therapeutic gene compared to the P4HA2 protein concentration in the injection site of gene therapy DNA vector VTvaf17 (placebo) devoid of the human P4HA2 gene, which indicates the efficiency of gene therapy DNA vector VTvaf17-P4HA2 and confirms the practicability of its use, in particular upon intracutaneous injection of gene therapy DNA vector in human tissues.

### EXAMPLE 26

Proof of the efficiency and practicability of use of gene therapy DNA vector VTvaf17-P4HA1 carrying the P4HA1 gene in order to increase the expression of P4HA1 protein in human tissues.

To prove the efficiency of gene therapy DNA vector VTvaf17-P4HA1 carrying the P4HA1 therapeutic gene and practicability of its use, the change in the P4HA1 protein concentration in human skin upon injection of gene therapy DNA vector VTvaf17-P4HA1 carrying the therapeutic gene, namely the human P4HA1 gene, was assessed.

To analyse changes in the concentration of P4HA1 protein, gene therapy DNA vector VTvaf17-P4HA1 carrying the P4HA1 gene with transport molecule was injected into the skin of three patients with concurrent injection of a placebo being gene therapy DNA vector VTvaf17 devoid of cDNA of P4HA1 gene with transport molecule.

Patient 1, man, 59 y.o. (P1); Patient 2, woman, 56 y.o. (P2); Patient 3, man, 58 y.o. (P3). Polyethyleneimine Transfection reagent cGMP grade in-vivo-jetPEI (Polyplus Transfection, France) was used as a transport system; sample preparation was carried out in accordance with the manufacturer's recommendations.

Gene therapy DNA vector VTvaf17 (placebo) and gene therapy DNA vector VTvaf17-P4HA1 carrying the P4HA1 gene were injected in the quantity of 1mg for each genetic construct using the tunnel method with a 30G needle to the depth of around 1 mm. The injectate volume of gene therapy DNA vector VTvaf17 (placebo) and gene therapy DNA vector VTvaf17-P4HA1 carrying the P4HA1 gene was 0.3 ml for each genetic construct. The points of introduction of each of the genetic constructs were located at 5 cm from each other.

The biopsy samples were taken on the 2nd day after the injection of the genetic constructs of gene therapy DNA vectors. The biopsy samples were taken from the patients' skin in the site of injection of gene therapy DNA vector VTvaf17-P4HA1 carrying the P4HA1 gene (I), gene therapy DNA vector VTvaf17 (placebo) (II), and from intact skin (III) using the skin biopsy device. The skin of patients in the biopsy site was preliminarily rinsed with sterile saline and anaesthetised with a lidocaine solution. The biopsy sample size was ca. 10 mm3, and the weight was approximately 11 mg. The sample was placed in a buffer solution containing 50 mM of Tris-HCl, pH 7.6, 100 mM of NaCl, 1 mM of EDTA, and 1 mM of phenylmethylsulfonyl fluoride, and homogenised to obtain a homogenised suspension. The suspension was then centrifuged for 10 minutes at 14,000 g. Supernatant was collected and used to assay the therapeutic protein.

The P4HA1 protein was assayed by enzyme-linked immunosorbent assay (ELISA) using the P4HA1 ELISA Kit (Sandwich ELISA) (LifeSpan BioSciences, LS-F12242-1) according to the manufacturer's method with optical density detection using ChemWell Automated EIA and Chemistry Analyser (Awareness Technology Inc., USA). The sensitivity is 0.625 ng/ml, measurement range—0.625-40 ng/ml.

To measure the numerical value of concentration, the calibration curve constructed using the reference samples from the kit with known concentrations of P4HA1 protein was used. The sensitivity was at least 625 pg/ml, measurement range—from 625 pg/ml to 40000 pg/ml. R-3.0.2 was used for the statistical treatment of the results and data visualization (https://www.r-project.org/). Drawings resulting from the assay are shown in FIG. 19.

FIG. 19 shows the increased P4HA1 protein concentration in the skin of all three patients in the injection site of gene therapy DNA vector VTvaf17-P4HA1 carrying the therapeutic gene, namely the human P4HA1 gene compared to the P4HA1 protein concentration in the injection site of gene therapy DNA vector VTvaf17 (placebo) devoid of the human P4HA1 gene, which indicates the efficiency of gene therapy DNA vector VTvaf17-P4HA1 and confirms the practicability of its use, in particular upon intracutaneous injection of gene therapy DNA vector in human tissues.

### EXAMPLE 27

Proof of the efficiency and practicability of combined use of gene therapy DNA vector VTvaf17-COL7A1 carrying the COL7A1 gene, gene therapy DNA vector VTvaf17-CLCA2 carrying the CLCA2 gene, gene therapy DNA vector VTvaf17-ELN carrying the ELN gene, and gene therapy DNA vector VTvaf17-PLOD1 carrying the PLOD1 gene for the increase of expression level of COL7A1, CLCA2, ELN, and PLOD1 proteins in human tissues.

To prove the efficiency of gene therapy DNA vector VTvaf17-COL7A1 carrying the COL7A1 gene, gene therapy DNA vector VTvaf17-CLCA2 carrying the CLCA2 gene, gene therapy DNA vector VTvaf17-ELN carrying the ELN gene, and gene therapy DNA vector VTvaf17-PLOD1 carrying the PLOD1 gene and practicability of its use, the change in the COL7A1, CLCA2, ELN, and PLOD1 protein concentration in human skin with concurrent injection of a mixture of gene therapy DNA vector VTvaf17-COL7A1 carrying the COL7A1 gene, gene therapy DNA vector VTvaf17-CLCA2 carrying the CLCA2 gene, gene therapy DNA vector VTvaf17-ELN carrying the ELN gene, and gene therapy DNA vector VTvaf17-PLOD1 carrying the PLOD1 gene was assessed.

To analyse changes in the COL7A1, CLCA2, ELN, and PLOD1 protein concentration, a mixture of gene therapy DNA vector VTvaf17-COL7A1 carrying the COL7A1 gene, gene therapy DNA vector VTvaf17-CLCA2 carrying the CLCA2 gene, gene therapy DNA vector VTvaf17-ELN carrying the ELN gene, and gene therapy DNA vector VTvaf17-PLOD1 carrying the PLOD1 gene was injected into the forearm skin of three patients with concurrent injection of a placebo being gene therapy DNA vector VTvaf17 devoid of cDNA of COL7A1, CLCA2, ELN and PLOD1 gene.

Patient 1, woman, 38 y.o. (P1); Patient 2, man, 48 y.o. (P2); Patient 3, man, 52 y.o. (P3). Polyethyleneimine Transfection reagent cGMP grade in-vivo-jetPEI (Polyplus Transfection, France) was used as a transport system. A mixture (in the ratio of 1:1:1:1) of gene therapy DNA vector VTvaf17-COL7A1 carrying the COL7A1 gene, gene therapy DNA vector VTvaf17-CLCA2 carrying the CLCA2 gene, gene therapy DNA vector VTvaf17-ELN carrying the ELN gene, and gene therapy DNA vector VTvaf17-PLOD1 carrying the PLOD1 gene and gene therapy DNA vector VTvaf17 used as a placebo that does not contain the cDNA of COL7A1, CLCA2, ELN, and PLOD1 genes each dissolved in sterile nuclease-free water. To obtain a gene construct,

DNA-cGMP grade in-vivo-jetPEI complexes were prepared according to the manufacturer recommendations.

Gene therapy DNA vector VTvaf17 (placebo) and a mixture of gene therapy DNA vector VTvaf17-COL7A1, gene therapy DNA vector VTvaf17-CLCA2, gene therapy DNA vector VTvaf17-ELN, and gene therapy DNA vector VTvaf17-PLOD1 were injected in the quantity of 4 mg for each genetic construct using the tunnel method with a 30G needle to the depth of 3 mm. The injectate volume of gene therapy DNA vector VTvaf17 (placebo) and a mixture of gene therapy DNA vector VTvaf17-COL7A1, gene therapy DNA vector VTvaf17-CLCA2, gene therapy DNA vector VTvaf17-ELN, and gene therapy DNA vector VTvaf17-PLOD1 was 1.2 ml for each genetic construct. The points of injection of each genetic construct were located at 8 to 10 cm intervals at the forearm skin site.

The biopsy samples were taken on the 2nd day after the injection of gene therapy DNA vectors. The biopsy samples were taken from the patients' skin in the site of injection of a mixture of gene therapy DNA vector VTvaf17-COL7A1 carrying the COL7A1 gene, gene therapy DNA vector VTvaf17-CLCA2 carrying the CLCA2 gene, gene therapy DNA vector VTvaf17-ELN carrying the ELN gene, and gene therapy DNA vector VTvaf17-PLOD1 carrying the PLOD1 gene (I), gene therapy DNA vector VTvaf17 (placebo) (II), and from intact skin (III) using the skin biopsy device Epitheasy 3.5 (Medax SRL, Italy). The skin of patients in the biopsy site was preliminarily rinsed with sterile saline and anaesthetised with a lidocaine solution. The biopsy sample size was ca. 10 mm3, and the weight was approximately 11 mg. The sample was placed in a buffer solution containing 50 mM of Tris-HCl, pH 7.6, 100 mM of NaCl, 1 mM of EDTA, and 1 mM of phenylmethylsulfonyl fluoride, and homogenised to obtain a homogenised suspension. The suspension was then centrifuged for 10 minutes at 14,000 g. Supernatant was collected and used to assay the therapeutic proteins as described in Example 21 (quantification of COL7A1 protein), Example 22 (quantification of CLCA2 protein), Example 23 (quantification of ELN protein), and Example 24 (quantification of PLOD1 protein).

To measure the numerical value of concentration, the calibration curve constructed using the reference samples from each kit with known concentrations of COL7A1, CLCA2, ELN, and PLOD1 protein was used. R-3.0.2 was used for the statistical treatment of the results and data visualization (https://www.r-project.org/). Drawings resulting from the assay are shown in FIG. 20.

FIG. 20 shows an increase in the concentration of COL7A1, CLCA2, ELN, and PLOD1 protein in the skin of all three patients in the injection site of a mixture of gene therapy DNA vector VTvaf17-COL7A1 carrying the COL7A1 gene, gene therapy DNA vector VTvaf17-CLCA2 carrying the CLCA2 gene, gene therapy DNA vector VTvaf17-ELN carrying the ELN gene, and gene therapy DNA vector VTvaf17-PLOD1 carrying the human PLOD1 gene, compared to the COL7A1, CLCA2, ELN, and PLOD1 protein concentration in the injection site of gene therapy DNA vector VTvaf17 (placebo) devoid of the human COL7A1, CLCA2, ELN, and PLOD1 gene, which indicates the efficiency of gene therapy DNA vector VTvaf17-COL7A1, gene therapy DNA vector VTvaf17-CLCA2, gene therapy DNA vector VTvaf17-ELN, and gene therapy DNA vector VTvaf17-PLOD1 and confirms the practicability of its use, in particular upon intracutaneous injection of gene therapy DNA vector in human tissues.

### EXAMPLE 28

Proof of the efficiency of gene therapy DNA vector VTvaf17-COL1A2 carrying the COL1A2 gene and practicability of its use in order to increase the expression level of the COL1A2 protein in human tissues by introducing autologous fibroblasts transfected with gene therapy DNA vector VTvaf17-COL1A2.

To confirm the efficiency of gene therapy DNA vector VTvaf17-COL1A2 carrying the COL1A2 gene and practicability of its use, changes in the COL1A2 protein level in human skin upon injection of patient's skin with autologous fibroblast culture of the same patient transfected with gene therapy DNA vector VTvaf17-COL1A2 were assessed.

The appropriate autologous fibroblast culture transfected with the gene therapy DNA vector VTvaf17-COL1A2 carrying the COL1A2 gene was injected into the patient's forearm skin with concurrent injection of a placebo in the form of autologous fibroblast culture transfected with gene therapy DNA vector VTvaf17 not carrying the COL1A2 gene.

The human primary fibroblast culture was isolated from the patient skin biopsy specimens. Biopsy specimens of the skin from the area protected by ultraviolet, namely behind the ear or on the inner lateral side of the elbow, were taken using the skin biopsy device Epitheasy 3.5 (Medax SRL, Italy). The biopsy sample was ca. 10mm and ca. 11 mg. The patient's skin was preliminarily rinsed with sterile saline and anaesthetised with a lidocaine solution. The primary cell culture was cultivated at 37° C. in the presence of 5% CO2, in the DMEM medium with 10% fetal bovine serum and 100 U/ml of ampicillin. The passage and change of culture medium were performed every 2 days. Total duration of culture growth did not exceed 25-30 days. Then an aliquot of 5×10⁴ cells was taken from the cell culture. The patient's fibroblast culture was transfected with the gene therapy DNA vector VTvaf17-COL1A2 carrying the COL1A2 gene and placebo, i.e. VTvaf17 vector not carrying the COL1A2 therapeutic gene.

The transfection was carried out using a cationic polymer such as polyethyleneimine JETPEI (Polyplus transfection, France), according to the manufacturer's instructions. The cells were cultured for 72 hours and then injected into the patient. Injection of autologous fibroblast culture of the patient transfected with gene therapy DNA vector VTvaf17-COL1A2 and autologous fibroblast culture of the patient transfected with gene therapy DNA vector VTvaf17 as a placebo was performed in the forearm using the tunnel method with a 13 mm long 30G needle to the depth of approximately 3 mm. The concentration of the modified autologous fibroblasts in the injected suspension was approximately 5 mln cells per 1 ml of the suspension, the dose of the injected cells did not exceed 15 mln. The points of injection of the autologous fibroblast culture were located at 8 to 10cm intervals.

Biopsy samples were taken on the 4th day after the injection of autologous fibroblast culture transfected with the gene therapy DNA vector VTvaf17-COL1A2 carrying the therapeutic gene, namely COL1A2 gene, and placebo. Biopsy was taken from the patient's skin in the site of injection of autologous fibroblast culture transfected with gene therapy DNA vector VTvaf17-COL1A2 carrying the therapeutic gene, namely COL1A2 gene (C), autologous fibroblast culture transfected with gene therapy DNA vector VTvaf17 not carrying the COL1A2 therapeutic gene (placebo) (B), as well as from intact skin site (A) using the skin biopsy device Epitheasy 3.5 (Medax SRL, Italy). The skin of patients in the biopsy site was preliminarily rinsed with sterile saline and anaesthetised with a lidocaine solution. The biopsy sample size was ca. 10 mm3, and the weight was approximately 11 mg. The sample was placed in a buffer solution containing 50 mM of Tris-HCl, pH 7.6, 100 mM of NaCl, 1 mM of EDTA, and 1mM of phenylmethylsulfonyl fluoride, and homogenised to obtain a homogenised suspension. The suspension was then centrifuged for 10 minutes at 14,000g. Supernatant was collected and used to assay the therapeutic COL1A2 protein as described in Example 18.

Drawings resulting from the assay are shown in FIG. 21.

FIG. 21 shows the increased concentration of COL1A2 protein in the area of the patient's skin in the injection site of autologous fibroblast culture transfected with the gene therapy DNA vector VTvaf17-COL1A2 carrying the COL1A2 gene compared to the COL1A2 protein concentration in the injection site of autologous fibroblast culture transfected with the gene therapy DNA vector VTvaf17 that does not carry the COL1A2 gene (placebo), which indicates the efficiency of gene therapy DNA vector VTvaf17-COL1A2 and practicability of its use in order to increase the expression level of COL1A2 in human tissues, in particular upon injection of autologous fibroblasts transfected with the gene therapy DNA vector VTvaf17-COL1A2 into the skin.

### EXAMPLE 29

Proof of the efficiency and practicability of combined use of gene therapy DNA vector VTvaf17-COL1A1 carrying the COL1A1 gene, gene therapy DNA vector VTvaf17-COL1A2 carrying the COL1A2 gene, gene therapy DNA vector VTvaf17-P4HA1 carrying the P4HA1 gene, gene therapy DNA vector VTvaf17-P4HA2 carrying the P4HA2 gene in order to increase the expression level of the COL1A1, COL1A2, P4HA1, and P4HA2 proteins in mammalian tissues.

The change in the COL1A1, COL1A2, P4HA1, and P4HA2 protein concentration in the rat skin were assessed upon injection of a mixture of gene therapy vectors into three rats.

Polyethyleneimine Transfection reagent cGMP grade in-vivo-jetPEI (Polyplus Transfection, France) was used as a transport system. Equimolar mixture of gene therapy DNA vectors was dissolved in sterile nuclease-free water. To obtain a gene construct, DNA-cGMP grade in-vivo-jetPEI complexes were prepared according to the manufacturer recommendations. The injectate volume was 0.05 ml with a total quantity of DNA equal to 100 m. The solution was injected by tunnel method with a 33G needle to the depth of 0.5 mm in the site of preliminary epilated rat skin.

The biopsy samples were taken on the 2nd day after the injection of the gene therapy DNA vectors. The biopsy sample was taken from muscle sites in the region of injection of a mixture of gene therapy DNA vectors carrying the genes COL1A1, COL1A2, P4HA1, and P4HA2 (site I), gene therapy DNA vector VTvaf17 (placebo) (site II), as well as from the skin intact sites of animal (site III), using the skin biopsy device Epitheasy 3.5 (Medax SRL). The biopsy sample site was preliminarily rinsed with sterile saline and anaesthetised with a lidocaine solution. The biopsy sample size was ca. 10 mm3, and the weight was approximately 11 mg. Each sample was placed in a buffer solution containing 50 mM of Tris-HCl, pH 7.6, 100mM of NaCl, 1 mM of EDTA, and 1 mM of phenylmethylsulfonyl fluoride, and homogenised to obtain a homogenised suspension. The suspension was then centrifuged for 10 minutes at 14,000 g. Supernatant was collected and used to assay the therapeutic proteins as described in Example 17 (quantification of COL1A1 protein), Example 18 (quantification of COL1A2 protein), Example 19 (quantification of P4HA1 protein), and Example 20 (quantification of P4HA2 protein). Drawings resulting from the assay are shown in FIG. 22.

FIG. 22 demonstrates that there was an increase of COL1A1, COL1A2, P4HA1, and P4HA2 protein concentration in the all rats skin site (site I) where a mixture of gene therapy DNA vector VTvaf17-COL1A1 carrying the COL1A1 therapeutic gene, therapy DNA vector VTvaf17-COL1A2 carrying the COL1A2 therapeutic gene, gene therapy DNA vector VTvaf17-P4HA1 carrying the P4HA1 therapeutic gene, gene therapy DNA vector VTvaf17-P4HA2 carrying the P4HA2 therapeutic gene were injected compared to site II (placebo site) and site III (intact site). The obtained results show the efficiency of combined use of gene therapy DNA vector VTvaf17-COL1A1, gene therapy DNA vector VTvaf17-COL1A2, gene therapy DNA vector VTvaf17-P4HA1, and gene therapy DNA vector VTvaf17-P4HA2 and practicability of their use for the increase of the expression level of therapeutic proteins in mammalian tissues.

### EXAMPLE 30

Proof of the efficiency of gene therapy DNA vector VTvaf17-ELN carrying the ELN gene and practicability of its use in order to increase the expression level of ELN protein in mammalian cells.

To prove the efficiency of gene therapy DNA vector VTvaf17-ELN carrying the ELN gene, changes in mRNA accumulation of the ELN therapeutic gene in BDF bovine dermal fibroblast cells (ScienCell, Cat. #B2300) 48 hours after their transfection with gene therapy DNA vector VTvaf17-ELN carrying the human ELN gene were assessed.

Bovine dermal fibroblast cells BDF were grown in the FM-2 medium (ScienCell, Cat. #2331). Transfection with gene therapy DNA vector VTvaf17-ELN carrying the human ELN gene and DNA vector VTvaf17 not carrying the human ELN gene (reference), RNA extraction, reverse transcription reaction, PCR amplification, and data analysis were performed as described in Example 15. Bull/cow actin gene (ACT) listed in the GenBank database under number AH001130.2 was used as a reference gene. Positive control included amplicons from PCR on matrices represented by plasmids in known concentrations containing ELN and ACT gene sequences. Negative control included deionised water. Real-time quantification of the PCR products, i.e. ELN and ACT gene cDNAs obtained by amplification, was conducted using the Bio-Rad CFX Manager 2.1 software. (Bio-Rad, USA).

Diagrams resulting from the assay are shown in FIG. 23.

FIG. 23 shows that the level of specific mRNA of human ELN gene has grown massively as a result of transfection of bovine dermal fibroblast cells BDF with gene therapy DNA vector VTvaf17-ELN, which confirms the ability of the vector to penetrate eukaryotic cells and express the ELN gene at the mRNA level. The presented results confirm the practicability of use of gene therapy DNA vector VTvaf17-ELN in order to increase the expression level of ELN gene in mammalian cells.

### EXAMPLE 31

Escherichia coli strain SCS110-AF/VTvaf17-COL1A1, or Escherichia coli strain SCS110-AFNTvaf17-COL1A2, or Escherichia coli strain SCS110-AF/VTvaf17-P4HA1, or Escherichia coli strain SCS 110-AF/VTvaf17-P4HA2, or Escherichia coli strain SCS110-AFNTvaf17-COL7A1, or Escherichia coli strain SCS110-AFNTvaf17-CLCA2, or Escherichia coli SCS110-AF/VTvaf17-ELN, or Escherichia coli strain SCS110-AFNTvaf17-PLOD1 carrying the gene therapy DNA vector, and method of its production.

The construction of strain for the production of gene therapy DNA vector based on gene therapy DNA vector VTvaf17 carrying the therapeutic gene on an industrial scale selected from the group of the following genes: COL1A1, COL1A2, P4HA1, P4HA2, COL7A1, CLCA2, ELN and PLOD1 namely Escherichia coli strain SCS110-AFNTvaf17-COL1A1, or Escherichia coli strain SCS110-AF/VTvaf17-COL1A2, or Escherichia coli strain SCS110-AFNTvaf17-P4HA1, or Escherichia coli strain SCS110-AFNTvaf17-P4HA2, or Escherichia coli strain SCS110-AF/VTvaf1 7-COL7A1, or Escherichia coli strain SCS110-AF/VTvaf17-CLCA2, or Escherichia coli SCS110-AFNTvaf17-ELN, or Escherichia coli strain SCS110-AF/VTvaf17-PLOD1 carrying gene therapy DNA vector VTvaf17-COL1A1, or VTvaf17-COL1A2, or VTvaf17-P4HA1, or VTvaf17-P4HA2, or VTvaf17-COL7A1, or VTvaf17-CLCA2, or VTvaf17-ELN, or VTvaf17-PLOD1, respectively, for its production allowing for antibiotic-free selection involves making electrocompetent cells of Escherichia coli strain SCS110-AF and subjecting these cells to electroporation with gene therapy DNA vector VTvaf17-COL1A1, or gene therapy DNA vector VTvaf17-COL1A2, or gene therapy DNA vector VTvaf17-P4HA1, or gene therapy DNA vector VTvaf17-P4HA2, or gene therapy DNA vector VTvaf17-COL7A1, or gene therapy DNA vector VTvaf17-CLCA2, or gene therapy DNA vector VTvaf17-ELN, or gene therapy DNA vector VTvaf17-PLOD1. After that, the cells were poured into agar plates (Petri dishes) with a selective medium containing yeastrel, peptone, 6% sucrose, and 10μg/ml of chloramphenicol. At the same time, production of Escherichia coli strain SCS110-AF for the production of gene therapy DNA vector VTvaf17 or gene therapy DNA vectors based on it allowing for antibiotic-free positive selection involves constructing a 64 bp linear DNA fragment that contains regulatory element RNA-IN of transposon Tn10 allowing for antibiotic-free positive selection, a 1422 bp levansucrase gene sacB, the product of which ensures selection within a sucrose-containing medium, a 763 bp chloramphenicol resistance gene catR required for the selection of strain clones in which homologous recombination occurs, and two homologous sequences, 329 bp and 233 bp, ensuring homologous recombination in the region of gene recA concurrent with gene inactivation, and then the Escherichia coli cells are transformed by electroporation, and clones surviving in a medium containing 10 μg/ml of chloramphenicol are selected.

The obtained strains for production were included in the collection of the National Biological Resource Centre—Russian National Collection of Industrial Microorganisms (NBRC RNCIM), RF and NCIMB Patent Deposit Service, UK under the following registration numbers:

Escherichia coli strain SCS110-AFNTvaf17-COL1A1—registered at the Russian National Collection of Industrial Microorganisms under number B-13165, date of deposit 11.05.2018; INTERNATIONAL DEPOSITARY AUTHORITY No. NCIMB 43033, date of deposit 20.04.2018,

Escherichia coli strain SCS110-AFNTvaf17-COL1A2—registered at the Russian National Collection of Industrial Microorganisms under number B-13164, date of deposit 11.05.2018; INTERNATIONAL DEPOSITARY AUTHORITY No. NCIMB 43035, date of deposit 20.04.2018,

Escherichia coli strain SCS110-AF/VTvaf17-P4HA1—registered at the Russian National Collection of Industrial Microorganisms under number B-13384, date of deposit 14.12.2018; INTERNATIONAL DEPOSITARY AUTHORITY No. NCIMB 43311, date of deposit 13.12.2018,

Escherichia coli strain SCS110-AF/VTvaf17-P4HA2—registered at the Russian National Collection of Industrial Microorganisms under number B-13388, date of deposit 14.12.2018; INTERNATIONAL DEPOSITARY AUTHORITY No. NCIMB 43312, date of deposit 13.12.2018,

Escherichia coli strain SCS110-AF/VTvaf17-CLCA2—registered at the Russian National Collection of Industrial Microorganisms under number B-13385, date of deposit 14.12.2018; INTERNATIONAL DEPOSITARY AUTHORITY No. NCIMB 43308, date of deposit 13.12.2018,

Escherichia coli strain SCS110-AF/VTvaf17-ELN—registered at the Russian National Collection of Industrial Microorganisms under number B-13341, date of deposit 22.11.2018; INTERNATIONAL DEPOSITARY AUTHORITY No. NCIMB 43281, date of deposit 22.11.2018,

Escherichia coli strain SCS110-AF/VTvaf17-PLOD1—registered at the Russian National Collection of Industrial Microorganisms under number B-13387, date of deposit 14.12.2018; INTERNATIONAL DEPOSITARY AUTHORITY No. NCIMB 43313, date of deposit 13.12.2018.

### EXAMPLE 32

The method for scaling up of the gene therapy DNA vector based on gene therapy DNA vector VTvaf17 carrying the therapeutic gene selected from the group of COL1A1, COL1A2, P4HA1, P4HA2, COL7A1, CLCA2, ELN, and PLOD1 to an industrial scale.

To confirm the producibility and constructability on an industrial scale of gene therapy DNA vector VTvaf17-COL1A1 (SEQ ID No. 1), or VTvaf17-COL1A2 (SEQ ID No. 2), or VTvaf17-P4HA1,(SEQ ID No. 3), or VTvaf17-P4HA2 (SEQ ID No. 4), or VTvaf17-COL7A1 (SEQ ID No. 5), or VTvaf17-CLCA2 (SEQ ID No. 6), or VTvaf17-ELN, (SEQ ID No. 7), or VTvaf17-PLOD1 (SEQ ID No. 8), large-scale fermentation of Escherichia coli strain SCS110-AF/VTvaf17-COL1A1, or Escherichia coli SCS110-AFNTvaf17-COL1A2, or Escherichia coli strain SCS110-AFNTvaf17-P4HA1, or Escherichia coli strain SCS110-AF/VTvaf17-P4HA2, or Escherichia coli strain SCS110-AFNTvaf17-COL7A1, or Escherichia coli strain SCS110-AFNTvaf17-CLCA2, or Escherichia coli SCS110-AF/VTvaf17-ELN, or Escherichia coli strain SCS110-AFNTvaf17-PLOD1, each containing gene therapy DNA vector VTvaf17 carrying a region of the therapeutic gene, namely COL1A1, or COL1A2, or P4HA1, or P4HA2, or COL7A1, or CLCA2, or ELN, or PLOD1. Each Escherichia coli strain SCS110-AFNTvaf17-COL1A1, or Escherichia coli strain SCS110-AF/VTvaf17-COL1A2, or Escherichia coli strain SCS110-AFNTvaf17-P4HA1, or Escherichia coli strain SCS110-AFNTvaf17-P4HA2, or Escherichia coli strain SCS110-AF/VTvaf17-COL7A1, or Escherichia coli strain SCS110-AFNTvaf17-CLCA2, or Escherichia coli SCS110-AFNTvaf17-ELN, or Escherichia coli strain SCS110-AFNTvaf17-PLOD1 was produced on the basis of Escherichia coli strain SCS110-AF (Cell and Gene Therapy LLC, United Kingdom) as described in Example 31 by electroporation of competent cells of this strain with the gene therapy DNA vector VTvaf17-COL1A1, or VTvaf17-COL1A2, or VTvaf17-P4HA1, or VTvaf17-P4HA2, or VTvaf17-COL7A1, or VTvaf17-CLCA2, or VTvaf17-ELN, or VTvaf17-PLOD1 carrying the therapeutic gene, namely COL1A1, COL1A2, P4HA1, P4HA2, COL7A1, CLCA2, ELN, and PLOD1 with further inoculation of transformed cells into agar plates (Petri dishes) with a selective medium containing yeastrel, peptone, and 6% sucrose, and selection of individual clones.

Fermentation of Escherichia coli strain SCS110-AFNTvaf17-COL1A1 carrying gene therapy DNA vector VTvaf17-COL1A1 was performed in a 101 fermenter with subsequent extraction of gene therapy DNA vector VTvaf17-COL1A1.

For the fermentation of Escherichia coli strain SCS110-AFNTvaf17-COL1A1, medium containing the following ingredients per 101 of volume was prepared: 100g of tryptone and 50g of yeastrel (Becton Dickinson, USA); then the medium was diluted with water to 8800 ml and autoclaved at 121° C. for 20 minutes, and then 1200 ml of 50% (w/v) sucrose was added. After that, the seed culture of Escherichia coli strain SCS110-AFNTvaf17-COL1A1 was inoculated into a culture flask in the volume of 100 ml. The culture was incubated in an incubator shaker for 16 hours at 30° C. The seed culture was transferred to the Techfors S bioreactor (Infors HT, Switzerland) and grown to a stationary phase. The process was controlled by measuring optical density of the culture at 600 nm. The cells were pelleted for 30 minutes at 5,000-10,000 g. Supernatant was removed, and the cell pellet was re-suspended in 10% (by volume) phosphate buffered saline. The cells were centrifuged again for 30 minutes at 5,000-10,000 g. Supernatant was removed, a solution of 20 mM TrisCl, 1 mM EDTA, 200g/l sucrose, pH 8.0 was added to the cell pellet in the volume of 1000 ml, and the mixture was stirred thoroughly to a homogenised suspension. Then egg lysozyme solution was added to the final concentration of 100 μg/ml. The mixture was incubated for 20 minutes on ice while stirring gently. Then 2500 ml of 0.2M NaOH, 10 g/l sodium dodecyl sulphate (SDS) was added, the mixture was incubated for 10 minutes on ice while stirring gently, then 3500 ml of 3M sodium acetate, 2M acetic acid, pH 5-5.5 was added, and the mixture was incubated for 10 minutes on ice while stirring gently. The resulting sample was centrifuged for 20-30 minutes at 15,000 g or a greater value. The solution was decanted delicately, and residual precipitate was removed by passing through a coarse filter (filter paper). Then, RNase A (Sigma, USA) was added to the final concentration of 20 μg/ml, and the solution was incubated overnight for 16 hours at room temperature. The solution was then centrifuged for 20-30 minutes at 15,000 g and passed through a 0.45 μm membrane filter (Millipore, USA). Then, ultrafiltration was performed with a 100 kDa membrane (Millipore, USA) and the mixture was diluted to the initial volume with a buffer solution of 25 mM TrisCl, pH 7.0. This manipulation was performed three to four times. The solution was applied to the column with 250 ml of DEAE Sepharose HP (GE, USA), equilibrated with 25mM TrisCl, pH 7.0. After the application of the sample, the column was washed with three volumes of the same solution, and then gene therapy DNA vector VTvaf17-COL1A1 was eluted using a linear gradient of 25 mM TrisCl, pH 7.0, to obtain a solution of 25 mM TrisCl, pH 7.0, 1M NaCl, five times the volume of the column. The elution process was controlled by measuring optical density of the run-off solution at 260 nm. Chromatographic fractions containing gene therapy DNA vector VTvaf17-COL1A1 were joined together and subjected to gel filtration using Superdex 200 (GE, USA). The column was equilibrated with phosphate buffered saline. The elution process was controlled by measuring optical density of the run-off solution at 260 nm, and the fractions were analysed by agarose gel electrophoresis. The fractions containing gene therapy DNA vector VTvaf17-COL1A1 were joined together and stored at −20° C. To assess the process reproducibility, the indicated processing operations were repeated five times. All processing operations for Escherichia coli strain SCS110-AFNTvaf17-COL1A2, or Escherichia coli strain SCS110-AFNTvaf17-P4HA1, or Escherichia coli strain SCS110-AF/VTvaf17-P4HA2, or Escherichia coli strain SCS110-AFNTvaf17-COL7A1, or Escherichia coli strain SCS110-AFNTvaf17-CLCA2, or Escherichia coli SCS110-AF/VTvaf17-ELN, or Escherichia coli strain SCS110-AF/VTvaf17-PLOD1 were performed in a similar way.

The process reproducibility and quantitative characteristics of final product yield confirm the producibility and constructability of gene therapy DNA vector VTvaf17-COL1A1, or VTvaf17-COL1A2, or VTvaf17-P4HA1, or VTvaf17-P4HA2, or VTvaf17-COL7A1, or VTvaf17-CLCA2, or VTvaf17-ELN, or VTvaf17-PLOD1 on an industrial scale.

Thus, the produced gene therapy DNA vector containing the therapeutic gene can be used to deliver it to the cells of human beings and animals that experience reduced or insufficient expression of protein encoded by this gene, thus ensuring the desired therapeutic effect.

The purpose set in this invention, namely the construction of the gene therapy DNA vectors in order to increase the expression level of COL1A1, COL1A2, P4HA1, P4HA2, COL7A1, CLCA2, ELN, and PLOD1 genes that combine the following properties:

I) The effectiveness of increase of expression of therapeutic genes in eukaryotic cells due to the obtained gene therapy vectors with limited size of vector part,

II) Possibility of safe use in gene therapy of human beings and animals due to the absence of regulatory elements representing the nucleotide sequences of viral genomes and antibiotic resistance genes in the gene therapy DNA vector,

III) Producibility and constructability in the strains on an industrial scale,

IV) as well as the purpose of the construction of strains carrying these gene therapy DNA vectors for the production of these gene therapy DNA vectors is achieved, which is supported by the following examples:

for I—Example 1, 2, 3, 4, 5; 6; 7; 8; 9; 10; 11; 12; 13; 14; 15; 16; 17; 18, 19, 20, 21, 22, 23, 24, 25, 26, 27, 28, 29, 30;

for II—Example 1, 2, 3, 4, 5; 6; 7; 8; 9; 10; 11; 12; 13; 14; 15; 16; 17; 18, 19, 20, 21, 22, 23, 24, 25, 26, 27, 28, 29, 30;

for III—Example 1, 2, 3, 4, 5; 6; 7; 8, 31, 32;

for IV—Example 31, 32.

### INDUSTRIAL APPLICABILITY

All the examples listed above confirm the industrial applicability of the proposed gene therapy DNA vector based on gene therapy DNA vector VTvaf17 carrying the therapeutic gene selected from the group of COL1A1, COL1A2, P4HA1, P4HA2, COL7A1, CLCA2, ELN, and PLOD1 genes in order to increase the expression level of these therapeutic genes, Escherichia coli strain SCS110-AF/VTvaf17-COL1A1, or Escherichia coli strain SCS110-AF/VTvaf17-COL1A2, or Escherichia coli strain SCS110-AFNTvaf17-P4HA1, or Escherichia coli strain SCS110-AF/VTvaf17-P4HA2, or Escherichia coli strain SCS110-AF/VTvaf17-COL7A1, or Escherichia coli strain SCS110-AF/VTvaf17-CLCA2, or Escherichia coli SCS110-AF/VTvaf17-ELN, or Escherichia coli strain SCS110-AF/VTvaf17-PLOD1 carrying gene therapy DNA vector, and method of its production on an industrial scale.

### (1) LIST OF ABBREVIATIONS

VTvaf17—Gene therapy vector devoid of sequences of viral genomes and antibiotic resistance markers (vector therapeutic virus-antibiotic-free)
DNA—Deoxyribonucleic acid
cDNA—Complementary deoxyribonucleic acid
RNA—Ribonucleic acid
mRNA—Messenger ribonucleic acid
bp—base pair
PCR—Polymerase chain reaction
ml—millilitre, μl—microlitre
mm3—cubic millimetre
l—litre
μg—microgram
mg—milligram
g—gram
μM—micromol
mM—millimol
min—minute
s—second
rpm—rotations per minute
nm—nanometre
cm—centimetre
mW—milliwatt
RFU—Relative fluorescence unit
PBS—Phosphate buffered saline

### LIST OF REFERENCES

1. Bart G, Hämäläinen L, Rauhala L, Salonen P, Kokkonen M, Dunlop T W, Pehkonen P, Kumlin T, Tammi M I, Pasonen-Seppanen S, Tammi R H. rClca2 is associated with epidermal differentiation and is strongly downregulated by ultraviolet radiation. Br J Dermatol. 2014 Aug;171(2):376-87.

2. Connon C J, Yamasaki K, Kawasaki S, Quantock A J, Koizumi N, Kinoshita S. Calcium-activated chloride channel-2 in human epithelia. J Histochem Cytochem. 2004 Mar;52(3):415-8.

3. Craven et al., 1997

4. Draft Guideline on the quality, non-clinical and clinical aspects of gene therapy medicinal products, http://www.ema.europa.eu/docs/en_GB/document_library/Scientific_guideline/2015/05/WC500187020.pdf

5. Ewart A K, Jin W, Atkinson D, Morris C A, Keating M T. Supravalvular aortic stenosis associated with a deletion disrupting the elastin gene. J Clin Invest. 1994 March; 93(3): 1071-7.

6. Georgiadis C, Syed F, Petrova A, Abdul-Wahab A, Lwin S M, Farzaneh F, Chan L, Ghani S, Fleck R A, Glover L, McMillan J R, Chen M, Thrasher A J, McGrath J A, Di W L, Qasim W. Lentiviral Engineered Fibroblasts Expressing Codon-Optimized COL7A1 Restore Anchoring Fibrils in RDEB. J Invest Dermatol. 2016 January; 136(1): 284-92.

7. Goto M, Sawamura D, Ito K, Abe M, Nishie W, Sakai K, Shibaki A, Akiyama M, Shimizu H. Fibroblasts show more potential as target cells than keratinocytes in COL7A1 gene therapy of dystrophic epidermolysis bullosa. J Invest Dermatol. 2006 April; 126(4): 766-72.

8. Guideline on the quality, non-clinical and clinical aspects of gene therapy medicinal Products EMA/CAT/80183/2014

9. Hatzimichael E, Lo Nigro C, Lattanzio L, Syed N, Shah R, Dasoula A, Janczar K, Vivenza D, Monteverde M, Merlano M, Papoudou-Bai A, Bai M, Schmid P, Stebbing J, Bower M, Dyer M J, Karran L E, ElguetaKarstegl C, Farrell P J, Thompson A, Briasoulis E, Crook T. The collagen prolyl hydroxylases are novel transcriptionally silenced genes in lymphoma. Br J Cancer. 2012 Oct. 9; 107(8) :1423-32.

10. Hornstein B D, Roman D, Arevalo-Soliz L M, Engevik M A, Zechiedrich L. Effects of Circular DNA Length on Transfection Efficiency by Electroporation into HeLa Cells. Celia V, ed. PLoS ONE. 2016; 11(12): e0167537.

11. Jacków J, Titeux M, Portier S, Charbonnier S, Ganier C, Gaucher S, Hovnanian A. Gene-Corrected Fibroblast Therapy for Recessive Dystrophic Epidermolysis Bullosa using a Self-Inactivating COL7A1 Retroviral Vector. J Invest Dermatol. 2016 July; 136(7): 1346-1354.

12. Keene D R, Sakai L Y, Lunstrum G P, Morris N P, Burgeson R E. Type VII collagen forms an extended network of anchoring fibrils. J Cell Biol. 1987 March; 104(3): 611-21.

13. Kielty C M, Sherratt M J, Shuttleworth C A. Elastic fibres. J Cell Sci. 2002 Jul. 15; 115(Pt 14): 2817-28.

14. Labat-Robert J. Cell-matrix interactions, alteration with aging and age associated diseases. A review. Pathol Biol (Paris). 2001 May; 49(4): 349-52.

15. Li L, Petrovsky N. Molecular mechanisms for enhanced DNA vaccine immunogenicity. Expert Rev Vaccines. 2016; 15(3): 313-29

16. Li S H, Sun Z, Guo L, Han M, Wood M F, Ghosh N, Vitkin I A, Weisel R D, Li R K. Elastin overexpression by cell-based gene therapy preserves matrix and prevents cardiac dilation. J Cell Mol Med. 2012 October; 16(10): 2429-39.

17. Mairhofer J, Grabherr R. Rational vector design for efficient non-viral gene delivery: challenges facing the use of plasmid DNA. Mol Biotechnol. 2008.39(2): 97-104

18. Majka, Magdalena Janeczko, Jolanta Goździk, Danuta Jarocha, Aleksandra Auguściak-Duma, Joanna Witecka, Marta Lesiak, Halina Koryciak-Komarska, Aleksander L. Sieroń, Jacek Józef Pietrzyk, Cell therapy of a patient with type III osteogenesis imperfecta caused by mutation in COL1A2 gene and unstable collagen type I. Open Journal of Genetics, Vol.3 No. 1,2013

19. Mecklenbeck S, Compton S H, Mejia J E, Cervini R, Hovnanian A, Bruckner-Tuderman L, Barrandon Y. A microinjected COL7A1-PAC vector restores synthesis of intact procollagen VII in a dystrophic epidermolysis bullosa keratinocyte cell line. Hum Gene Ther. 2002 Sep. 1; 13(13): 1655-62.

20. Mencia Á, Chamorro C, Bonafont J, Duarte B, Holguin A, Illera N, Llames S G, Escamez M J, Hausser I, Del Rio M, Larcher F, Murillas R. Deletion of a Pathogenic Mutation-Containing Exon of COL7A1 Allows Clonal Gene Editing Correction of RDEB Patient Epidermal Stem Cells. Mol Ther Nucleic Acids. 2018 Jun. 1; 11: 68-78.

21. Napolitano F, Di Iorio V, Testa F, Tirozzi A, Reccia M G, Lombardi L, Farina O, Simonelli F, Gianfrancesco F, Di Iorio G, Melone M A B, Esposito T, Sampaolo S. Autosomal-dominant myopia associated to a novel P4HA2 missense variant and defective collagen hydroxylation. Clin Genet. 2018 May; 93(5): 982-991.

22. Naylor E C, Watson R E, Sherratt M J. Molecular aspects of skin ageing. Maturitas. 2011 July; 69(3): 249-56.

23. Ortiz-Urda S, Thyagarajan B, Keene D R, Lin Q, Fang M, Calos M P, Khavari P A. Stable nonviral genetic correction of inherited human skin disease. Nat Med. 2002 October; 8(10): 1166-70. Epub 2002 Sep. 16. Erratum in: Nat Med. 2003 February; 9(2): 237.

24. Osborn M J, Starker C G, McElroy A N, Webber B R, Riddle M J, Xia L, DeFeo A P, Gabriel R, Schmidt M, von Kalle C, Carlson D F, Maeder M L, Joung J K, Wagner J E, Voytas D F, Blazar B R, Tolar J. TALEN-based gene correction for epidermolysis bullosa. Mol Ther. 2013 June; 21(6): 1151-9.

25. Raveendran M, Senthil D, Utama B, Shen Y, Dudley D, Wang J, Zhang Y, Wang X L. Cigarette suppresses the expression of P4Halpha and vascular collagen production. Biochem Biophys Res Commun. 2004 Oct. 15; 323(2): 592-8.

26. Reflection paper on design modifications of gene therapy medicinal products during development/14 Dec. 2011 EMA/CAT/GTWP/44236/2009 Committee for advanced therapies 27. Ritz-Timme et al., 2003

28. Santos V J. High Throughput Screen for molecules and metabolites with potential cosmetic application UNIVERSIDADE DE LISBOA, Mestrado em Microbiologia Aplicada,2010.

29. Seltmann K, Meyer M, Sulcova J, Kockmann T, Wehkamp U, Weidinger S, Auf dem Keller U, Werner S. Humidity-regulated CLCA2 protects the epidermis from hyperosmotic stress. Sci Transl Med. 2018 May 9; 10(440).

30. Talwar H S, Griffiths C E, Fisher G J, Hamilton T A, Voorhees J J. Reduced type I and type III procollagens in photodamaged adult human skin. J Invest Dermatol.

31. 1995 August; 105(2): 285-90. Tasker P N, Macdonald H, Fraser W D, Reid D M, Ralston S H, Albagha O M. Association of PLOD1 polymorphisms with bone mineral density in a population-based study of women from the UK. Osteoporos Int. 2006; 17(7): 1078-85.

32. Uchinaka A, Kawaguchi N, Hamada Y, Miyagawa S, Saito A, Mori S, Sawa Y, Matsuura N. Transplantation of elastin-secreting myoblast sheets improves cardiac function in infarcted rat heart. Mol Cell Biochem. 2012 September; 368(1-2): 203-14.

33. van Dijk F S, Mancini G M S, Maugeri A, Cobben J M. Ehlers Danlos syndrome, kyphoscoliotic type due to Lysyl Hydroxylase 1 deficiency in two children without congenital or early onset kyphoscoliosis. Eur J Med Genet. 2017 October; 60(10): 536-540.

34. Woodley D T, Keene D R, Atha T, Huang Y, Ram R, Kasahara N, Chen M. Intradermal injection of lentiviral vectors corrects regenerated human dystrophic epidermolysis bullosa skin tissue in vivo. Mol Ther. 2004 Auguat; 10(2): 318-26.

35. Zou Y, Donkervoort S, Salo A M, Foley A R, Barnes A M, Hu Y, Makareeva E, Leach M E, Mohassel P, Dastgir J, Deardorff M A, Cohn R D, DiNonno W O, Malfait F, Lek M, Leikin S, Marini J C, Myllyharju J, Bonnemann C G. P4HA1 mutations cause a unique congenital disorder of connective tissue involving tendon, bone, muscle and the eye. Hum Mol Genet. 2017 Jun. 15; 26(12): 2207-2217.

36. Biochemistry: Textbook for higher education institutions, edited by E. S. Severin., 2003. p. 779 ISBN 5-9231-0254-4

37. Molecular Biology, 2011, Vol. 45, No. 1, p. 44-55

38. S. S. Kuznetsov, V. V. Dudenkova, M. V. Kochueva, E. B. Kiseleva, N. Yu. Ignatieva, O. L. Zakharkin, E. A. Sergeeva, K. V. Babak, A. V. Maslennikov. Multiphoton microscopy in the study of morphological characteristics of radiation-induced injuries of the bladder. Modern technologies in medicine, 2016, volume 8, number 2, p. 31-39.

39. Urology: textbook/B. K. Komyakov.—2012.—p. 464

What is claimed is:

1.-22. (canceled)

23. A gene therapy DNA vector based on a gene therapy DNA vector VTvaf17 for treatment of diseases associated with disorders of formation of an extracellular matrix of a skin and other organs, a skin structure, wound healing disorders, for prevention of a skin ageing caused by internal and external factors, for treatment of hereditary and acquired connective tissue diseases, including Ehlers-Danlos syndrome, epidermolysis bullosa, Caffey disease, osteogenesis imperfecta, pathological scarring, a formation of scars, superficial epidermal cysts, and a hyperpigmentation alopecia, wherein the gene therapy DNA vector has a coding region of a therapeutic gene, selected from COL1A1, or COL1A2, or P4HA1, or P4HA2, or COL7A1, or CLCA2, or ELN, or PLOD1, cloned to the gene therapy DNA vector VTvaf17 resulting in a gene therapy DNA vector VTvaf17-COL1A1 that has a nucleotide sequence SEQ ID No. 1, or resulting in a gene therapy DNA vector VTvaf17-COL1A2 that has a nucleotide sequence SEQ ID No. 2, or resulting in a gene therapy DNA vector VTvaf17-P4HA1 that has a nucleotide sequence SEQ ID No. 3, or resulting in a gene therapy DNA vector VTvaf17-P4HA2 that has a nucleotide sequence SEQ ID No. 4, or resulting in a gene therapy DNA vector VTvaf17-COL7A1 that has a nucleotide sequence SEQ ID No. 5, or resulting in a gene therapy DNA vector VTvaf17-CLCA2 that has a nucleotide sequence SEQ ID No. 6, or resulting in a gene therapy DNA vector VTvaf17-ELN that has a nucleotide sequence SEQ ID No. 7, or resulting in a gene therapy DNA vector VTvaf17-PLOD1 that has a nucleotide sequence SEQ ID No. 8 respectively.

24. The gene therapy DNA vector based on the gene therapy DNA vector VTvaf17 carrying COL1A1, or COL1A2, or P4HA1, or P4HA2, or COL7A1, or CLCA2, or ELN, or PLOD1 therapeutic gene as per claim 23, the gene therapy DNA vectors being unique due to a fact that each of the gene therapy DNA vectors: VTvaf17-COL1A1, or VTvaf17-COL1A2, or VTvaf17-P4HA1, or VTvaf17-P4HA2, or VTvaf17-COL7A1, or VTvaf17-CLCA2, or VTvaf17-ELN, or VTvaf17-PLOD1 as per claim 23 uses the nucleotide sequences that are not antibiotic resistance genes, virus genes, or regulatory elements of viral genomes as structure elements, which ensures a safe use for the gene therapy in humans and animals.

25. A method of a gene therapy DNA vector production based on the gene therapy DNA vector VTvaf17 carrying COL1A1, or COL1A2, or P4HA1, or P4HA2, or COL7A1, or CLCA2, or ELN, or PLOD1 therapeutic gene as per claim 23 that involves obtaining each of the gene therapy DNA vectors: VTvaf17-COL1A1, or VTvaf17-COL1A2, or VTvaf17-P4HA1, or VTvaf17-P4HA2, or VTvaf17-COL7A1, or VTvaf17-CLCA2, or VTvaf17-ELN, or VTvaf17-PLOD' as follows: the coding region of the COL1A1, or COL1A2, or P4HA1, or P4HA2, or COL7A1, or CLCA2, or ELN, or PLOD1 therapeutic gene as per claim 23 is cloned to the gene therapy DNA vector VTvaf17, and the gene therapy DNA vector VTvaf17-COL1A1, SEQ ID No. 1, or VTvaf17-COL1A2, SEQ ID No. 2 or VTvaf17-P4HA1, SEQ ID No. 3, or VTvaf17-P4HA2, SEQ ID No. 4, or VTvaf17-COL7A1, SEQ ID No. 5, or VTvaf17-CLCA2, SEQ ID No. 6, or VTvaf17-ELN, SEQ ID No. 7, or VTvaf17-PLOD1, SEQ ID No. 8, respectively, is obtained, while the coding region of the COL1A1, or COL1A2, or P4HA1, or P4HA2, or COL7A1, or CLCA2, or ELN, or PLOD1 therapeutic gene is obtained by isolating a total RNA from a human biological tissue sample followed by a reverse transcription reaction and a PCR amplification using the obtained oligonucleotides and cleaving an amplification product by corresponding restriction endonucleases, wherein cloning to the gene therapy DNA vector VTvaf17 is performed by Nhel and Hindlll, restriction sites, wherein a selection is performed without antibiotics, at the same time, the following oligonucleotides are used during the gene therapy DNA vector VTvaf17-COL1A1, SEQ ID No. 1 production for the reverse transcription reaction and the PCR amplification:
Col1A1_F
CCAGCTAGCGTCTAGGGTCTAGACATGTTC,
Col1A1_R
TATAAGCTTCTACAGGAAGCAGACAGGGCCAAC,
and the cleaving of the amplification product and cloning of the coding region of COL1A1 gene to gene therapy DNA vector VTvaf17 is performed by Nhel and Hindlll restriction endonucleases,
at the same time, the following oligonucleotides are used during the gene therapy DNA vector VTvaf17-COL1A2, SEQ ID No. 2 production for the reverse transcription reaction and the PCR amplification:
Col1A2_F
CCAGCTAGCGTCTAAGTGCTAGACATGCTC,
Col1A2_R
CGAAGCTTTTATTTGAAACAGACTGGGCCA,
and the cleaving of the amplification product and cloning of the coding region of COL1A2 gene to the gene therapy DNA vector VTvaf17 is performed by Nhel and Hindlll restriction endonucleases,
at the same time, the following oligonucleotides are used during the gene therapy DNA vector VTvaf17-P4HA1, SEQ ID No. 3 production for the reverse transcription reaction and the PCR amplification:
P4HA1_F
AGGATCCACCATGATCTGGTATATATTAATTATAGG,
P4HA1_R
TTCGGTACCTATTCCAATTCTGACAACGTACAAG,
and the cleaving of the amplification product and cloning of the coding region of P4HA1 gene to the gene therapy DNA vector VTvaf17 is performed by BamHI and KpnI restriction endonucleases,
at the same time, the following oligonucleotides are used during the gene therapy DNA vector VTvaf17-P4HA2, SEQ ID No. 4 production for the reverse transcription reaction and the PCR amplification:
P4HA2_F
AGGATCCACCATGAAACTCTGGGTGTCTGCA,
P4HA2_R
CTTGTCGACTTAGTCAACTTCTGTTGATCCACA,
and the cleaving of the amplification product and cloning of the coding region of P4HA2 gene to the gene therapy DNA vector VTvaf17 is performed by BamHII and SaiI restriction endonucleases,
at the same time, the following oligonucleotides are used during the gene therapy DNA vector VTvaf17-COL7A1, SEQ ID No. 5 production for the reverse transcription reaction and the PCR amplification:
COL7A1_F
ATCGTCGACCACCATGACGCTGCGGCTTCTGGT
COL7A1_R
ATAGAATTCAGTCCTGGGCAGTACCTGTC,
and the cleaving of the amplification product and cloning of the coding region of COL7A1 gene to the gene therapy DNA vector VTvaf17 is performed by SaiI and EcoRI restriction endonucleases,
at the same time, the following oligonucleotides are used during the gene therapy DNA vector VTvaf17-CLCA2, SEQ ID No. 6 production for the reverse transcription reaction and the PCR amplification:
CLCA2_F
AGGATCCACCATGACCCAAAGGAGCATTGC,
CLCA2_R
ATAGAATTCATAATAATTTTGTTCCATTCTCTTTC,
and the cleaving of the amplification product and cloning of the coding region of CLCA2 gene to the gene therapy DNA vector VTvaf17 is performed by BamHI and EcoRI restriction endonucleases,
at the same time, the following oligonucleotides are used during the gene therapy DNA vector VTvaf17-ELN, SEQ ID No. 7 production for the reverse transcription reaction and the PCR amplification:
ELN_F
TTTGTCGACCACCATGGCGGGTCTGACGGCGG,
ELN_R
TTTTTGAATTCTCATTTTCTCTTCCGGCCACAAGCTT,
and the cleaving of the amplification product and cloning of the coding region of ELN gene to the gene therapy DNA vector VTvaf17 is performed by SalI and EcoRI restriction endonucleases,
at the same time, the following oligonucleotides are used during the gene therapy DNA vector VTvaf17-PLOD1, SEQ ID No. 8 production for the reverse transcription reaction and the PCR amplification:
PLOD1_F
GGATCCACCATGCGGCCCCTGCTGCTACT,
PLOD1_R
ATAGAATTCAGGGATCGACGAAGGAGACT,
and the cleaving of the amplification product and cloning of the coding region of PLOD1 gene to the gene therapy DNA vector VTvaf17 is performed by BamHII and EcoRI restriction endonucleases.

26. A method of use of the gene therapy DNA vector based on the gene therapy DNA vector VTvaf17 carrying COL1A1, or COL1A2, or P4HA1, or P4HA2, or COL7A1, or CLCA2, or ELN, or PLOD1 therapeutic gene as per claim 23 for treatment of diseases associated with disorders of formation of the extracellular matrix of the skin and other organs, the skin structure, the wound healing disorders, for the prevention of the skin ageing caused by internal and external factors, for the treatment of hereditary and acquired connective tissue diseases, including Ehlers-Danlos syndrome, epidermolysis bullosa, Caffey disease, osteogenesis imperfecta, pathological scarring, formation of scars, superficial epidermal cysts, and hyperpigmentation that involves transfection of the cells of patient or animal organs and tissues with the selected gene therapy DNA vector carrying the therapeutic gene based on the gene therapy DNA vector VTvaf17, or several selected gene therapy DNA vectors carrying the therapeutic genes based on the gene therapy DNA vector VTvaf17, from a group of the constructed gene therapy DNA vectors carrying the therapeutic genes based on the gene therapy DNA vector VTvaf17 and an injection of autologous cells of said patient or animal transfected by the selected gene therapy DNA vector carrying the therapeutic gene based on the gene therapy DNA vector VTvaf17 or several selected gene therapy DNA vectors carrying the therapeutic genes based on the gene therapy DNA vector VTvaf17 from the constructed gene therapy DNA vectors carrying the therapeutic genes based on the gene therapy DNA vector VTvaf17 into the organs and tissues of the same patient or animal and the injection of the selected gene therapy DNA vector carrying the therapeutic gene based on the gene therapy DNA vector VTvaf17 or several selected gene therapy DNA vectors carrying the therapeutic genes based on the gene therapy DNA vector VTvaf17 from a group of constructed gene therapy DNA vectors carrying the therapeutic genes based on the gene therapy DNA vector VTvaf17 into the organs and tissues of the same patient or animal, or a combination of the indicated methods.


SEQUENCE LISTING (40 sequences)

SEQ ID NO: 1 - DNA, 7563 bp, Homo Sapiens
cgtgaggctccggtgcccgtcagtgggcagagcgcacatcgcccacagtccccgagaagttggggggaggggtcggcaattgaaccggtgcctagagaaagtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaactacccctaaaagccagctagcgtctagggtctagacatgttcagctttgtggacctccggctcctgctcctcttagcggccaccgccctcctgacgcacggccaagaggaaggccaagtcgagggccaagacgaagacatcccaccaatcacctgcgtacagaacggcctcaggtaccatgaccgagacgtgtggaaacccgagccctgccggatctgcgtctgcgacaacggcaaggtgttgtgcgatgacgtgatctgtgacgagaccaagaactgccccggcgccgaagtccccgagggcgagtgctgtcccgtctgccccgacggctcagagtcacccaccgaccaagaaaccaccggcgtcgagggacccaagggagacactggcccccgaggcccaaggggacccgcaggcccccctggccgagatggcatccctggacagcctggacttcccggaccccccggaccccccggacctcccggaccccctggcctcggaggaaactttgctccccagctgtcttatggctatgatgagaaatcaaccggaggaatttccgtgcctggccccatgggtccctctggtcctcgtggtctccctggcccccctggtgcacctggtccccaaggcttccaaggtccccctggtgagcctggcgagcctggagcttcaggtcccatgggtccccgaggtcccccaggtccccctggaaagaatggagatgatggggaagctggaaaacctggtcgtcctggtgagcgtgggcctcctgggcctcagggtgctcgaggattgcccggaacagctggcctccctggaatgaagggacacagaggtttcagtggtttggatggtgccaagggagatgctggtcctgctggtcctaagggtgagcctggcagccctggtgaaaatggagctcctggtcagatgggcccccgtggcctgcctggtgagagaggtcgccctggagcccctggccctgctggtgctcgtggaaatgatggtgctactggtgctgccgggccccctggtcccaccggccccgctggtcctcctggcttccctggtgctgttggtgctaagggtgaagctggtccccaagggccccgaggctctgaaggtccccagggtgtgcgtggtgagcctggcccccctggccctgctggtgctgctggccctgctggaaaccctggtgctgatggacagcctggtgctaaaggtgccaatggtgctcctggtattgctggtgctcctggcttccctggtgcccgaggcccctctggaccccagggccccggcggccctcctggtcccaagggtaacagcggtgaacctggtgctcctggcagcaaaggagacactggtgctaagggagagcctggccctgttggtgttcaaggaccccctggccctgctggagaggaaggaaagcgaggagctcgaggtgaacccggacccactggcctgcccggaccccctggcgagcgtggtggacctggtagccgtggtttccctggcgcagatggtgttgctggtcccaagggtcccgctggtgaacgtggttctcctggccccgctggccccaaaggatctcctggtgaagctggtcgtcccggtgaagctggtctgcctggtgccaagggtctgactggaagccctggcagccctggtcctgatggcaaaactggcccccctggtcccgccggtcaagatggtcgccccggacccccaggcccacctggtgcccgtggtcaggctggtgtgatgggattccctggacctaaaggtgctgctggagagcccggcaaggctggagagcgaggtgttcccggaccccctggcgctgtcggtcctgctggcaaagatggagaggctggagctcagggaccccctggccctgctggtcccgctggcgagagaggtgaacaaggccctgctggctcccccggattccagggtctccctggtcctgctggtcctccaggtgaagcaggcaaacctggtgaacagggtgttcctggagaccttggcgcccctggcccctctggagcaagaggcgagagaggtttccctggcgagcgtggtgtgcaaggtccccctggtcctgctggaccccgaggggccaacggtgctcccggcaacgatggtgctaagggtgatgctggtgcccctggagctcccggtagccagggcgcccctggccttcagggaatgcctggtgaacgtggtgcagctggtcttccagggcctaagggtgacagaggtgatgctggtcccaaaggtgctgatggctctcctggcaaagatggcgtccgtggtctgaccggccccattggtcctcctggccctgctggtgcccctggtgacaagggtgaaagtggtcccagcggccctgctggtcccactggagctcgtggtgcccccggagaccgtggtgagcctggtccccccggccctgctggctttgctggcccccctggtgctgacggccaacctggtgctaaaggcgaacctggtgatgctggtgccaaaggcgatgctggtccccctgggcctgccggacccgctggaccccctggccccattggtaatgttggtgctcctggagccaaaggtgctcgcggcagcgctggtccccctggtgctactggtttccctggtgctgctggccgagtcggtcctcctggcccctctggaaatgctggaccccctggccctcctggtcctgctggcaaagaaggcggcaaaggtccccgtggtgagactggccctgctggacgtcctggtgaagttggtccccctggtccccctggccctgctggcgagaaaggatcccctggtgctgatggtcctgctggtgctcctggtactcccgggcctcaaggtattgctggacagcgtggtgtggtcggcctgcctggtcagagaggagagagaggcttccctggtcttcctggcccctctggtgaacctggcaaacaaggtccctctggagcaagtggtgaacgtggtccccccggtcccatgggcccccctggattggctggaccccctggtgaatctggacgtgagggggctcctgctgccgaaggttcccctggacgagacggttctcctggcgccaagggtgaccgtggtgagaccggccccgctggaccccctggtgctcctggtgctcctggtgcccctggccccgttggccctgctggcaagagtggtgatcgtggtgagactggtcctgctggtcccgccggtcccgtcggccccgtcggcgcccgtggccccgccggaccccaaggcccccgtggtgacaagggtgagacaggcgaacagggcgacagaggcataaagggtcaccgtggcttctctggcctccagggtccccctggccctcctggctctcctggtgaacaaggtccctctggagcctctggtcctgctggtccccgaggtccccctggctctgctggtgctcctggcaaagatggactcaacggtctccctggccccattgggccccctggtcctcgcggtcgcactggtgatgctggtcctgttggtccccccggccctcctggacctcctggtccccctggtcctcccagcgctggtttcgacttcagcttcctgccccagccacctcaagagaaggctcacgatggtggccgctactaccgggctgatgatgccaatgtggttcgtgaccgtgacctcgaggtggacaccaccctcaagagcctgagccagcagatcgagaacatccggagcccagagggaagccgcaagaaccccgcccgcacctgccgtgacctcaagatgtgccactctgactggaagagtggagagtactggattgaccccaaccaaggctgcaacctggatgccatcaaagtcttctgcaacatggagactggtgagacctgcgtgtaccccactcagcccagtgtggcccagaagaactggtacatcagcaagaaccccaaggacaagaggcatgtctggttcggcgagagcatgaccgatggattccagttcgagtatggcggccagggctccgaccctgccgatgtggccatccagctgaccttcctgcgcctgatgtccaccgaggcctcccagaacatcacctaccactgcaagaacagcgtggcctacatggaccagcagactggcaacctcaagaaggccctgctcctcaagggctccaacgagatcgagatccgcgccgagggcaacagccgcttcacctacagcgtcactgtcgatggctgcacgagtcacaccggagcctggggcaagacagtgattgaatacaaaaccaccaagtcctcccgcctgcccatcatcgatgtggcccccttggacgttggtgccccagaccaggaattcggcttcgacgttggccctgtctgcttcctgtagaagcttggtaccgaattccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttacgcgtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaactagtccatggctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcc

SEQ ID NO: 2 - DNA, 7269 bp, Homo Sapiens
cgtgaggctccggtgcccgtcagtgggcagagcgcacatcgcccacagtccccgagaagttggggggaggggtcggcaattgaaccggtgcctagagaaagtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaactacccctaaaagccagctagcgtctaagtgctagacatgctcagctttgtggatacgcggactttgttgctgcttgcagtaaccttatgcctagcaacatgccaatctttacaagaggaaactgtaagaaagggcccagccggagatagaggaccacgtggagaaaggggtccaccaggccccccaggcagagatggtgaagatggtcccacaggccctcctggtccacctggtcctcctggcccccctggtctcggtgggaactttgctgctcagtatgatggaaaaggagttggacttggccctggaccaatgggcttaatgggacctagaggcccacctggtgcagctggagccccaggccctcaaggtttccaaggacctgctggtgagcctggtgaacctggtcaaactggtcctgcaggtgctcgtggtccagctggccctcctggcaaggctggtgaagatggtcaccctggaaaacccggacgacctggtgagagaggagttgttggaccacagggtgctcgtggtttccctggaactcctggacttcctggcttcaaaggcattaggggacacaatggtctggatggattgaagggacagcccggtgctcctggtgtgaagggtgaacctggtgcccctggtgaaaatggaactccaggtcaaacaggagcccgtgggcttcctggtgagagaggacgtgttggtgcccctggcccagctggtgcccgtggcagtgatggaagtgtgggtcccgtgggtcctgctggtcccattgggtctgctggccctccaggcttcccaggtgcccctggccccaagggtgaaattggagctgttggtaacgctggtcctgctggtcccgccggtccccgtggtgaagtgggtcttccaggcctctccggccccgttggacctcctggtaatcctggagcaaacggccttactggtgccaagggtgctgctggccttcccggcgttgctggggctcccggcctccctggaccccgcggtattcctggccctgttggtgctgccggtgctactggtgccagaggacttgttggtgagcctggtccagctggctccaaaggagagagcggtaacaagggtgagcccggctctgctgggccccaaggtcctcctggtcccagtggtgaagaaggaaagagaggccctaatggggaagctggatctgccggccctccaggacctcctgggctgagaggtagtcctggttctcgtggtcttcctggagctgatggcagagctggcgtcatgggccctcctggtagtcgtggtgcaagtggccctgctggagtccgaggacctaatggagatgctggtcgccctggggagcctggtctcatgggacccagaggtcttcctggttcccctggaaatatcggccccgctggaaaagaaggtcctgtcggcctccctggcatcgacggcaggcctggcccaattggccccgctggagcaagaggagagcctggcaacattggattccctggacccaaaggccccactggtgatcctggcaaaaacggtgataaaggtcatgctggtcttgctggtgctcggggtgctccaggtcctgatggaaacaatggtgctcagggacctcctggaccacagggtgttcaaggtggaaaaggtgaacagggtcccgctggtcctccaggcttccagggtctgcctggcccctcaggtcccgctggtgaagttggcaaaccaggagaaaggggtctccatggtgagtttggtctccctggtcctgctggtccaagaggggaacgcggtcccccaggtgagagtggtgctgccggtcctactggtcctattggaagccgaggtccttctggacccccagggcctgatggaaacaagggtgaacctggtgtggttggtgctgtgggcactgctggtccatctggtcctagtggactcccaggagagaggggtgctgctggcatacctggaggcaagggagaaaagggtgaacctggtctcagaggtgaaattggtaaccctggcagagatggtgctcgtggtgctcctggtgctgtaggtgcccctggtcctgctggagccacaggtgaccggggcgaagctggggctgctggtcctgctggtcctgctggtcctcggggaagccctggtgaacgtggtgaggtcggtcctgctggccccaatggatttgctggtcctgctggtgctgctggtcaacctggtgctaaaggagaaagaggagccaaagggcctaagggtgaaaacggtgttgttggtcccacaggccccgttggagctgctggcccagctggtccaaatggtccccccggtcctgctggaagtcgtggtgatggaggcccccctggtatgactggtttccctggtgctgctggacggactggtcccccaggaccctctggtatttctggccctcctggtccccctggtcctgctgggaaagaagggcttcgtggtcctcgtggtgaccaaggtccagttggccgaactggagaagtaggtgcagttggtccccctggcttcgctggtgagaagggtccctctggagaggctggtactgctggacctcctggcactccaggtcctcagggtcttcttggtgctcctggtattctgggtctccctggctcgagaggtgaacgtggtctaccaggtgttgctggtgctgtgggtgaacctggtcctcttggcattgccggccctcctggggcccgtggtcctcctggtgctgtgggtagtcctggagtcaacggtgctcctggtgaagctggtcgtgatggcaaccctgggaacgatggtcccccaggtcgcgatggtcaacccggacacaagggagagcgcggttaccctggcaatattggtcccgttggtgctgcaggtgcacctggtcctcatggccccgtgggtcctgctggcaaacatggaaaccgtggtgaaactggtccttctggtcctgttggtcctgctggtgctgttggcccaagaggtcctagtggcccacaaggcattcgtggcgataagggagagcccggtgaaaaggggcccagaggtcttcctggcttaaagggacacaatggattgcaaggtctgcctggtatcgctggtcaccatggtgatcaaggtgctcctggctccgtgggtcctgctggtcctaggggccctgctggtccttctggccctgctggaaaagatggtcgcactggacatcctggtacagttggacctgctggcattcgaggccctcagggtcaccaaggccctgctggcccccctggtccccctggccctcctggacctccaggtgtaagcggtggtggttatgactttggttacgatggagacttctacagggctgaccagcctcgctcagcaccttctctcagacccaaggactatgaagttgatgctactctgaagtctctcaacaaccagattgagacccttcttactcctgaaggctctagaaagaacccagctcgcacatgccgtgacttgagactcagccacccagagtggagcagtggttactactggattgaccctaaccaaggatgcactatggatgctatcaaagtatactgtgatttctctactggcgaaacctgtatccgggcccaacctgaaaacatcccagccaagaactggtataggagctccaaggacaagaaacacgtctggctaggagaaactatcaatgctggcagccagtttgaatataatgtagaaggagtgacttccaaggaaatggctacccaacttgccttcatgcgcctgctggccaactatgcctctcagaacatcacctaccactgcaagaacagcattgcatacatggatgaggagactggcaacctgaaaaaggctgtcattctacagggctctaatgatgttgaacttgttgctgagggcaacagcaggttcacttacactgttcttgtagatggctgctctaaaaagacaaatgaatggggaaagacaatcattgaatacaaaacaaataagccatcacgcctgcccttccttgatattgcacctttggacatcggtggtgctgaccaggaattctttgtggacattggcccagtctgtttcaaataaaagcttggtaccgaattccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttacgcgtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaactagtccatggctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcc

SEQ ID NO: 3 - DNA, 4754 bp, Homo Sapiens
cgtgaggctccggtgcccgtcagtgggcagagcgcacatcgcccacagtccccgagaagttggggggaggggtcggcaattgaaccggtgcctagagaaagtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaactacccctaaaagccaggatccaccatgatctggtatatattaattataggaattctgcttccccagtctttggctcatccaggcttttttacttcaattggtcagatgactgatttgatccatactgagaaagatctggtgacttctctgaaagattatattaaggcagaagaggacaagttagaacaaataaaaaaatgggcagagaagttagatcggctaactagtacagcgacaaaagatccagaaggatttgttgggcatccagtaaatgcattcaaattaatgaaacgtctgaatactgagtggagtgagttggagaatctggtccttaaggatatgtcagatggctttatctctaacctaaccattcagagacagtactttcctaatgatgaagatcaggttggggcagccaaagctctgttacgtctccaggatacctacaatttggatacagataccatctcaaagggtaatcttccaggagtgaaacacaaatcttttctaacggctgaggactgctttgagttgggcaaagtggcctatacagaagcagattattaccatacggaactgtggatggaacaagccctaaggcaactggatgaaggcgagatttctaccatagataaagtctctgttctagattatttgagctatgcggtatatcagcagggagacctggataaggcacttttgctcacaaagaagcttcttgaactagatcctgaacatcagagagctaatggtaacttaaaatattttgagtatataatggctaaagaaaaagatgtcaataagtctgcttcagatgaccaatctgatcagaaaactacaccaaagaaaaaaggggttgctgtggattacctgccagagagacagaagtacgaaatgctgtgccgtggggagggtatcaaaatgacccctcggagacagaaaaaactcttttgccgctaccatgatggaaaccgtaatcctaaatttattctggctccagctaaacaggaggatgaatgggacaagcctcgtattattcgcttccatgatattatttctgatgcagaaattgaaatcgtcaaagacctagcaaaaccaaggctgaggcgagccaccatttcaaacccaataacaggagacttggagacggtacattacagaattagcaaaagtgcctggctctctggctatgaaaatcctgtggtgtctcgaattaatatgagaatacaagatctaacaggactagatgtttccacagcagaggaattacaggtagcaaattatggagttggaggacagtatgaaccccattttgactttgcacggaaagatgagccagatgctttcaaagagctggggacaggaaatagaattgctacatggctgttttatatgagtgatgtgtctgcaggaggagccactgtttttcctgaagttggagctagtgtttggcccaaaaaaggaactgctgttttctggtataatctgtttgccagtggagaaggagattatagtacacggcatgcagcctgtccagtgctagttggcaacaaatgggtatccaataaatggctccatgaacgtggacaagaatttcgaagaccttgtacgttgtcagaattggaataggtaccgaattccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttacgcgtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaactagtccatggctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcc

SEQ ID NO: 4 - DNA, 4764 bp, Homo Sapiens
cgtgaggctccggtgcccgtcagtgggcagagcgcacatcgcccacagtccccgagaagttggggggaggggtcggcaattgaaccggtgcctagagaaagtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaactacccctaaaagccaggatccaccatgaaactctgggtgtctgcattgctgatggcctggtttggtgtcctgagctgtgtgcaggccgaattcttcacctctattgggcacatgactgacctgatttatgcagagaaagagctggtgcagtctctgaaagagtacatccttgtggaggaagccaagctttccaagattaagagctgggccaacaaaatggaagccttgactagcaagtcagctgctgatgctgagggctacctggctcaccctgtgaatgcctacaaactggtgaagcggctaaacacagactggcctgcgctggaggaccttgtcctgcaggactcagctgcaggttttatcgccaacctctctgtgcagcggcagttcttccccactgatgaggacgagataggagctgccaaagccctgatgagacttcaggacacatacaggctggacccaggcacaatttccagaggggaacttccaggaaccaagtaccaggcaatgctgagtgtggatgactgctttgggatgggccgctcggcctacaatgaaggggactattatcatacggtgttgtggatggagcaggtgctaaagcagcttgatgccggggaggaggccaccacaaccaagtcacaggtgctggactacctcagctatgctgtcttccagttgggtgatctgcaccgtgccctggagctcacccgccgcctgctctcccttgacccaagccacgaacgagctggagggaatctgcggtactttgagcagttattggaggaagagagagaaaaaacgttaacaaatcagacagaagctgagctagcaaccccagaaggcatctatgagaggcctgtggactacctgcctgagagggatgtttacgagagcctctgtcgtggggagggtgtcaaactgacaccccgtagacagaagaggcttttctgtaggtaccaccatggcaacagggccccacagctgctcattgcccccttcaaagaggaggacgagtgggacagcccgcacatcgtcaggtactacgatgtcatgtctgatgaggaaatcgagaggatcaaggagatcgcaaaacctaaacttgcacgagccaccgttcgtgatcccaagacaggagtcctcactgtcgccagctaccgggtttccaaaagctcctggctagaggaagatgatgaccctgttgtggcccgagtaaatcgtcggatgcagcatatcacagggttaacagtaaagactgcagaattgttacaggttgcaaattatggagtgggaggacagtatgaaccgcacttcgacttctctaggcgaccttttgacagcggcctcaaaacagaggggaataggttagcgacgtttcttaactacatgagtgatgtagaagctggtggtgccaccgtcttccctgatctgggggctgcaatttggcctaagaagggtacagctgtgttctggtacaacctcttgcggagcggggaaggtgactaccgaacaagacatgctgcctgccctgtgcttgtgggctgcaagtgggtctccaataagtggttccatgaacgaggacaggagttcttgagaccttgtggatcaacagaagttgactaagtcgacaagcttggtaccgaattccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttacgcgtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaactagtccatggctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcc

SEQ ID NO: 5 - DNA, 11990 bp, Homo Sapiens
cgtgaggctccggtgcccgtcagtgggcagagcgcacatcgcccacagtccccgagaagttggggggaggggtcggcaattgaaccggtgcctagagaaagtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaactacccctaaaagccaggatccgatatcgtcgaccaccatgacgctgcggcttctggtggccgcgctctgcgccgggatcctggcagaggcgccccgagtgcgagcccagcacagggagagagtgacctgcacgcgcctttacgccgctgacattgtgttcttactggatggctcctcatccattggccgcagcaatttccgcgaggtccgcagctttctcgaagggctggtgctgcctttctctggagcagccagtgcacagggtgtgcgctttgccacagtgcagtacagcgatgacccacggacagagttcggcctggatgcacttggctctgggggtgatgtgatccgcgccatccgtgagcttagctacaaggggggcaacactcgcacaggggctgcaattctccatgtggctgaccatgtcttcctgccccagctggcccgacctggtgtccccaaggtctgcatcctgatcacagacgggaagtcccaggacctggtggacacagctgcccaaaggctgaaggggcagggggtcaagctatttgctgtggggatcaagaatgctgaccctgaggagctgaagcgagttgcctcacagccaacctccgacttcttcttcttcgtcaatgacttcagcatcttgaggacactactgcccctcgtttcccggagagtgtgcacgactgctggtggcgtgcctgtgacccgacctccggatgactcgacctctgctccacgagacctggtgctgtctgagccaagcagccaatccttgagagtacagtggacagcggccagtggccctgtgactggctacaaggtccagtacactcctctgacggggctgggacagccactgccgagtgagcggcaggaggtgaacgtcccagctggtgagaccagtgtgcggctgcggggtctccggccactgaccgagtaccaagtgactgtgattgccctctacgccaacagcatcggggaggctgtgagcgggacagctcggaccactgccctagaagggccggaactgaccatccagaataccacagcccacagcctcctggtggcctggcggagtgtgccaggtgccactggctaccgtgtgacatggcgggtcctcagtggtgggcccacacagcagcaggagctgggccctgggcagggttcagtgttgctgcgtgacttggagcctggcacggactatgaggtgaccgtgagcaccctatttggccgcagtgtggggcccgccacttccctgatggctcgcactgacgcttctgttgagcagaccctgcgcccggtcatcctgggccccacatccatcctcctttcctggaacttggtgcctgaggcccgtggctaccggttggaatggcggcgtgagactggcttggagccaccgcagaaggtggtactgccctctgatgtgacccgctaccagttggatgggctgcagccgggcactgagtaccgcctcacactctacactctgctggagggccacgaggtggccacccctgcaaccgtggttcccactggaccagagctgcctgtgagccctgtaacagacctgcaagccaccgagctgcccgggcagcgggtgcgagtgtcctggagcccagtccctggtgccacccagtaccgcatcattgtgcgcagcacccagggggttgagcggaccctggtgcttcctgggagtcagacagcattcgacttggatgacgttcaggctgggcttagctacactgtgcgggtgtctgctcgagtgggtccccgtgagggcagtgccagtgtcctcactgtccgccgggagccggaaactccacttgctgttccagggctgcgggttgtggtgtcagatgcaacgcgagtgagggtggcctggggacccgtccctggagccagtggatttcggattagctggagcacaggcagtggtccggagtccagccagacactgcccccagactctactgccacagacatcacagggctgcagcctggaaccacctaccaggtggctgtgtcggtactgcgaggcagagaggagggccctgctgcagtcatcgtggctcgaacggacccactgggcccagtgaggacggtccatgtgactcaggccagcagctcatctgtcaccattacctggaccagggttcctggcgccacaggatacagggtttcctggcactcagcccacggcccagagaaatcccagttggtttctggggaggccacggtggctgagctggatggactggagccagatactgagtatacggtgcatgtgagggcccatgtggctggcgtggatgggccccctgcctctgtggttgtgaggactgcccctgagcctgtgggtcgtgtgtcgaggctgcagatcctcaatgcttccagcgacgttctacggatcacctgggtaggggtcactggagccacagcttacagactggcctggggccggagtgaaggcggccccatgaggcaccagatactcccaggaaacacagactctgcagagatccggggtctcgaaggtggagtcagctactcagtgcgagtgactgcacttgtcggggaccgcgagggcacacctgtctccattgttgtcactacgccgcctgaggctccgccagccctggggacgcttcacgtggtgcagcgcggggagcactcgctgaggctgcgctgggagccggtgcccagagcgcagggcttccttctgcactggcaacctgagggtggccaggaacagtcccgggtcctggggcccgagctcagcagctatcacctggacgggctggagccagcgacacagtaccgcgtgaggctgagtgtcctagggccggctggagaagggccctctgcagaggtgactgcgcgcactgagtcacctcgtgttccaagcattgaactacgtgtggtggacacctcgatcgactcggtgactttggcctggactccagtgtccagggcatccagctacatcctatcctggcggccactcagaggccctggccaggaagtgcctgggtccccgcagacacttccagggatctcaagctcccagcgggtgacagggctagagcctggcgtctcttacatcttctccctgacgcctgtcctggatggtgtgcggggtcctgaggcatctgtcacacagacgccagtgtgcccccgtggcctggcggatgtggtgttcctaccacatgccactcaagacaatgctcaccgtgcggaggctacgaggagggtcctggagcgtctggtgttggcacttgggcctcttgggccacaggcagttcaggttggcctgctgtcttacagtcatcggccctccccactgttcccactgaatggctcccatgaccttggcattatcttgcaaaggatccgtgacatgccctacatggacccaagtgggaacaacctgggcacagccgtggtcacagctcacagatacatgttggcaccagatgctcctgggcgccgccagcacgtaccaggggtgatggttctgctagtggatgaacccttgagaggtgacatattcagccccatccgtgaggcccaggcttctgggcttaatgtggtgatgttgggaatggctggagcggacccagagcagctgcgtcgcttggcgccgggtatggactctgtccagaccttcttcgccgtggatgatgggccaagcctggaccaggcagtcagtggtctggccacagccctgtgtcaggcatccttcactactcagccccggccagagccctgcccagtgtattgtccaaagggccagaagggggaacctggagagatgggcctgagaggacaagttgggcctcctggcgaccctggcctcccgggcaggaccggtgctcccggcccccaggggccccctggaagtgccactgccaagggcgagaggggcttccctggagcagatgggcgtccaggcagccctggccgcgccgggaatcctgggacccctggagcccctggcctaaagggctctccagggttgcctggccctcgtggggacccgggagagcgaggacctcgaggcccaaagggggagccgggggctcccggacaagtcatcggaggtgaaggacctgggcttcctgggcggaaaggggaccctggaccatcgggcccccctggacctcgtggaccactgggggacccaggaccccgtggccccccagggcttcctggaacagccatgaagggtgacaaaggcgatcgtggggagcggggtccccctggaccaggtgaaggtggcattgctcctggggagcctgggctgccgggtcttcccggaagccctggaccccaaggccccgttggcccccctggaaagaaaggagaaaaaggtgactctgaggatggagctccaggcctcccaggacaacctgggtctccgggtgagcagggcccacggggacctcctggagctattggccccaaaggtgaccggggctttccagggcccctgggtgaggctggagagaagggcgaacgtggacccccaggcccagcgggatcccgggggctgccaggggttgctggacgtcctggagccaagggtcctgaagggccaccaggacccactggccgccaaggagagaagggggagcctggtcgccctggggaccctgcagtggtgggacctgctgttgctggacccaaaggagaaaagggagatgtggggcccgctgggcccagaggagctaccggagtccaaggggaacggggcccacccggcttggttcttcctggagaccctggccccaagggagaccctggagaccggggtcccattggccttactggcagagcaggacccccaggtgactcagggcctcctggagagaagggagaccctgggcggcctggccccccaggacctgttggcccccgaggacgagatggtgaagttggagagaaaggtgacgagggtcctccgggtgacccgggtttgcctggaaaagcaggcgagcgtggccttcggggggcacctggagttcgggggcctgtgggtgaaaagggagaccagggagatcctggagaggatggacgaaatggcagccctggatcatctggacccaagggtgaccgtggggagccgggtcccccaggacccccgggacggctggtagacacaggacctggagccagagagaagggagagcctggggaccgcggacaagagggtcctcgagggcccaagggtgatcctggcctccctggagcccctggggaaaggggcattgaagggtttcggggacccccaggcccacagggggacccaggtgtccgaggcccagcaggagaaaagggtgaccggggtccccctgggctggatggccggagcggactggatgggaaaccaggagccgctgggccctctgggccgaatggtgctgcaggcaaagctggggacccagggagagacgggcttccaggcctccgtggagaacaaggcctccctggcccctctggtccccctggattaccgggaaagccaggcgaggatgggaaacctggcctgaatggaaaaaacggagaacctggggaccctggagaagacgggaggaagggagagaaaggagattcaggcgcctctgggagagaaggtcgtgatggccccaagggtgagcgtggagctcctggtatccttggaccccaggggcctccaggcctcccagggccagtgggccctcctggccagggttttcctggtgtcccaggaggcacgggccccaagggtgaccgtggggagactggatccaaaggggagcagggcctccctggagagcgtggcctgcgaggagagcctggaagtgtgccgaatgtggatcggttgctggaaactgctggcatcaaggcatctgccctgcgggagatcgtggagacctgggatgagagctctggtagcttcctgcctgtgcccgaacggcgtcgaggccccaagggggactcaggcgaacagggccccccaggcaaggagggccccatcggctttcctggagaacgcgggctgaagggcgaccgtggagaccctggccctcaggggccacctggtctggcccttggggagaggggcccccccgggccttccggccttgccggggagcctggaaagcctggtattcccgggctcccaggcagggctgggggtgtgggagaggcaggaaggccaggagagaggggagaacggggagagaaaggagaacgtggagaacagggcagagatggccctcctggactccctggaacccctgggccccccggaccccctggccccaaggtgtctgtggatgagccaggtcctggactctctggagaacagggaccccctggactcaagggtgctaagggggagccgggcagcaatggtgaccaaggtcccaaaggagacaggggtgtgccaggcatcaaaggagaccggggagagcctggaccgaggggtcaggacggcaacccgggtctaccaggagagcgtggtatggctgggcctgaagggaagccgggtctgcagggtccaagaggcccccctggcccagtgggtggtcatggagaccctggaccacctggtgccccgggtcttgctggccctgcaggaccccaaggaccttctggcctgaagggggagcctggagagacaggacctccaggacggggcctgactggacctactggagctgtgggacttcctggaccccccggcccttcaggccttgtgggtccacaggggtctccaggtttgcctggacaagtgggggagacagggaagccgggagccccaggtcgagatggtgccagtggaaaagatggagacagagggagccctggtgtgccagggtcaccaggtctgcctggccctgtcggacctaaaggagaacctggccccacgggggcccctggacaggctgtggtcgggctccctggagcaaagggagagaagggagcccctggaggccttgctggagacctggtgggtgagccgggagccaaaggtgaccgaggactgccagggccgcgaggcgagaagggtgaagctggccgtgcaggggagcccggagaccctggggaagatggtcagaaaggggctccaggacccaaaggtttcaagggtgacccaggagtcggggtcccgggctcccctgggcctcctggccctccaggtgtgaagggagatctgggcctccctggcctgcccggtgctcctggtgttgttgggttcccgggtcagacaggccctcgaggagagatgggtcagccaggccctagtggagagcggggtctggcaggccccccagggagagaaggaatcccaggacccctggggccacctggaccaccggggtcagtgggaccacctggggcctctggactcaaaggagacaagggagaccctggagtagggctgcctgggccccgaggcgagcgtggggagccaggcatccggggtgaagatggccgccccggccaggagggaccccgaggactcacggggccccctggcagcaggggagagcgtggggagaagggtgatgttgggagtgcaggactaaagggtgacaagggagactcagctgtgatcctggggcctccaggcccacggggtgccaagggggacatgggtgaacgagggcctcggggcttggatggtgacaaaggacctcggggagacaatggggaccctggtgacaagggcagcaagggagagcctggtgacaagggctcagccgggttgccaggactgcgtggactcctgggaccccagggtcaacctggtgcagcagggatccctggtgacccgggatccccaggaaaggatggagtgcctggtatccgaggagaaaaaggagatgttggcttcatgggtccccggggcctcaagggtgaacggggagtgaagggagcctgtggccttgatggagagaagggagacaagggagaagctggtcccccaggccgccccgggctggcaggacacaaaggagagatgggggagcctggtgtgccgggccagtcgggggcccctggcaaggagggcctgatcggtcccaagggtgaccgaggctttgacgggcagccaggccccaagggtgaccagggcgagaaaggggagcggggaaccccaggaattgggggcttcccaggccccagtggaaatgatggctctgctggtcccccagggccacctggcagtgttggtcccagaggccccgaaggacttcagggccagaagggtgagcgaggtccccccggagagagagtggtgggggctcctggggtccctggagctcctggcgagagaggggagcaggggcggccagggcctgccggtcctcgaggcgagaagggagaagctgcactgacggaggatgacatccggggctttgtgcgccaagagatgagtcagcactgtgcctgccagggccagttcatcgcatctggatcacgacccctccctagttatgctgcagacactgccggctcccagctccatgctgtgcctgtgctccgcgtctctcatgcagaggaggaagagcgggtaccccctgaggatgatgagtactctgaatactccgagtattctgtggaggagtaccaggaccctgaagctccttgggatagtgatgacccctgttccctgccactggatgagggctcctgcactgcctacaccctgcgctggtaccatcgggctgtgacaggcagcacagaggcctgtcacccttttgtctatggtggctgtggagggaatgccaaccgttttgggacccgtgaggcctgcgagcgccgctgcccaccccgggtggtccagagccaggggacaggtactgcccaggactgaattccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttacgcgtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaactagtccatggctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcc

SEQ ID NO: 6 - DNA, 5257 bp, Homo Sapiens
ggatccgatatcgtcgaccaccatggcgggactgacggcggcggccccgcggcccggagtcctcctgctcctgctgtccatcctccacccctctcggcctggaggggtccctggggccattcctggtggagttcctggaggagtcttttatccaggggctggtctcggagcccttggaggaggagcgctggggcctggaggcaaacctcttaagccagttcccggagggcttgcgggtgctggccttggggcagggctcggcgccttccccgcagttacctttccgggggctctggtgcctggtggagtggctgacgctgctgcagcctataaagctgctaaggctggcgctgggcttggtggtgtcccaggagttggtggcttaggagtgtctgcaggtgcggtggttcctcagcctggagccggagtgaagcctgggaaagtgccgggtgtggggctgccaggtgtatacccaggtggcgtgctcccaggagctcggttccccggtgtgggggtgctccctggagttcccactggagcaggagttaagcccaaggctccaggtgtaggtggagcttttgctggaatcccaggagttggaccctttgggggaccgcaacctggagtcccactggggtatcccatcaaggcccccaagctgcctggtggctatggactgccctacaccacagggaaactgccctatggctatgggcccggaggagtggctggtgcagcgggcaaggctggttacccaacagggacaggggttggcccccaggcagcagcagcagcggcagctaaagcagcagcaaagttcggtgctggagcagccggagtcctccctggtgttggaggggctggtgttcctggcgtgcctggggcaattcctggaattggaggcatcgcaggcgttgggactccagctgcagctgcagctgcagcagcagccgctaaggcagccaagtatggagctgctgcaggcttagtgcctggtgggccaggctttggcccgggagtagttggtgtcccaggagctggcgttccaggtgttggtgtcccaggagctgggattccagttgtcccaggtgctgggatcccaggtgctgcggttccaggggttgtgtcaccagaagcagctgctaaggcagctgcaaaggcagccaaatacggggccaggcccggagtcggagttggaggcattcctacttacggggttggagctgggggctttcccggctttggtgtcggagtcggaggtatccctggagtcgcaggtgtccctagtgtcggaggtgttcccggagtcggaggtgtcccgggagttggcatttcccccgaagctcaggcagcagctgccgccaaggctgccaagtacgggttagttcctggtgtcggcgtggctcctggagttggcgtggctcctggtgtcggtgtggctcctggagttggcttggctcctggagttggcgtggctcctggagttggtgtggctcctggcgttggcgtggctcccggcattggccctggtggagttgcagctgcagcaaaatccgctgccaaggtggctgccaaagcccagctccgagctgcagctgggcttggtgctggcatccctggacttggagttggtgtcggcgtccctggacttggagttggtgctggtgttcctggacttggagttggtgctggtgttcctggcttcggggcagtacctggagccctggttgccgctaaagcagccaaatatggagcagcagtgcctggggtccttggagggctcggggctctcggtggagtaggcatcccaggcggtgtggtgggagccagacccgccgccgccgctgccgcagccaaagctgctgccaaagccgcccagtttggcctagtgggagccgctgggctcggaggactcggagtcggagggcttggagttccaggtgttgggggccttggaggtatacctccagctgcagccgctaaagcagctaaatacggagtggcagcaagacctggcttcggattgtctcccattttcccaggtggggcctgcctggggaaagcttgtggccggaagagaaaatgagaattccgtgaggctccggtgcccgtcagtgggcagagcgcacatcgcccacagtccccgagaagttggggggaggggtcggcaattgaaccggtgcctagagaaagtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaactacccctaaaagccaggatccgatatcgtcgacaagcttggtaccgaattccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttacgcgtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaactagtccatggctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcc

SEQ ID NO: 7 - DNA, 5326 bp, Homo Sapiens
cgtgaggctccggtgcccgtcagtgggcagagcgcacatcgcccacagtccccgagaagttggggggaggggtcggcaattgaaccggtgcctagagaaagtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaactacccctaaaagccaggatccaccatgcggcccctgctgctactggccctgctgggctggctgctgctggccgaagcgaagggcgacgccaagccggaggacaaccttttagtcctcacggtggccactaaggagaccgagggattccgtcgcttcaagcgctcagctcagttcttcaactacaagatccaggcgcttggcctaggggaggactggaatgtggagaaggggacgtcggcaggtggagggcagaaggtccggctgctgaagaaagctctggagaagcacgcagacaaggaggatctggtcattctcttcgcagacagctatgacgtgctgtttgcatcggggccccgggagctcctgaagaagttccggcaggccaggagccaggtggtcttctctgctgaggagctcatctacccagaccgcaggctggagaccaagtatccggtggtgtccgatggcaagaggttcctgggctctggaggcttcatcggttatgcccccaacctcagcaaactggtggccgagtgggagggccaggacagcgacagcgatcagctgttttacaccaagatcttcttggacccggagaagagggagcagatcaatatcaccctggaccaccgctgccgtatcttccagaacctggatggagccttggatgaggtcgtgctcaagtttgaaatgggccatgtgagagcgaggaacctggcctatgacaccctcccggtcctgatccatggcaacgggccaaccaagctgcagttgaactacctgggcaactacatcccgcgcttctggaccttcgaaacaggctgcaccgtgtgtgacgaaggcttgcgcagcctcaagggcattggggatgaagctctgcccacggtcctggtcggcgtgttcatcgaacagcccacgccgtttgtgtccctgttcttccagcggctcctgcggctccactacccccagaaacacatgcgacttttcatccacaaccacgagcagcaccacaaggctcaggtggaagagttcctggcacagcatggcagcgagtaccagtctgtgaagctggtgggccctgaggtgcggatggcgaatgcagatgccaggaacatgggcgcagacctgtgccggcaggaccgcagctgcacctactacttcagcgtggatgctgacgtggccctgaccgagcccaacagcctgcggctgctgatccaacagaacaagaacgtcattgccccgctgatgacccggcatgggaggctgtggtcgaacttctggggggctctcagtgcagatggctactatgcccgttccgaggactacgtggacattgtgcaggggcggcgtgttggtgtctggaatgtgccctatatttcaaacatctacttgatcaagggcagtgccctgcggggtgagctgcagtcctcagatctcttccaccacagcaagctggaccccgacatggccttctgtgccaacatccggcagcaggatgtgttcatgttcctgaccaaccggcacacccttggccatctgctctccctagacagctaccgcaccacccacctgcacaacgacctctgggaggtgttcagcaaccccgaggactggaaggagaagtacatccaccagaactacaccaaagccctggcagggaagctggtggagacgccctgcccggatgtctattggttccccatcttcacggaggtggcctgtgatgagctggtggaggagatggagcactttggccagtggtctctgggcaacaacaaggacaaccgcatccagggtggctacgagaacgtgccgactattgacatccacatgaaccagatcggctttgagcgggagtggcacaaattcctgctggagtacattgcgcccatgacggagaagctctaccccggctactacaccagggcccagtttgacctggcctttgtcgtccgctacaagcctgatgagcagccctcactgatgccacaccatgatgcctccaccttcaccatcaacatcgccctgaaccgagtcggggtggattacgagggcgggggctgtcggttcctgcgctacaactgttccatccgagccccaaggaagggctggaccctcatgcaccctggacgactcacgcattaccatgaggggctccccaccaccaggggcacccgctacatcgcagtctccttcgtcgatccctgaattccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttacgcgtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaactagtccatggctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcc

SEQ ID NO: 8 - DNA, 5974 bp, Homo Sapiens
cgtgaggctccggtgcccgtcagtgggcagagcgcacatcgcccacagtccccgagaagttggggggaggggtcggcaattgaaccggtgcctagagaaagtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccgcacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaactacccctaaaagccaggatccaccatgacccaaaggagcattgcaggtcctatttgcaacctgaagtttgtgactctcctggttgccttaagttcagaactcccattcctgggagctggagtacagcttcaagacaatgggtataatggattgctcattgcaattaatcctcaggtacctgagaatcagaacctcatctcaaacattaaggaaatgataactgaagcttcattttacctatttaatgctaccaagagaagagtatttttcagaaatataaagattttaatacctgccacatggaaagctaataataacagcaaaataaaacaagaatcatatgaaaaggcaaatgtcatagtgactgactggtatggggcacatggagatgatccatacaccctacaatacagagggtgtggaaaagagggaaaatacattcatttcacacctaatttcctactgaatgataacttaacagctggctacggatcacgaggccgagtgtttgtccatgaatgggcccacctccgttggggtgtgttcgatgagtataacaatgacaaacctttctacataaatgggcaaaatcaaattaaagtgacaaggtgttcatctgacatcacaggcatttttgtgtgtgaaaaaggtccttgcccccaagaaaactgtattattagtaagctttttaaagaaggatgcacctttatctacaatagcacccaaaatgcaactgcatcaataatgttcatgcaaagtttatcttctgtggttgaattttgtaatgcaagtacccacaaccaagaagcaccaaacctacagaaccagatgtgcagcctcagaagtgcatgggatgtaatcacagactctgctgactttcaccacagctttcccatgaatgggactgagcttccacctcctcccacattctcgcttgtacaggctggtgacaaagtggtctgtttagtgctggatgtgtccagcaagatggcagaggctgacagactccttcaactacaacaagccgcagaattttatttgatgcagattgttgaaattcataccttcgtgggcattgccagtttcgacagcaaaggagagatcagagcccagctacaccaaattaacagcaatgatgatcgaaagttgctggtttcatatctgcccaccactgtatcagctaaaacagacatcagcatttgttcagggcttaagaaaggatttgaggtggttgaaaaactgaatggaaaagcttatggctctgtgatgatattagtgaccagcggagatgataagcttcttggcaattgcttacccactgtgctcagcagtggttcaacaattcactccattgccctgggttcatctgcagccccaaatctggaggaattatcacgtcttacaggaggtttaaagttctttgttccagatatatcaaactccaatagcatgattgatgctttcagtagaatttcctctggaactggagacattttccagcaacatattcagcttgaaagtacaggtgaaaatgtcaaacctcaccatcaattgaaaaacacagtgactgtggataatactgtgggcaacgacactatgtttctagttacgtggcaggccagtggtcctcctgagattatattatttgatcctgatggacgaaaatactacacaaataattttatcaccaatctaacttttcggacagctagtctttggattccaggaacagctaagcctgggcactggacttacaccctgaacaatacccatcattctctgcaagccctgaaagtgacagtgacctctcgcgcctccaactcagctgtgcccccagccactgtggaagcctttgtggaaagagacagcctccattttcctcatcctgtgatgatttatgccaatgtgaaacagggattttatcccattcttaatgccactgtcactgccacagttgagccagagactggagatcctgttacgctgagactccttgatgatggagcaggtgctgatgttataaaaaatgatggaatttactcgaggtattttttctcctttgctgcaaatggtagatatagcttgaaagtgcatgtcaatcactctcccagcataagcaccccagcccactctattccagggagtcatgctatgtatgtaccaggttacacagcaaacggtaatattcagatgaatgctccaaggaaatcagtaggcagaaatgaggaggagcgaaagtggggctttagccgagtcagctcaggaggctccttttcagtgctgggagttccagctggcccccaccctgatgtgtttccaccatgcaaaattattgacctggaagctgtaaaagtagaagaggaattgaccctatcttggacagcacctggagaagactttgatcagggccaggctacaagctatgaaataagaatgagtaaaagtctacagaatatccaagatgactttaacaatgctattttagtaaatacatcaaagcgaaatcctcagcaagctggcatcagggagatatttacgttctcaccccaaatttccacgaatggacctgaacatcagccaaatggagaaacacatgaaagccacagaatttatgttgcaatacgagcaatggataggaactccttacagtctgctgtatctaacattgcccaggcgcctctgtttattccccccaattctgatcctgtacctgccagagattatcttatattgaaaggagttttaacagcaatgggtttgataggaatcatttgccttattatagttgtgacacatcatactttaagcaggaaaaagagagcagacaagaaagagaatggaacaaaattattatgaattccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttacgcgtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaactagtccatggctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcc

SEQ ID NO: 9 - DNA, 30 bp, Artificial Sequence
  Oligonucleotide Col1A1_F
ccagctagcgtctagggtctagacatgttc

SEQ ID NO: 10 - DNA, 33 bp, Artificial Sequence
  Oligonucleotide Col1A1_R
tataagcttctacaggaagcagacagggccaac

SEQ ID NO: 11 - DNA, 20 bp, Artificial Sequence
  Oligonucleotide Col1A1_SF
tgacctcaagatgtgccact

SEQ ID NO: 12 - DNA, 21 bp, Artificial Sequence
  Oligonucleotide Col1A1_SR
cagacatgcctcttgtccttg

SEQ ID NO: 13 - DNA, 30 bp, Artificial Sequence
  Oligonucleotide Col1A2_F
ccagctagcgtctaagtgctagacatgctc

SEQ ID NO: 14 - DNA, 30 bp, Artificial Sequence
  Oligonucleotide Col1A2_R
cgaagcttttatttgaaacagactgggcca

SEQ ID NO: 15 - DNA, 20 bp, Artificial Sequence
  Oligonucleotide Col1A2_SF
tgaacttgttgctgagggca

SEQ ID NO: 16 - DNA, 20 bp, Artificial Sequence
  Oligonucleotide Col1A2_SR
ccagttcttggctgggatgt

SEQ ID NO: 17 - DNA, 36 bp, Artificial Sequence
  Oligonucleotide P4HA1_F
aggatccaccatgatctggtatatattaattatagg

SEQ ID NO: 18 - DNA, 34 bp, Artificial Sequence
  Oligonucleotide P4HA1_R
ttcggtacctattccaattctgacaacgtacaag

SEQ ID NO: 19 - DNA, 20 bp, Artificial Sequence
  Oligonucleotide P4HA1_SF
aaaagtgcctggctctctgg

SEQ ID NO: 20 - DNA, 20 bp, Artificial Sequence
  Oligonucleotide P4HA1_SR
tggctcatctttccgtgcaa

SEQ ID NO: 21 - DNA, 31 bp, Artificial Sequence
  Oligonucleotide P4HA2_F
aggatccaccatgaaactctgggtgtctgca

SEQ ID NO: 22 - DNA, 33 bp, Artificial Sequence
  Oligonucleotide P4HA2_R
cttgtcgacttagtcaacttctgttgatccaca

SEQ ID NO: 23 - DNA, 20 bp, Artificial Sequence
  Oligonucleotide P4HA2_SF
aggtaccaccatggcaacag

SEQ ID NO: 24 - DNA, 20 bp, Artificial Sequence
  Oligonucleotide P4HA2_SR
gtcttgggatcacgaacggt

SEQ ID NO: 25 - DNA, 33 bp, Artificial Sequence
  Oligonucleotide COL7A1_F
atcgtcgaccaccatgacgctgcggcttctggt

SEQ ID NO: 26 - DNA, 29 bp, Artificial Sequence
  Oligonucleotide COL7A1_R
atagaattcagtcctgggcagtacctgtc

SEQ ID NO: 27 - DNA, 20 bp, Artificial Sequence
  Oligonucleotide COL7A1_SF
caaaggagagatgggggagc

SEQ ID NO: 28 - DNA, 20 bp, Artificial Sequence
  Oligonucleotide COL7A1_SR
atcatttccactggggcctg

SEQ ID NO: 29 - DNA, 32 bp, Artificial Sequence
  Oligonucleotide ELN_F
tttgtcgaccaccatggcgggtctgacggcgg

SEQ ID NO: 30 - DNA, 37 bp, Artificial Sequence
  Oligonucleotide ELN_R
tttttgaattctcattttctcttccggccacaagctt

SEQ ID NO: 31 - DNA, 20 bp, Artificial Sequence
  Oligonucleotide ELN_SF
ccagtttggcctagtgggag

SEQ ID NO: 32 - DNA, 20 bp, Artificial Sequence
  Oligonucleotide ELN_SR
atgggagacaatccgaagcc

SEQ ID NO: 33 - DNA, 29 bp, Artificial Sequence
  Oligonucleotide PLOD1_F
ggatccaccatgcggcccctgctgctact

SEQ ID NO: 34 - DNA, 29 bp, Artificial Sequence
  Oligonucleotide PLOD1_R
atagaattcagggatcgacgaaggagact

SEQ ID NO: 35 - DNA, 20 bp, Artificial Sequence
  Oligonucleotide PLOD1_SF
gcctccaccttcaccatcaa

SEQ ID NO: 36 - DNA, 20 bp, Artificial Sequence
  Oligonucleotide PLOD1_SR
aaggagactgcgatgtagcg

SEQ ID NO: 37 - DNA, 30 bp, Artificial Sequence
  Oligonucleotide CLCA2_F
aggatccaccatgacccaaaggagcattgc

SEQ ID NO: 38 - DNA, 35 bp, Artificial Sequence
  Oligonucleotide CLCA2_R
atagaattcataataattttgttccattctctttc

SEQ ID NO: 39 - DNA, 20 bp, Artificial Sequence
  Oligonucleotide CLCA2_SF
ggaggctccttttcagtgct

SEQ ID NO: 40 - DNA, 20 bp, Artificial Sequence
  Oligonucleotide CLCA2_SR
gtagcctggccctgatcaaa